SEQUENCE LISTING (100 sequences)

SEQ ID NO: 1 - DNA, 23 bp, Artificial sequence
  Synthetic Construct
agggctatggggaggttgaagat

SEQ ID NO: 2 - DNA, 22 bp, Artificial sequence
  Synthetic Construct
actccagcaagttctgcagtca

SEQ ID NO: 3 - DNA, 24 bp, Artificial sequence
  Synthetic Construct
agggaataccagctcaacgactca

SEQ ID NO: 4 - DNA, 24 bp, Artificial sequence
  Synthetic Construct
tgtgtggggatgtagtcactctgt

SEQ ID NO: 5 - DNA, 22 bp, Artificial sequence
  Synthetic Construct
gagagtgaagaccacaggcatt

SEQ ID NO: 6 - DNA, 22 bp, Artificial sequence
  Synthetic Construct
cgttctgatctttggccaccta

SEQ ID NO: 7 - DNA, 22 bp, Artificial sequence
  Synthetic Construct
gagaccaagttccaggtggaca

SEQ ID NO: 8 - DNA, 20 bp, Artificial sequence
  Synthetic Construct
gatccacttgcggcgttcat

SEQ ID NO: 9 - DNA, 20 bp, Artificial sequence
  Synthetic Construct
atcagaacatcttcacggcc

SEQ ID NO: 10 - DNA, 20 bp, Artificial sequence
  Synthetic Construct
aaagcagacaccttctccac

SEQ ID NO: 11 - DNA, 22 bp, Artificial sequence
  Synthetic Construct
cggaatggatcacactgagaag

SEQ ID NO: 12 - DNA, 21 bp, Artificial sequence
  Synthetic Construct
acataaggatctgaaagcccg

SEQ ID NO: 13 - DNA, 20 bp, Artificial sequence
  Synthetic Construct
ttcccgatcccaaaagtgag

SEQ ID NO: 14 - DNA, 23 bp, Artificial sequence
  Synthetic Construct
gtcaaatcccaatcccaaatctc

SEQ ID NO: 15 - DNA, 23 bp, Artificial sequence
  Synthetic Construct
gcttcaaggttcacaactacatg

SEQ ID NO: 16 - DNA, 20 bp, Artificial sequence
  Synthetic Construct
accttctcccggcatttatg

SEQ ID NO: 17 - DNA, 20 bp, Artificial sequence
  Synthetic Construct
cctaccttctgcgatcactg

SEQ ID NO: 18 - DNA, 20 bp, Artificial sequence
  Synthetic Construct
tactttggcgattcctctgg

SEQ ID NO: 19 - DNA, 20 bp, Artificial sequence
  Synthetic Construct
gtaaatgcggtggaacttgc

SEQ ID NO: 20 - DNA, 20 bp, Artificial sequence
  Synthetic Construct
accccaatcccatttccttc

SEQ ID NO: 21 - DNA, 21 bp, Artificial sequence
  Synthetic Construct
gagaagcatgtgtttgagcag

SEQ ID NO: 22 - DNA, 21 bp, Artificial sequence
  Synthetic Construct
ggaagttttctttgtcgctgc

SEQ ID NO: 23 - DNA, 20 bp, Artificial sequence
  Synthetic Construct
acgaagtcaagagccacaag

SEQ ID NO: 24 - DNA, 20 bp, Artificial sequence
  Synthetic Construct
gtcgatgaaccacaaagctg

SEQ ID NO: 25 - DNA, 21 bp, Artificial sequence
  Synthetic Construct
cggattttggaatgtgcaagg

SEQ ID NO: 26 - DNA, 21 bp, Artificial sequence
  Synthetic Construct
cataaaggagaaccccgaagg

SEQ ID NO: 27 - DNA, 21 bp, Artificial sequence
  Synthetic Construct
tgcttacatttcctcatcccg

SEQ ID NO: 28 - DNA, 20 bp, Artificial sequence
  Synthetic Construct
cgcccgatgactctgattag

SEQ ID NO: 29 - DNA, 20 bp, Artificial sequence
  Synthetic Construct
aatggagggcaaaggagatg

SEQ ID NO: 30 - DNA, 21 bp, Artificial sequence
  Synthetic Construct
aagatgtaggcaatcactccg

SEQ ID NO: 31 - DNA, 21 bp, Artificial sequence
  Synthetic Construct
cttgagaaggatccgaacgag

SEQ ID NO: 32 - DNA, 20 bp, Artificial sequence
  Synthetic Construct
ttgcctccacttgctcttag

SEQ ID NO: 33 - DNA, 22 bp, Artificial sequence
  Synthetic Construct
cagttccagcgttcagttaatg

SEQ ID NO: 34 - DNA, 23 bp, Artificial sequence
  Synthetic Construct
ttcgtgtaggactcaaaatctcc

SEQ ID NO: 35 - DNA, 22 bp, Artificial sequence
  Synthetic Construct
ctctgacatcctgaactctgtg

SEQ ID NO: 36 - DNA, 22 bp, Artificial sequence
  Synthetic Construct
ccgtggtcttaatgatctcctg

SEQ ID NO: 37 - DNA, 22 bp, Artificial sequence
  Synthetic Construct
ggcacacctggatatctttctc

SEQ ID NO: 38 - DNA, 21 bp, Artificial sequence
  Synthetic Construct
agtctgtgttggtcttcatcc

SEQ ID NO: 39 - DNA, 20 bp, Artificial sequence
  Synthetic Construct
aaacagtctcgtaagcccag

SEQ ID NO: 40 - DNA, 20 bp, Artificial sequence
  Synthetic Construct
atcccatctgtagcgttgtg

SEQ ID NO: 41 - DNA, 19 bp, Artificial sequence
  Synthetic Construct
tcgcctctcacatccaaac

SEQ ID NO: 42 - DNA, 23 bp, Artificial sequence
  Synthetic Construct
catctcgctcactgtaatatccc

SEQ ID NO: 43 - DNA, 21 bp, Artificial sequence
  Synthetic Construct
aggacaccaacaacttcttcg

SEQ ID NO: 44 - DNA, 20 bp, Artificial sequence
  Synthetic Construct
ggtgccttgtcggtcatatt

SEQ ID NO: 45 - DNA, 22 bp, Artificial sequence
  Synthetic Construct
ctcagttcctggagaaagatgg

SEQ ID NO: 46 - DNA, 21 bp, Artificial sequence
  Synthetic Construct
cccagtcaattcatgtttgcc

SEQ ID NO: 47 - DNA, 22 bp, Artificial sequence
  Synthetic Construct
atggaatatgtgtctggaggtg

SEQ ID NO: 48 - DNA, 21 bp, Artificial sequence
  Synthetic Construct
tggtttcaggtctcgatgaac

SEQ ID NO: 49 - DNA, 21 bp, Artificial sequence
  Synthetic Construct
gagtaaacttcccctcaccag

SEQ ID NO: 50 - DNA, 20 bp, Artificial sequence
  Synthetic Construct
tccactgaccatccacaaag

SEQ ID NO: 51 - DNA, 20 bp, Artificial sequence
  Synthetic Construct
cactgttatccgctggtctg

SEQ ID NO: 52 - DNA, 21 bp, Artificial sequence
  Synthetic Construct
cttgtattggtgctctccctc

SEQ ID NO: 53 - DNA, 26 bp, Artificial sequence
  Synthetic Construct
caaggacaactcaaacttatacatgg

SEQ ID NO: 54 - DNA, 22 bp, Artificial sequence
  Synthetic Construct
cagatactcaaaggtcaggacg

SEQ ID NO: 55 - DNA, 21 bp, Artificial sequence
  Synthetic Construct
cctttccttgttcgactggag

SEQ ID NO: 56 - DNA, 19 bp, Artificial sequence
  Synthetic Construct
tgagctgcatagaaccgtg

SEQ ID NO: 57 - DNA, 22 bp, Artificial sequence
  Synthetic Construct
ccggatctccatcaatgagaag

SEQ ID NO: 58 - DNA, 20 bp, Artificial sequence
  Synthetic Construct
ttcaatccaaccctcccatc

SEQ ID NO: 59 - DNA, 20 bp, Artificial sequence
  Synthetic Construct
gcgcattctgaagttcctca

SEQ ID NO: 60 - DNA, 21 bp, Artificial sequence
  Synthetic Construct
aaaatccccagagccacatag

SEQ ID NO: 61 - DNA, 21 bp, Artificial sequence
  Synthetic Construct
ttgcccgttattgaccctatc

SEQ ID NO: 62 - DNA, 22 bp, Artificial sequence
  Synthetic Construct
cgttcctattccaagctcatcc

SEQ ID NO: 63 - DNA, 21 bp, Artificial sequence
  Synthetic Construct
tgactgcactggacatctttg

SEQ ID NO: 64 - DNA, 19 bp, Artificial sequence
  Synthetic Construct
tggttgtaggtttgctggg

SEQ ID NO: 65 - DNA, 23 bp, Artificial sequence
  Synthetic Construct
tgtcggagggaaatataaactgg

SEQ ID NO: 66 - DNA, 22 bp, Artificial sequence
  Synthetic Construct
ggccttctgagattctagcttc

SEQ ID NO: 67 - DNA, 18 bp, Artificial sequence
  Synthetic Construct
aggtggaggtagtggagg

SEQ ID NO: 68 - DNA, 22 bp, Artificial sequence
  Synthetic Construct
gtacaattgagtcagagtcccc

SEQ ID NO: 69 - DNA, 21 bp, Artificial sequence
  Synthetic Construct
gtgatgttctagccacagagg

SEQ ID NO: 70 - DNA, 21 bp, Artificial sequence
  Synthetic Construct
ccctttccctcctttcttgtc

SEQ ID NO: 71 - DNA, 21 bp, Artificial sequence
  Synthetic Construct
gttcaaatgcacccatcacag

SEQ ID NO: 72 - DNA, 21 bp, Artificial sequence
  Synthetic Construct
agtaactccccaggatctgtc

SEQ ID NO: 73 - DNA, 22 bp, Artificial sequence
  Synthetic Construct
gtatgagattctgaaggccctg

SEQ ID NO: 74 - DNA, 22 bp, Artificial sequence
  Synthetic Construct
ccaaaccccagtctattagtcg

SEQ ID NO: 75 - DNA, 22 bp, Artificial sequence
  Synthetic Construct
cgatacgaccatcaacagagac

SEQ ID NO: 76 - DNA, 20 bp, Artificial sequence
  Synthetic Construct
tcgctttccagtcttcatcg

SEQ ID NO: 77 - DNA, 1065 bp, Homo sapiens
atgggctgcacgctgagcgccgaggacaaggcggcggtggagcggagtaagatgatcgaccgcaacctccgtgaggacggcgagaaggcggcgcgcgaggtcaagctgctgctgctcggtgctggtgaatctggtaaaagtacaattgtgaagcagatgaaaattatccatgaagctggttattcagaagaggagtgtaaacaatacaaagcagtggtctacagtaacaccatccagtcaattattgctatcattagggctatggggaggttgaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtgacggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctctttgaagaaaaaatcaaaaagagccctctcactatatgctatccagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaa

SEQ ID NO: 78 - protein, 354 aa, Homo sapiens
MGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 79 - DNA, 909 bp, Homo sapiens
atgaaaattatccatgaagctggttattcagaagaggagtgtaaacaatacaaagcagtggtctacagtaacaccatccagtcaattattgctatcattagggctatggggaggttgaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtgacggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctctttgaagaaaaaatcaaaaagagccctctcactatatgctatccagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaa

SEQ ID NO: 80 - protein, 302 aa, Homo sapiens
MKIIHEAGYSEEECKQYKAVVYSNTIQSIIAIIRAMGRLKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 81 - DNA, 1068 bp, Homo sapiens
atgggctgcaccgtgagcgccgaggacaaggcggcggccgagcgctctaagatgatcgacaagaacctgcgggaggacggagagaaggcggcgcgggaggtgaagttgctgctgttgggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 82 - protein, 355 aa, Homo sapiens
MGCTVSAEDKAAAERSKMIDKNLREDGEKAAREVKLLLLGAGESGKSTIVKQMKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 83 - DNA, 957 bp, Homo sapiens
atgagaggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 84 - protein, 318 aa, Homo sapiens
MRGAGESGKSTIVKQMKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 85 - DNA, 912 bp, Homo sapiens
atgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 86 - protein, 303 aa, Homo sapiens
MKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 87 - DNA, 825 bp, Homo sapiens
atggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 88 - protein, 274 aa, Homo sapiens
MAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 89 - DNA, 1020 bp, Homo sapiens
atggagtatgcagggcatcttcctgccagctctgcccagggcaccatattggcatgcaccagctgcacaggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 90 - protein, 339 aa, Homo sapiens
MEYAGHLPASSAQGTILACTSCTGAGESGKSTIVKQMKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 91 - DNA, 1020 bp, Homo sapiens
atgacggagggtgtgaagacgctaggctggacgaagcagaaaggcgggtgtcactggggacgttctgagggtgctggggagtcagggaagagcaccatcgtcaagcagatgaagatcatccacgaggatggctactccgaggaggaatgccggcagtaccgggcggttgtctacagcaacaccatccagtccatcatggccattgtcaaagccatgggcaacctgcagatcgactttgccgacccctccagagcggacgacgccaggcagctatttgcactgtcctgcaccgccgaggagcaaggcgtgctccctgatgacctgtccggcgtcatccggaggctctgggctgaccatggtgtgcaggcctgctttggccgctcaagggaataccagctcaacgactcagctgcctactacctgaacgacctggagcgtattgcacagagtgactacatccccacacagcaagatgtgctacggacccgcgtaaagaccacggggatcgtggagacacacttcaccttcaaggacctacacttcaagatgtttgatgtgggtggtcagcggtctgagcggaagaagtggatccactgctttgagggcgtcacagccatcatcttctgcgtagccttgagcgcctatgacttggtgctagctgaggacgaggagatgaaccgcatgcatgagagcatgaagctattcgatagcatctgcaacaacaagtggttcacagacacgtccatcatcctcttcctcaacaagaaggacctgtttgaggagaagatcacacacagtcccctgaccatctgcttccctgagtacacaggggccaacaaatatgatgaggcagccagctacatccagagtaagtttgaggacctgaataagcgcaaagacaccaaggagatctacacgcacttcacgtgcgccaccgacaccaagaacgtgcagttcgtgtttgacgccgtcaccgatgtcatcatcaagaacaacctgaaggactgcggcctcttctga

SEQ ID NO: 92 - protein, 339 aa, Homo sapiens
MTEGVKTLGWTKQKGGCHWGRSEGAGESGKSTIVKQMKIIHEDGYSEEECRQYRAVVYSNTIQSIMAIVKAMGNLQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 93 - DNA, 1065 bp, Homo sapiens
atgggctgcacgttgagcgccgaagacaaggcggcagtggagcgaagcaagatgatcgaccgcaacttacgggaggacggggaaaaagcggccaaagaagtgaagctgctgctactcggtgctggagaatctggtaaaagcaccattgtgaaacagatgaaaatcattcatgaggatggctattcagaggatgaatgtaaacaatataaagtagttgtctacagcaatactatacagtccatcattgcaatcataagagccatgggacggctaaagattgactttggggaagctgccagggcagatgatgcccggcaattatttgttttagctggcagtgctgaagaaggagtcatgactccagaactagcaggagtgattaaacggttatggcgagatggtggggtacaagcttgcttcagcagatccagggaatatcagctcaatgattctgcttcatattatctaaatgatctggatagaatatcccagtctaactacattccaactcagcaagatgttcttcggacgagagtgaagaccacaggcattgtagaaacacatttcaccttcaaagacctatacttcaagatgtttgatgtaggtggccaaagatcagaacgaaaaaagtggattcactgttttgagggagtgacagcaattatcttctgtgtggccctcagtgattatgaccttgttctggctgaggacgaggagatgaaccgaatgcatgaaagcatgaaactgtttgacagcatttgtaataacaaatggtttacagaaacttcaatcattctcttccttaacaagaaagacctttttgaggaaaaaataaagaggagtccgttaactatctgttatccagaatacacaggttccaatacatatgaagaggcagctgcctatattcaatgccagtttgaagatctgaacagaagaaaagataccaaggagatctatactcacttcacctgtgccacagacacgaagaatgtgcagtttgtttttgatgctgttacagatgtcatcattaaaaacaacttaaaggaatgtggactttattga

SEQ ID NO: 94 - protein, 354 aa, Homo sapiens
MGCTLSAEDKAAVERSKMIDRNLREDGEKAAKEVKLLLLGAGESGKSTIVKQMKIIHEDGYSEDECKQYKVVVYSNTIQSIIAIIRAMGRLKIDFGEAARADDARQLFVLAGSAEEGVMTPELAGVIKRLWRDGGVQACFSRSREYQLNDSASYYLNDLDRISQSNYIPTQQDVLRTRVKTTGIVETHFTFKDLYFKMFDVGGQRSERKKWIHCFEGVTAIIFCVALSDYDLVLAEDEEMNRMHESMKLFDSICNNKWFTETSIILFLNKKDLFEEKIKRSPLTICYPEYTGSNTYEEAAAYIQCQFEDLNRRKDTKEIYTHFTCATDTKNVQFVFDAVTDVIIKNNLKECGLY

SEQ ID NO: 95 - DNA, 1185 bp, Homo sapiens
atgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 96 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 97 - DNA, 18 bp, Artificial sequence
  Synthetic Construct
gagagggaaagaagggga

SEQ ID NO: 98 - DNA, 23 bp, Artificial sequence
  Synthetic Construct
gaggaccgaaaagcgcaaagaca

SEQ ID NO: 99 - DNA, 15 bp, Artificial sequence
  Synthetic Construct
cagccaagacgccaa

SEQ ID NO: 100 - DNA, 22 bp, Artificial sequence
  Synthetic Construct
acaacaggcaccgggaggacaa

(19) United States
(12) Patent Application Publication
(10) Pub. No.: US 2018/0284132 A1
(43) Pub. Date: Oct. 4, 2018

(54) KITS FOR STRATIFYING SCOLIOTIC SUBJECTS

(71) Applicant: CHU SAINTE-JUSTINE, Montreal (CA)

(72) Inventors: Alain MOREAU, Montreal (CA); Marie-Yvonne AKOUME NDONG, Montreal (CA)

(21) Appl. No.: 15/960,765

(22) Filed: Apr. 24, 2018

Related U.S. Application Data
(62) Division of application No. 14/898,766, filed on Dec. 16, 2015, now abandoned.
(60) Provisional application No. 61/835,839, filed on Jun. 17, 2013.

(51) Int. Cl. G01N 33/68; A61K 31/4745; A61K 31/4188; A61K 31/18; A61K 31/4427

(52) U.S. Cl. CPC G01N 33/6872; G01N 2800/50; G01N 2440/14; G01N 2800/38; A61K 31/4745; A61K 31/4427; A61K 31/4188; G01N 2800/10; G01N 33/6893; A61K 31/18; G01N 2800/52; G01N 2333/4704

(57) ABSTRACT

Kits for stratifying a subject having or at risk for developing adolescent idiopathic scoliosis (AIS) into diagnostically or clinically useful subclasses are provided. The stratification is based on the subject's Giα protein serine phosphorylation profile and/or the degree of imbalance in G-protein coupled receptor responses to Giα and Gsα protein stimulation. In some embodiments, the methods involve detecting or determining the level of serine phosphorylated Giα1 and/or Giα3 proteins in the cell sample, and/or determining a ratio between the response to Giα protein stimulation and the response to Gsα protein stimulation from a biological sample from the subject. Kits for predicting the risk of an AIS subject for developing a severe scoliosis, for predicting the subject's responsiveness to bracing treatment, and for identifying therapeutically useful compounds are also provided.

### CROSS REFERENCE TO RELATED APPLICATIONS

This application is a divisional application of U.S. patent application Ser. No. 14/898,766, which is a National Phase Entry of PCT application Serial No PCT/CA2014/050562 filed on Jun. 16, 2014 and published in English under PCT Article 21(2), which itself claims benefit of U.S. provisional application Ser. No. 61/835,839, filed on Jun. 17, 2013. All documents above are incorporated herein in their entirety by reference.

### FIELD OF THE INVENTION

The present invention relates to markers of scoliosis and scoliosis progression. More particularly, it relates to Gi protein phosphorylation as marker for scoliosis and scoliosis progression, methods of increasing GiPCR signaling in scoliotic subjects and uses thereof to stratify scoliotic patients and predict the risk of developing scoliosis and methods of increasing GiPCR signaling in scoliotic subjects.

### REFERENCE TO SEQUENCE LISTING

Pursuant to 37 C.F.R. 1.821(c), a sequence listing is submitted herewith as an ASCII compliant text file named 14033_168_ST25.txt, that was created on Apr. 20, 2018 and having a size of 58 kilobytes. The content of the aforementioned file is hereby incorporated by reference in its entirety.

### BACKGROUND OF THE INVENTION

Idiopathic Scoliosis is a spine deformity of unknown cause generally defined as a lateral curvature greater than 10 degrees accompanied by a vertebral rotation¹. Adolescent Idiopathic Scoliosis (AIS) is one of the most frequent childhood deformities worldwide, characterized by a 3D spinal deformity with unknown cause, and represents both an immediate medical challenge and a chronic condition affecting individuals throughout their lives. It is the most common orthopedic condition requiring surgery in adolescents and affects 4% of this population. This condition is most commonly diagnosed between the ages of 9 to 13 years^{2,3,4}. The diagnosis is primarily of exclusion and is made only after ruling out other causes of spinal deformity such as vertebral malformation, neuromuscular or syndromic disorders. Traditionally, the trunkal asymmetry is revealed by Adams forward bending test and measured with scoliometer during physical examination⁵. The diagnosis can then be confirmed by radiographic observation of the curve and the angle measurement using the Cobb method⁶.

Once diagnosed, the primary concern for physicians in managing scoliotic children is whether the curve will progress. Indeed, the curve progression is often unpredictable and is more frequently observed among girls than in boys⁷. If untreated, the curve can progress dramatically, creating significant physical deformity and even cardiopulmonary problems. These manifestations become life threatening when the curve exceeds 70 degrees^{8,9}. The current treatment options to prevent or stop curve progression include bracing and surgery. In general, bracing is recommended for curves between 25 and 40 degrees, while surgery is reserved for curves greater than 45 degrees or curves that are unresponsive to bracing. Today in the United States there are approximately one million children between ages 10 and 16 with some degree of IS. Approximately 10% of children diagnosed with idiopathic scoliosis have curve progression requiring corrective surgery¹⁰. About 29,000 scoliosis surgeries are done every year in North America, resulting in significant psychological and physical morbidity. (Goldberg M S, Mayo N E, Poitras B et al. The Ste-Justine Adolescent Idiopathic Scoliosis Cohort Study. Part I: Description of the study. Spine 1994; 19:1551-61; Poitras B, Mayo N E, Goldberg M S et al. The Ste-Justine Adolescent Idiopathic Scoliosis Cohort Study. Part IV: Surgical correction and back pain. Spine 1994; 19:1582-8).

Currently, there is no proven method or test available to identify subjects at risk of developing IS to predict which affected individuals require treatment to prevent or stop progression of the disease so that appropriate treatment can be early provided and prevent surgical complications and cardiac and/or respiratory problems. (Weinstein S L, Dolan L A, Cheng J C et al. Adolescent idiopathic scoliosis. Lancet 2008; 371:1527-37).

Therefore, the application of current treatments, such as bracing or surgical correction, is delayed until a significant deformity is detected or until a significant progression is clearly demonstrated, resulting in a delayed, less than optimal treatment and often important psychological sequels (Society SR. Morbidity & Mortality Committee annual Report 1997).

Currently, in order to detect the deformity, diagnosed children are subjected to multiple radiographs over several years, usually until they reach skeletal maturity. It is estimated that the typical patients with scoliosis will have approximately 22 radiological examinations over a 3-year period¹¹. There are potential risks in multiple radiographic examinations. For this reason also, alternative approaches that could allow performing the prognosis of idiopathic scoliosis are strongly desirable.

The major limitation in developing prognostic tests that could facilitate treatment choices for patients is the heterogeneous nature of AIS. At the clinical level, the heterogeneity of AIS is clearly illustrated by the variability of curve patterns, localisations and curve magnitude even in families with multiple affected members.

In absence of reliable AIS phenotypes, there is a need to understand better the molecular changes associated with disease onset and spinal deformity progression. Molecular definition of disease is rapidly replacing traditional pathology-based disease descriptions in part because of its utility in identifying the optimal treatment regimen for patients.

To this effect, the existence of a differential melatonin signaling dysfunction was reported among AIS patients leading to their stratification into three functional groups or biological endophenotypes (Moreau et al., 2004); (Azeddine et al., 2007); (Letellier et al., 2008) and WO2003/073102 to Moreau. More particularly, AIS patients were stratified into three functional groups (FG1, FG2 and FG3) representing distinct biological endophenotypes. With this approach, the scoliotic patients and children more at risk of developing scoliosis are less responsive to Gi protein stimulation when compared with healthy control subjects, and the classification is based on the percentage of degree of reduction relative to control group. The classification ranges were fixed between 10 and 40% for FG3, 40 and 60% for FG2 and 60 and 90% for FG1.

More recently, using the cellular dielectric spectrometry (CDS) technique, which is a label-free method for the functional evaluation of G proteins and endogenous receptors coupled to those proteins (Verdonk et al., 2006), it was found that the cellular response following melatonin receptor stimulation by melatonin was mainly Gi-dependent in normal osteoblasts and was reduced to different extents in osteoblasts derived from AIS patients (Akoume et al., 2010). Approximately 33% of asymptomatic children diagnosed with a defective Gi protein function have developed scoliosis many years later (Akoume et al., 2010).

Early detection/prognosis of scoliosis is not only critical to successful and less invasive clinical outcomes but broadens the range of treatment options for clinicians. Indeed, improving patients' stratification and disease staging represent key steps to select AIS patients for minimally invasive surgeries before their spinal deformity is too advanced.

The present description refers to a number of documents, the content of which is herein incorporated by reference in their entirety.

### SUMMARY OF THE INVENTION

The present invention provides the clinical evidence that a differential disruption of Gi alpha subunits occurs in AIS and demonstrate that such impairment is caused by a serine phosphorylation of distinct Gi isoforms leading to the classification of AIS patients into three biological endophenotypes representing inheritable traits. Heritability was clearly demonstrated with the detection of the same endophenotype in all family members affected by scoliosis. Evaluation of the clinical outcomes of AIS patients according to their biological endophenotypes reveals in two distinct cohorts (Canadians and Italians) that AIS patients classified in FG2 endophenotype are more susceptible to developing severe scoliosis, while those in FG1 endophenotype present a much lower risk of disease progression.

In mechanistic in vitro studies, it was further observed that hypofunctionality of Gi proteins occurring in AIS led to (a) a systemic and generalized signaling defect perturbing all Gi-coupled receptors differentially in different cell types, (b) an enhancement of Gs-coupled receptor signaling while Gq-coupled receptor signal transduction was not compromised, and (c) pharmacological inhibition of selected kinases rescued or improved Gi-signaling impairment at the cellular level and such response varied in function of each biological endophenotype. The signaling defect is due to selective phosphorylation of Gi alpha subunit isoforms in each group involving distinct kinases, namely: FG3 has a Giα1 without phosphorylated serines and serine-phosphorylated Giα2 and Giα3; FG2 has a Giα3 without phosphorylated serines and serine-phosphorylated Giα1 and Giα2; and FG1 has serine-phosphorylated Giα1, Giα2 and Giα3.

These findings provide the evidence that a differential disruption of Gi-signaling underscores AIS pathogenesis and represents a rational basis for the development of innovative prognostic tools and pharmacological therapies.

More specifically, in accordance with the present invention, there is provided a method of stratifying a subject having adolescent idiopathic scoliosis (AIS) comprising: (i) providing a cell sample isolated from the subject; and (ii) (a) determining the serine phosphorylation of Giα1 in the cell sample; (b) determining the serine phosphorylation of Giα3 in the cell sample; (c) determining the difference (Δ) between responses to Giα and Gsα protein stimulation in the cell sample; or (d) any combination of (a) to (c); whereby the results of the detecting step enables the stratification of the subject having AIS as belonging to an AIS subclass.

In accordance with another aspect of the present invention, there is provided a method for predicting the risk for developing a severe scoliosis in a subject comprising: (i) providing a cell sample isolated from the subject; (ii) (a) determining the serine phosphorylation of Giα3 protein in the cell sample; (b) determining the difference (Δ) between responses to Giα and Gsα protein stimulation in the cell sample; and/or (c) determining the GiPCR response to an agonist in the cell sample, wherein (a) an absence of serine phosphorylation in Giα3 protein; (b) a determination that the Giα/Gsα ratio is between about 0.5 and 1.5; and/or (c) a GiPCR response in the cell sample lower than that in a control sample by about 40 to 60%, is indicative that the subject is at risk for developing a severe scoliosis.

In a specific embodiment, the subject is a subject diagnosed with a scoliosis.

In a specific embodiment, the subject is likely to develop a scoliosis. In another specific embodiment, the scoliosis is adolescent idiopathic scoliosis. In another specific embodiment, method of is in vitro. In another specific embodiment, said cell sample comprises osteoblasts, myoblasts and/or peripheral blood mononuclear cells (PBMC). In another specific embodiment, said cell sample comprises PBMCs. In another specific embodiment, said cell comprises lymphocytes.

In accordance with another aspect of the present invention, there is provided a method of increasing GiPCR signaling in cells of a subject in need thereof (e.g., diagnosed with a scoliosis or a likely to develop scoliosis) comprising administering to the subject an effective amount of: (i) an inhibitor of PKA; or (ii) if the subject's cells have serine phosphorylated Gαi1, Gαi2 and Gαi3 proteins, an inhibitor of (a) PKC; (b) CaMK1 or 4; (c) CK; or (d) any combination of at least two of (a) to (c); (iii) if the subjects cells have an absence of serine phosphorylation on Gαi3, an inhibitor of CaMK2; or (iv) if the subjects cells have an absence of serine phosphorylation on Gαi1, an inhibitor of CK, whereby the GiPCR signaling is increased in the subject's cells.

In accordance with another aspect of the present invention, there is provided a use of (i) an inhibitor of PKA; or (ii) if the subject's cells have serine phosphorylated Gαi1, Gαi2 and Gαi3 proteins, an inhibitor of (a) PKC; (b) CaMK1 or 4; (c) CK; or (d) any combination of at least two of (a) to (c); (iii) if the subjects cells have an absence of serine phosphorylation on Gαi3, an inhibitor of CaMK2; or (iv) if the subjects cells have an absence of serine phosphorylation on Gαi1, an inhibitor of CK, for increasing GiPCR signaling in cells of a subject in need thereof (e.g., diagnosed with a scoliosis or a likely to develop scoliosis) or for manufacturing a medicament for increasing GiPCR signaling in cells of a subject in need thereof (e.g., diagnosed with a scoliosis or a likely to develop scoliosis).

In accordance with another aspect of the present invention, there is provided a composition for use in increasing GiPCR signaling in cells of a subject in need thereof (e.g., diagnosed with a scoliosis or a likely to develop scoliosis) comprising: (i) an inhibitor of PKA; or (ii) if the subject's cells have serine phosphorylated Gαi1, Gαi2 and Gαi3 proteins, an inhibitor of (a) PKC; (b) CaMK1 or 4; (c) CK; or (d) any combination of at least two of (a) to (c); (iii) if the subjects cells have an absence of serine phosphorylation on Gαi3, an inhibitor of CaMK2; or (iv) if the subjects cells have an absence of serine phosphorylation on Gαi1, an inhibitor of CK.

In accordance with another aspect of the present invention, there is provided a composition comprising: (i) an inhibitor of PKA; or (ii) if the subject's cells have serine phosphorylated Gαi1, Gαi2 and Gαi3 proteins, an inhibitor of (a) PKC; (b) CaMK1 or 4; (c) CK; or (d) any combination of at least two of (a) to (c); (iii) if the subjects cells have an absence of serine phosphorylation on Gαi3, an inhibitor of CaMK2; or (iv) if the subjects cells have an absence of serine phosphorylation on Gαi1, an inhibitor of CK. In specific embodiments of the present invention, the compositions further comprise a pharmaceutically acceptable carrier.

In accordance with another aspect of the present invention, there is provided a kit for stratifying a subject having adolescent idiopathic scoliosis (AIS) comprising: (a) a ligand for detecting an absence of serine phosphorylation on Giα1 in the cell sample; (b) a ligand for detecting an absence of serine phosphorylation on Giα3 in the cell sample; (c) ligands for detecting Giα and Gsα; or (d) any combination of (a) to (c).

In accordance with another aspect of the present invention, there is provided a kit for predicting the risk for developing a severe scoliosis in a subject comprising: (a) a ligand for detecting an absence of serine phosphorylation on Giα3; (b) ligands for detecting Giα and Gsα; or (c) a combination of (a) and (b).

In accordance with another aspect of the present invention, there is provided a kit for increasing GiPCR signaling in cells of a subject in need thereof comprising: (a) an inhibitor of PKA; (b) an inhibitor of PKC; (c) an inhibitor of CaMK1 or 4; (d) an inhibitor of CK; (e) an inhibitor of CaMK2; or (f) any combination of at least two of (a) and (e).

In specific embodiments of the invention, the inhibitor of PKA is an inhibitor of PKA-γ2. In another specific embodiment, the inhibitor of PKA is H89. In another specific embodiment, the inhibitor of PKC is an inhibitor of PKC-η or PKC-ε. In a specific embodiment, the inhibitor of PKC is Gö6983. In a specific embodiment, the inhibitor of CaMK1 is CaMk1δ. In a specific embodiment, the inhibitor of CaMK1 or CaMK4 is STO609. In a specific embodiment, the inhibitor of CK is an inhibitor of CK2. In a specific embodiment, the inhibitor of CK is D4476. In a specific embodiment, the inhibitor of CaMK2 is STO609 or KN93. In a specific embodiment, the subject in need thereof is a subject diagnosed with a scoliosis. In a specific embodiment, the subject in need thereof is likely to develop a scoliosis. In a specific embodiment, the scoliosis is adolescent idiopathic scoliosis. In a specific embodiment, the method is in vitro.

In accordance with another aspect of the present invention, there is provided a method of selecting an agent as a potential candidate for the reduction or prevention of scoliosis comprising contacting a candidate agent with a cell expressing (a) PKA; (b) PKC, (c) CaMK1, (d) CaMK4; (e) CK; (f) CaMK2; wherein when the expression or activity of any one of (a) to (f) is decreased, the candidate agent is selected.

In specific embodiments of the invention, PKA is PKA-γ2. In other specific embodiments of the invention PKC is PKC-η or PKC-ε. In other specific embodiments of the invention, CaMK1 is CaMK1δ. In other specific embodiments of the invention CK is a CK2.

In some aspects, the present invention relates to a method of stratifying a subject having or at risk for developing adolescent idiopathic scoliosis (AIS), the method comprising: (i) providing a cell sample isolated from the subject; (ii) detecting or determining from the cell sample the subject's Giα protein serine phosphorylation profile and/or the degree of imbalance in response to Giα and Gsα protein stimulation; and (iii) stratifying the subject into a clinically useful AIS subclass based on the subject's Giα protein serine phosphorylation profile and/or the degree of imbalance in response to Giα and Gsα protein stimulation.

In some embodiments, the present above mentioned method comprises: (a) detecting or determining the serine phosphorylation of Giα1 in the cell sample; (b) detecting or determining the serine phosphorylation of Giα3 in the cell sample; (c) detecting or determining a ratio (Giα/Gsα response ratio) or difference (Δ) between the response to Giα protein stimulation and the response to Gsα protein stimulation in the cell sample; or (d) any combination of (a) to (c).

In some embodiments, the above mentioned method further comprises stratifying the subject as belonging to: (1) a first AIS subclass characterized by: (a) elevated levels of serine-phosphorylated Giα1 and Giα3 proteins as compared to levels corresponding to those of a control; and/or (b) a Giα/Gsα response ratio below about 0.5; (2) a second AIS subclass characterized by: (a) elevated levels of serine-phosphorylated Giα1 but not of serine-phosphorylated Giα3 protein, as compared to levels corresponding to that of a control; and/or (b) a Giα/Gsα response ratio between about 0.5 and 1.5; or (3) a third AIS subclass characterized by: (a) elevated levels of serine-phosphorylated Giα3 protein but not of serine-phosphorylated Giα1 protein as compared to levels corresponding to those of a control; and/or (b) a Giα/Gsα response ratio above about 1.5.

In some embodiments, the above mentioned method further comprises detecting or determining the serine phosphorylation of Giα2 protein in the cell sample, wherein elevated levels of serine-phosphorylated Giα2 protein are detected, as compared to levels corresponding to that of a control.

In some embodiments of the above mentioned methods, (1) subjects belonging to the first AIS subclass have a low risk of severe AIS progression; (2) subjects belonging to the second AIS subclass have a high risk for severe AIS progression; and (3) subjects belonging to the third AIS subclass have a moderate risk for severe AIS progression.

In some aspects, the present invention relates to a method for predicting the risk for developing a severe scoliosis in a subject having or at risk for developing scoliosis, the method comprising: (i) providing a cell sample isolated from the subject; (ii) detecting or determining: (a) the serine phosphorylation of Giα3 protein in the cell sample; (b) responses to Giα and Gsα proteins stimulation in the cell sample; and/or (c) the GiPCR response to an agonist in the cell sample; (iii) determining that the subject is at risk for developing a severe scoliosis when: (a) serine-phosphorylated Giα3 protein in the cell sample is not detected, or is not elevated as compared to levels corresponding to those of a control; (b) a ratio of the response to Giα protein stimulation to the response to Gsα protein stimulation (Giα/Gsα response ratio) in the cell sample is between about 0.5 and 1.5; and/or (c) a GiPCR response in the cell sample lower than that in a control sample by about 40 to 60% is detected.

In some embodiments of the above mentioned methods, step (ii) further comprises detecting or determining the serine phosphorylation of Giα1 and/or Giα2 protein in the cell sample, wherein the subject is at risk for developing a severe scoliosis when the level of the subject's serine-phosphorylated Giα1 or Giα2 protein is elevated as compared to levels corresponding to those of a control.

In some aspects, the present invention relates to a method for predicting the responsiveness of a subject having scoliosis to bracing treatment, the method comprising: (i) providing a cell sample isolated from the subject; and (ii) detecting or determining: (a) the serine phosphorylation of Giα1 in the cell sample; (b) the serine phosphorylation of Giα3 in the cell sample; (c) responses to Giα and Gsα proteins stimulation in the cell sample; or (d) any combination of (a) to (c); (iii) determining that the subject is likely to be responsive to bracing treatment when: (a) elevated levels of serine-phosphorylated Giα3 protein but not of serine-phosphorylated Giα1 protein is detected, as compared to levels corresponding to those of a control; and/or (b) a ratio of the response to Giα protein stimulation to the response to Gsα protein stimulation (Giα/Gsα response ratio) in the cell sample is above about 1.5.

In some embodiments, the above mentioned subject is a subject diagnosed with a scoliosis. In some embodiments, the scoliosis is adolescent idiopathic scoliosis (AIS).

In some embodiments, the above mentioned method is in vitro.

In some embodiments, the above mentioned cell sample comprises osteoblasts, myoblasts and/or peripheral blood mononuclear cells (PBMC). In some embodiments, the above mentioned cell sample comprises PBMCs. In some embodiments, the above mentioned PBMCs comprise lymphocytes.

In some embodiments, the above mentioned the detecting or determining the serine phosphorylation of Giα1 and/or Giα3 protein(s) in the cell sample comprises isolating Giα1 and/or Giα3 protein(s) from the cell sample and contacting the isolated Giα1 and/or Giα3 protein(s) with an anti-phosphoserine antibody.

In some aspects, the present invention relates to a method of increasing GiPCR signaling in cells of a subject having or at risk for developing scoliosis, the method comprising administering to the subject an effective amount of: (i) an inhibitor of PKA; (ii) an inhibitor of (a) PKC; (b) CaMK1 or 4; (c) CK; or (d) any combination of at least two of (a) to (c), if the subject's cells have elevated levels of serine phosphorylated Gαi1, Gαi2 and Gαi3 proteins as compared to levels corresponding to those of a control; (iii) an inhibitor of CaMK2, if the subject's cells have levels of serine phosphorylated Gαi3 protein that are not elevated as compared to levels corresponding to those of a control; or (iv) an inhibitor of CK, if the subject's cells have levels of serine phosphorylated Gαi1 protein that are not elevated as compared to levels corresponding to those of a control, whereby the GiPCR signaling is increased in the subject's cells.

In some embodiments, the above mentioned inhibitor of PKA is an inhibitor of PKA-γ2. In some embodiments, the above mentioned inhibitor of PKA is H89. In some embodiments, the above mentioned inhibitor of PKC is an inhibitor of PKC-η or PKC-ε. In some embodiments, the above mentioned inhibitor of PKC is Gö6983. In some embodiments, the above mentioned inhibitor of CaMK1 is CaMK1δ. In some embodiments, the above mentioned inhibitor of CaMK1 or CaMK4 is STO609. In some embodiments, the above mentioned inhibitor of CK is an inhibitor of CK2. In some embodiments, the above mentioned inhibitor of CK is D4476. In some embodiments, the above mentioned inhibitor of CaMK2 is STO609 or KN93.

In some embodiments, the above mentioned subject in need thereof is a subject diagnosed with a scoliosis. In some embodiments, the above mentioned subject in need thereof is likely to develop a scoliosis. In some embodiments, the above mentioned scoliosis is adolescent idiopathic scoliosis (AIS).

In some embodiments, the above mentioned method is in vitro.

In some aspects, the present invention relates to the use of an inhibitor as defined above for increasing GiPCR signaling, or in the preparation of a medicament for increasing GiPCR signaling, in cells of a subject having or at risk for developing scoliosis.

In some embodiments, the above mentioned subject in need thereof is a subject diagnosed with a scoliosis. In some embodiments, the above mentioned the subject in need thereof is likely to develop a scoliosis. In some embodiments, the above mentioned the scoliosis is adolescent idiopathic scoliosis (AIS).

In some aspects, the present invention relates to a kit for stratifying a subject having or at risk for developing adolescent idiopathic scoliosis (AIS), the kit comprising: (a) a ligand for detecting the level of serine-phosphorylated Giα1 protein in a cell sample from the subject; (b) a ligand for detecting the level of serine-phosphorylated Giα3 protein in a cell sample from the subject; (c) ligands for detecting responses to Giα and Gsα proteins stimulation in the cell sample; or (d) any combination of (a) to (c).

In some embodiments, the above mentioned kit further comprises a ligand for detecting the level of serine-phosphorylated Giα2 protein.

In some aspects, the present invention relates to a kit for predicting the risk for developing a severe scoliosis in a subject having or at risk for developing scoliosis, the kit comprising: (a) a ligand for detecting the level of serine-phosphorylated Giα3 protein in a cell sample from the subject; (b) ligands for detecting responses to Giα and Gsα proteins stimulation in a cell sample from the subject; or (c) a combination of (a) and (b).

In some aspects, the present invention relates to a kit for increasing GiPCR signaling in cells of a subject in need thereof, the kit comprising: (a) an inhibitor of PKA; (b) an inhibitor of PKC; (c) an inhibitor of CaMK1 or 4; (d) an inhibitor of CK; (e) an inhibitor of CaMK2; or (f) any combination of at least two of (a) to (e).

In some embodiments, the above mentioned inhibitor of PKA is an inhibitor of PKA-γ2. In some embodiments, the above mentioned inhibitor of PKA is H89. In some embodiments, the above mentioned inhibitor of PKC is an inhibitor of PKC-η or PKC-ε. In some embodiments, the above mentioned inhibitor of PKC is Gö6983. In some embodiments, the above mentioned inhibitor of CaMK1 is CaMK1δ. In some embodiments, the above mentioned inhibitor of CaMK1 or CaMK4 is STO609. In some embodiments, the above mentioned inhibitor of CK is an inhibitor of CK2. In some embodiments, the above mentioned inhibitor of CK is D4476. In some embodiments, the above mentioned inhibitor of CaMK2 is STO609 or KN93.

In some aspects, the present invention relates to a method of selecting an agent as a potential candidate for the treatment or prevention of scoliosis, the method comprising contacting a candidate agent with a cell expressing: (a) PKA; (b) PKC; (c) CaMK1; (d) CaMK4; (e) CK; or (f) CaMK2; and selecting the candidate agent when the expression or activity of any one of (a) to (f) is decreased. In some embodiments, the above mentioned PKA is PKA-γ2. In some embodiments, the above-mentioned PKC is PKC-η or PKC-ε. In some embodiments, the above mentioned CaMK1 is CaMK1δ. In some embodiments, the above mentioned CK is a CK2.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows representative pedigrees of families with a positive history of AIS. The circles represent females and the squares represent males. Filled symbols indicate affected individuals and the indicated numbers correspond to the individuals (case numbers) listed in FIG. 2.

FIG. 2 presents AIS subjects functional groups and clinical data of affected members from studied families.

FIG. 3A-FIG. 3E show that response to melatonin is reduced but the functionality of the melatonin receptor is not impaired in AIS. (FIG. 3A) Comparison of concentration-response curves for melatonin between control and AIS functional groups. Data were normalised to maximal response (% maximal response) in cells from control subjects. (FIG. 3B) Comparison of response to melatonin and its analogues. Cells were stimulated with the same concentration (1 μM) of melatonin, iodomelatonin or phenylmelatonin. The impedance represented in y-axis as dZiec, indicates the resistance (ohms) of the cells toward the electric current applied by the Cellkey™ apparatus and represents the integrated cellular response. (FIGS. 3C-D) Inhibition curves for response to melatonin following a treatment of (FIG. 3C) 4 h with G-Protein antagonist peptide (GPAnt-2) and (FIG. 3D) 16 hours with pertussis toxin. (FIG. 3E) EC₅₀ and IC₅₀ values of melatonin and GPAnt-2 in each functional group. Cellular responses shown on the y-axis in (FIG. 3A) to (FIG. 3E) were measured by CDS. Data are expressed as mean (±SE) of n=12 patients per group. * P<0.05, **P<0.01, ***P<0.001, vs control group.

FIG. 4A-FIG. 4G show the AIS functional groups are distinguished by the degree of response to various specific agonists of Gi-coupled receptors in osteoblasts. (FIG. 4A, FIG. 4B, FIG. 4C, FIG. 4D, FIG. 4E, and FIG. 4F). The cellular response of osteoblasts to increasing concentrations of agonists targeting various Gi-coupled receptors was measured by cellular dielectric spectroscopy (CDS). Agonists and targeted receptors are indicated in the left corner of each panel. Dose-response data were normalised to maximal response in cells from control subjects (% maximum response shown on the y-axis and measured by CDS). (FIG. 4G) EC₅₀ values of tested compounds in each functional group. Data are expressed as mean (±SE) of n=12 patients per group. * P<0.05, **P<0.01, ***P<0.001, vs control group.

FIG. 5A-FIG. 5G show inhibition curves of GPAnt-2 on response to various selective agonists of Gi-coupled receptors. (FIG. 5A, FIG. 5B, FIG. 5C, FIG. 5D, FIG. 5E, and FIG. 5F) Osteoblasts from control subjects or AIS patients of different groups were pre-incubated with varying concentrations of GPAnt-2 for 4 h prior stimulation with 1 μM of specific synthetic agonist. The cellular response of osteoblasts to agonist stimulation was measured by CDS. The tested agonists and targeted receptors are indicated in left corner of each panel. Dose-response data were normalised to maximal response in cells from control subjects (% maximum response shown on the y-axis and measured by CDS). (FIG. 5G) Table of IC₅₀ values GPAnt-2 for each tested compound in each functional group. Data are expressed as mean (±SE) of n=12 patients per group. * P<0.05, **P<0.01, ***P<0.001, vs control group.

FIG. 6A-FIG. 6F show inhibition curves of PTX on response to various selective agonists of Gi-coupled receptors. (FIG. 6A, FIG. 6B, FIG. 6C, FIG. 6D, FIG. 6E, and FIG. 6F) Osteoblasts from control subjects or AIS patients of different groups were pre-incubated with varying concentrations of PTX for 16 h prior to stimulation with 1 μM of specific synthetic agonist. The tested agonists and targeted receptors are indicated in left corner of each panel. Data were normalised to maximal response in cells from control subjects. Data are expressed as mean±SE of n=12 patients per group. * P<0.05, **P<0.01, ***P<0.001, vs control group.

FIGS. 7A-G show the AIS functional groups are distinguished by the degree of response to various specific agonists of Gi-coupled receptors in myoblasts. (FIG. 7A, FIG. 7B, FIG. 7C, FIG. 7D, FIG. 7E, and FIG. 7F). The cellular response of myoblasts to increasing concentrations of agonists targeting various Gi-coupled receptors was measured by CDS. Agonists and targeted receptors are indicated in the left corner of each panel. Dose-response data were normalised to maximal response in cells from control subjects (% maximum response shown on the y-axis and measured by CDS). (FIG. 7G) EC₅₀ values of tested compounds in each functional group. Data are expressed as mean (±SE) of n=12 patients per group. * P<0.05, **P<0.01, ***P<0.001, vs control group.

FIG. 8A-FIG. 8G show the AIS functional groups are distinguished by the degree of response to various specific agonists of Gi-coupled receptors in PBMCs (FIG. 8A, FIG. 8B, FIG. 8C, FIG. 8D, FIG. 8E, and FIG. 8F). The cellular response of PBMCs to increasing concentrations of agonists targeting various Gi-coupled receptors was measured by CDS. Agonists and targeted receptors are indicated in the left corner of each panel. Dose-response data were normalised to maximal response in cells from control subjects (% maximum response shown on the y-axis and measured by CDS). (FIG. 8G) EC₅₀ values of tested compounds in each functional group. Data are expressed as mean (±SE) of n=12 patients per group. * P<0.05, **P<0.01, ***P<0.001, vs. control group.

FIG. 9A-FIG. 9F show the functional status of Gs and Gq proteins in osteoblasts from control and AIS functional groups. The functionality of Gs protein was evaluated by challenging cells with (FIG. 9A) Isoproterenol; or (FIG. 9B) Desmopressin. (FIG. 9C) The difference between response to Gi and Gs stimulation was calculated at various concentrations, and the functionality of Gq was assessed by challenging cells with Bradykinin (FIG. 9D) or Endothelin-1 (FIG. 9E). Receptor subtype targeted by the indicated agonist appears in parentheses. Dose-response data were normalised to maximal response in cells from control subjects (% maximum response shown on the y-axis and measured by CDS). (FIG. 9F) EC₅₀ values of tested compounds in each functional group. Cellular response in (A) to (F) was measured by CDS. Data are expressed as mean (±SE) of n=12 patients per group. * P<0.05, **P<0.01, ***P<0.001, vs control group.

FIG. 10A-FIG. 10B show that expression of Gi and Gs mRNA and proteins is similar between AIS functional groups. (FIG. 10A) Total RNA was extracted from osteoblasts and qPCR was used to compare mRNA expression levels of Gi₁, Gi₂, Gi₃ and Gs genes in control relative to the AIS functional groups. β-actin was used as internal control. (FIG. 10B) Lysates were obtained from osteoblasts of each functional group. Equal amounts of proteins (40 μg) of each lysate were resolved by 10% SDS-PAGE and immunoblotted for Gi1, Gi2, Gi3 or Gs proteins. β-actin was used as internal control.

FIG. 11A-FIG. 11C show the differential phosphorylation patterns of Gi protein isoforms in AIS functional groups. Whole osteoblast cells from control or AIS patients were subjected to immunoprecipitation with antibody recognizing (FIG. 11A) Gi₁, (FIG. 11B) Gi₂ or (FIG. 11C) Gi₃, and these precipitates were resolved by 10% SDS-PAGE and immunoblotted for phospho-serine/threonine specific antibody. Representative immunoblots from a single experiment are shown in the inserts. The bands were quantified by densitometric scanning. Values are expressed as mean±SE, of n=6 patients per group. * P<0.05, **P<0.01, ***P<0.001, vs control group. Insert despites a typical immunodetectable phosphorylation profile of corresponding Gi protein isoforms in functional groups.

FIG. 12A-FIG. 12H show the differential effects of Gi and Gs siRNA on response to Gi stimulation in AIS functional groups. Osteoblasts from (FIG. 12A) control subjects, (FIG. 12B) FG3, (FIG. 12C) FG2; and (FIG. 12D) FG1 were transfected with Gi₁, Gi₂, Gi₃ or Gs siRNA alone or in combination, or with scrambled siRNA, as indicated in Example 1—Materials and Methods. Efficiency of siRNA was verified with qPCR in each functional group (FIG. 12E to FIG. 12H) 48 hours after transfection, and response to Gi stimulation was measured by CDS and evaluated by challenging cells with Apelin-17, LPA or Somatostatin. Results from a single representative subject for each group are presented. Data were normalised to response in cells transfected with scrambled siRNA (% response shown on the y-axis and measured by CDS), and are expressed as mean±SE, of n=6 patients in each functional group. * P<0.05, **P<0.01, ***P<0.001, vs. control group.

FIG. 13A-FIG. 13B show the effect of various serine/threonine kinase inhibitors on response to Gi stimulation in osteoblasts from control and AIS functional groups. Cells were treated with Protein kinase A (PKA) inhibitor H89 (5 μM), Protein kinase C (PKC) inhibitor Gö6983 (5 μM), Ca²⁺/calmodulin-dependent protein kinase (CaMK) (1,2,4) inhibitor STO-609 acetate (5 μM), Ca²⁺/calmodulin-dependent protein kinase II (CaMK-2) inhibitor KN93 (5 μM), Casein kinase 1 (CK1) inhibitor D4476 (5 μM) or vehicle for 1 hour prior to stimulation with (FIG. 13A) LPA (10⁻⁶ M) or (FIG. 13B) Somatostatin (10⁻⁶ M). Response to Gi stimulation was measured by CDS. Data were normalised to response in osteoblasts treated with vehicle (% response in presence of vehicle shown on the y-axis and measured by CDS), and are expressed as mean±SE, of n=12 patients in each functional group. * P<0.05, **P<0.01, ***P<0.001, vs control group.

FIG. 14A-FIG. 14B show mRNA and protein expression of various serine/threonine kinases in AIS. (FIG. 14A) Total RNA was extracted from osteoblasts and qPCR was used to compare mRNA expression levels of PKC, PKA, CaMK and CK isoforms in control relative to the AIS functional groups. β-actin was used as internal control. (FIG. 14B) Lysates were obtained from osteoblasts of each functional group. Equal amounts of proteins (40 μg) of each lysate were resolved by 10% SDS-PAGE and immunoblotted for proteins of indicated kinase isoforms.

FIG. 15A-FIG. 15B show mRNA expression of various PKC serine/threonine kinase isoforms in AIS. Total RNA was extracted from osteoblasts and qPCR was used to compare mRNA expression levels of (FIG. 15A) various PKC isoforms (i.e., PKC-α, PKC-β, PKC-δ, PKC-I, PKC-γ, PKC-θ and PKC-ζ) and (FIG. 15B) various isoforms of CaMK (i.e., CaMK1, CaMK1γ, CaMK2α, CaMK2β, CaMK2δ, CaMK2γ, CaMK2N1, CaMK2N2 and CaMK4) in control relative to the AIS functional groups. β-actin was used as internal control.

FIG. 16A-FIG. 16B show mRNA expression of various PKA and CK serine/threonine kinase isoforms in AIS. Total RNA was extracted from osteoblasts and qPCR was used to compare mRNA expression levels of various isoforms of PKA and CK (i.e., (FIG. 16A) PKA-α1, PKA-α2, PKA-β1, PKAβ2, PKA-cβ, PKA-cγ, PKA-γ1 and PKA-γ3; and (FIG. 16B) CK1α, CK1β, CK1δ, CK1ε, CK1γ1, CK1γ2, CK1γ3, CK2α1, CK2α2, CK2β) in control relative to the AIS functional groups. β-actin was used as internal control.

FIG. 17 shows a sequence alignment between the amino acid sequences of Giα1 isoforms 1 (SEQ ID NO: 78) and 2 (amino acid residues 53 to 354 SEQ ID NO: 78).

FIG. 18 shows a multiple sequence alignment between the amino acid sequences of Giα2 isoforms 1-6. Amino acid sequence of Giα2-Isoform-1 corresponds to SEQ ID NO: 82; of Giα2-Isoform-2 corresponds to SEQ ID NO: 84; of Giα2-Isoform-3 corresponds to SEQ ID NO: 86; of Giα2-Isoform-4 corresponds to SEQ ID NO: 88; of Giα2-Isoform-5 corresponds to SEQ ID NO: 90; and of Giα2-Isoform-6 corresponds to SEQ ID NO: 92.

FIG. 19A-FIG. 19B show a sequence alignment between the amino acid sequences of Giα1 isoforms 1 and 2, Giα2 isoforms 1-6, and Giα3. Amino acid sequence of Giα1-Isoform-1 corresponds to SEQ ID NO: 78; of Giα1-Isoform-2 corresponds to SEQ ID NO: 80; of Giα2-Isoform-1 corresponds to SEQ ID NO: 82; of Giα2-Isoform-2 corresponds to SEQ ID NO: 84; of Giα2-Isoform-3 corresponds to SEQ ID NO: 86; of Giα2-Isoform-4 corresponds to SEQ ID NO: 88; of Giα2-Isoform-5 corresponds to SEQ ID NO: 90; of Giα2-Isoform-6 corresponds to SEQ ID NO: 92; and of Giα3 corresponds to SEQ ID NO: 94.

### DESCRIPTION OF ILLUSTRATIVE EMBODIMENTS

As used herein the terms “risk of developing scoliosis” refer to a genetic or metabolic predisposition of a subject to develop a scoliosis (i.e., spinal deformity) and/or to develop a more severe scoliosis at a future time (i.e., curve progression). For instance, an increase of the Cobb's angle of a subject (e.g., from 40° to 50°, or from 18° to 25°) is a “development” of scoliosis.

As used herein, the term “severe progression” is an increase of a subject's Cobb's angle to 45° or more, potentially at a younger age.

In accordance with the present invention, subjects having AIS can be stratified into at least three distinct AIS subclasses (FG1, FG2, or FG3) based on their Giα protein phosphorylation profiles and/or the degree of imbalance in the responses to Giα and Gsα protein stimulation. Subjects belonging to a first AIS subclass (FG1) generally have a relatively “low risk” of severe progression—i.e., their risk of developing severe scoliosis is lower than that of subjects belonging to the second (FG2) and third AIS subclasses (FG3). Subjects belonging to a second AIS subclass (FG2) generally have a relatively “high risk” of severe progression—i.e., their risk of developing severe scoliosis is higher than that of subjects belonging to the first (FG1) and third AIS subclasses (FG3). Subjects belonging to a third AIS subclass (FG3) generally have a “moderate risk” of severe progression—i.e., their risk of developing severe scoliosis is higher than that of subjects belonging to the first (FG1) AIS subclass, but less than that of subjects belonging to the second AIS subclass (FG2). It has also been found that subjects belonging to this third AIS subclass (FG3) are more likely to respond to bracing treatment (U.S. provisional application No. 61/879,314).

In an embodiment, the above-mentioned subject is a likely candidate for developing a scoliosis, such as idiopathic scoliosis (e.g., Infantile Idiopathic Scoliosis, Juvenile Idiopathic Scoliosis or Adolescent Idiopathic Scoliosis (AIS)). As used herein, the expressions “likely candidate for developing scoliosis” or “likely to develop scoliosis” include subjects (e.g., children) of which at least one parent has a scoliosis (e.g., adolescent idiopathic scoliosis). Among other factors, age (adolescence), gender and other family antecedents are factors that are known to contribute to the risk of developing a scoliosis and are used to a certain degree to assess the risk of developing a scoliosis. In certain subjects, scoliosis develops rapidly over a short period of time to the point of requiring a corrective surgery (often when the deformity reaches a Cobb's angle≥50°). Current courses of action available from the moment a scoliosis such as AIS is diagnosed (when scoliosis is apparent) include observation (when Cobb's angle is around 10-25°), orthopedic devices (when Cobb's angle is around 25-30°), and surgery (over 45°). A more reliable determination of the risk of progression could enable to 1) select an appropriate diet to remove certain food products identified as contributors to scoliosis; 2) select the best therapeutic agent; and/or 3) select the least invasive available treatment such as postural exercises, orthopedic device, or less invasive surgeries or surgeries without fusions (a surgery that does not fuse vertebra and preserves column mobility). The present invention encompasses selecting the most efficient and least invasive known preventive actions or treatments in view of the determined risk of developing scoliosis.

As used herein the term “subject” is meant to refer to any mammal including human, mouse, rat, dog, chicken, cat, pig, monkey, horse, etc. In a particular embodiment, it refers to a human.

A “subject in need thereof” or a “patient” in the context of the present invention is intended to include any subject that will benefit or that is likely to benefit from an inhibitor of PKA (e.g., PKA-γ2), PKC (e.g., PKC-ε, PKC-η), CaMK1 (e.g., CaMK1δ), CaMK4, CK (e.g., CK2), and/or CaMK2. In an embodiment, a subject in need thereof is a subject diagnosed with a scoliosis (e.g., AIS). In another embodiment, the subject is likely to develop a scoliosis (e.g., AIS) or is at risk of developing scoliosis”.

As used herein the terminology “biological sample” refers to any solid or liquid sample isolated from a living being. In a particular embodiment, it refers to any solid or liquid sample isolated from a human. Without being so limited, it includes a biopsy material, blood, tears, saliva, maternal milk, synovial fluid, urine, ear fluid, amniotic fluid and cerebrospinal fluid. In a specific embodiment it refers to a blood sample. As used herein, the terminology “blood sample” is meant to refer to blood, plasma or serum.

As used herein, the expression “cell sample” refers to a biological sample containing GiPCR-expressing cells obtained from a subject (e.g., a subject having, suspected of having, or at risk of developing AIS).

As used herein the terminology “control sample” is meant to refer to a corresponding sample that does not come from a subject known to have scoliosis or known to be a likely candidate for developing a scoliosis. In methods for determining the risk of developing scoliosis in a subject that is pre-diagnosed with scoliosis, the sample may however also come from the subject under scrutiny at an earlier stage of the disease or disorder.

As used herein the terminology “control” is meant to encompass “control sample”. In certain embodiments, the term “control” also refers to the average or median value obtained following determination of Giα (e.g., 1, 2 and/or 3) protein phosphorylation (e.g., serine phosphorylation) profiles and/or the degree of imbalance in the responses to Giα and Gsα proteins stimulation in a plurality of samples (e.g., samples obtained from several subjects not known to have scoliosis and not known to be a likely candidate for developing scoliosis).

As used herein the term “treating” or “treatment” in reference to scoliosis is meant to refer to at least one of a reduction of Cobb's angle in a preexisting spinal deformity, improvement of column mobility, preservation/maintenance of column mobility, improvement of equilibrium and balance in a specific plan; maintenance/preservation of equilibrium and balance in a specific plan; improvement of functionality in a specific plan, preservation/maintenance of functionality in a specific plan, cosmetic improvement, and combinations of any of the above.

As used herein the term “preventing” or “prevention” in reference to scoliosis is meant to refer to a at least one of a reduction in the progression of a Cobb's angle in a patient having a scoliosis or in an asymptomatic patient, a complete prevention of apparition of a spinal deformity, including changes affecting the rib cage and pelvis in 3D, and a combination of any of the above.

As used herein, the expression “detecting or determining the serine phosphorylation” of a Giα protein (e.g., Giα1, Giα2, and/or Giα3) in the cell sample relates to independently assessing the serine phosphorylation status of the Giα1 and/or Giα3 proteins in a cell sample from a subject. In some embodiments, this assessing can include determining the presence or absence of serine-phosphorylated Giα1, Giα2 and/or Giα3 proteins in the cell sample. In some embodiments, this assessing can include determining the level of expression of serine-phosphorylated Giα1, Giα2 and/or Giα3 proteins in the sample, or the proportion of total Giα1 and/or Giα3 proteins (e.g., both phosphorylated and unphosphorylated) in the cell sample which are serine-phosphorylated. In some embodiments, this assessing involves detecting each of the serine-phosphorylated Giα proteins independently (e.g., using specific anti-Giα1, anti-Giα2, and/or anti-Giα3 antibodies).

Guanine nucleotide binding proteins are heterotrimeric signal-transducing molecules consisting of alpha, beta, and gamma subunits. The alpha subunit binds guanine nucleotide, can hydrolyze GTP, and can interact with other proteins. As used herein, the expression “Giα” or “Giα protein” refers to the alpha subunit of members of the Gi-family of heterotrimeric G proteins. There are several types of Gi alpha subunits, including “Giα1” or “Giα1 protein”, “Giα2” or “Giα2 protein”, and “Giα3” or “Giα3 protein”. As used herein, the expression “Gs” or “Gs protein” refers to the Gs subunit of members of the Gs-family of heterotrimeric G proteins. Examples of Giα and Gs nucleotide and amino acid sequences are shown in the Table below. Unless otherwise indicated, reference to a particular Giα protein (e.g., Giα1, Giα2, Giα3) includes all expressed isoforms of that particular Giα protein.

| Gene | Protein | | Amino acid |
|---|---|---|---|
| (GeneID) | (accession No.) | cDNA sequence | sequence |
| GNAI1 | Giα1 isoform 1 | SEQ ID NO: 77 | SEQ ID NO: 78 |
| (2770) | (NP_002060) | | |
| GNAI1 | Giα1 isoform 2 | SEQ ID NO: 79 | SEQ ID NO: 80 |
| (2770) | (NP_001243343) | | |
| GNAI2 | Giα2 isoform 1 | SEQ ID NO: 81 | SEQ ID NO: 82 |
| (2771) | (NP_002061) | | |
| GNAI2 | Giα2 isoform 2 | SEQ ID NO: 83 | SEQ ID NO: 84 |
| (2771) | (NP_001159897) | | |
| GNAI2 | Giα2 isoform 3 | SEQ ID NO: 85 | SEQ ID NO: 86 |
| (2771) | (NP_001269546) | | |
| GNAI2 | Giα2 isoform 4 | SEQ ID NO: 87 | SEQ ID NO: 88 |
| (2771) | (NP_001269547) | | |
| GNAI2 | Giα2 isoform 5 | SEQ ID NO: 89 | SEQ ID NO: 90 |
| (2771) | (NP_001269548) | | |
| GNAI2 | Giα2 isoform 6 | SEQ ID NO: 91 | SEQ ID NO: 92 |
| (2771) | (NP_001269549) | | |
| GNAI3 | Giα3 | SEQ ID NO: 93 | SEQ ID NO: 94 |
| (2773) | (NP_006487) | | |
| GNAS | Gs | SEQ ID NO: 95 | SEQ ID NO: 96 |
| (2778) | (NM_000516) | | |

As used herein, the expression “detecting or determining responses to Giα and Gsα protein stimulation in the cell sample” relates to assessing the ability of a subject's Giα and Gsα proteins to mediate signal transduction upon stimulation (e.g., with an appropriate GPCR ligand or agonist), and thus relate to the activity and not to the level of expression of Giα and Gsα proteins. In some embodiments, this can be done using a CellKey™ apparatus, as previously described (Akoume et al., 2010 and WO 2010/040234, 2010 to Moreau et al.).

The terms “suppressor”, “inhibitor” and “antagonist” are well known in the art and are used herein interchangeably. The expression inhibitors of PKA (e.g., PKA-γ2), inhibitors of PKC (e.g., PKC-η or PKC-ε), inhibitors of CaMK1 (e.g., CaMK1δ), inhibitors of CaMK4, inhibitors of CK (e.g., CK2) and inhibitors of CaMK2 include any compound able to negatively affect the activity of PKA (e.g., PKA-γ2), PKC (e.g., PKC-η or PKC-ε), CaMK1 (e.g., CaMK1δ), CaMK4, CK (e.g., CK2) and CaMK2, respectively by reducing for example its expression (i.e., at the transcriptional and/or translational level), the level of PKA (e.g., PKA-γ2), PKC (e.g., PKC-η or PKC-ε), CaMK1 (e.g., CaMK1δ), CaMK4, CK (e.g., CK2) and CaMK2 mRNA, respectively and/or protein, or an activity associated with PKA (e.g., PKA-γ2), PKC (e.g., PKC-η or PKC-ε), CaMK1 (e.g., CaMK1δ), CaMK4, CK (e.g., CK2) and CaMK2, respectively. It includes intracellular as well as extracellular suppressors. Without being so limited, such suppressors include RNA interference agents (siRNA, shRNA, miRNA), antisense molecules, ribozymes, proteins (e.g., dominant negative, inactive variants), peptides, small molecules, antibodies, antibody fragments, etc. Without being limited, inhibitors of PKA (e.g., PKA-γ2) include H89 cGMP dependent kinase inhibitor peptide; KT 5720; PKA inhibitor fragment (6-22) amide; PKI 14-22 amide, myristoylated. Without being limited, inhibitors of PKC (e.g., PKC-ε and PKC-η) include Gö6983, Gö6976; GF109203X; Dihydrosyphingosine; CID2858522; Chelerythrine chloride; CGP53353; Calphostin C; C-1; and Binsindolylmaleimide II. Without being limited, inhibitors of CaMK1 and CaMK4 (e.g., CaMK1δ) include STO609; NH 125; ML 9 hydrochloride; autocamtide-2-related inhibitory peptide; and arcyriaflavin A. Without being limited, inhibitors of CaMK2 include KN93, NH 125; ML 9 hydrochloride; autocamtide-2-related inhibitory peptide; and arcyriaflavin A. Without being limited, inhibitors of CK (e.g., CK1, CK2) include (R)-DRF053 dihydrochloride inhibits CK1), Ellagic acid (Selective inhibitor of CK2), LH 846 (Selective casein kinase 1δ inhibitor), PF 4800567 hydrochloride (Selective casein kinase 1ε inhibitor), PF 670462 (Potent and selective CK1ϵ and CK1δ inhibitor), TBB (Selective cell-permeable CK2 inhibitor), TMCB (inhibits CK2) and D4476 (Selective CK1 inhibitor).

In an embodiment, the PKA (e.g., PKA-γ2), PKC (e.g., PKC-η or PKC-ε), CaMK1 (e.g., CaMK1δ), CaMK4, CK (e.g., CK2) or CaMK2 inhibitor is a neutralizing antibody directed against (or specifically binding to) a human PKA (e.g., PKA-γ2), PKC (e.g., PKC-η or PKC-ε), CaMK1 (e.g., CaMK1δ), CaMK4, CK (e.g., CK2) and CaMK2 polypeptide, respectively. Antibodies are further described below.

The present invention also relates to methods for the determination of the level of expression (i.e., transcript (RNA) or translation product (protein)) of Gαi1 (having phosphorylated and/or unphosphorylated serine residues), Gαi2 (e.g., having phosphorylated and/or unphosphorylated serine residues), Gαi3 (e.g., having phosphorylated and/or unphosphorylated serine residues), and Gas. In specific embodiments, it also includes a method that comprises the determination of the level of expression of one or more other scoliosis markers. For example, it may include the determination of the level of expression (i.e., transcript or translation product) of OPN, sCD44, etc. as disclosed in co-pending WO 2008/119170 to Moreau. The present invention therefore encompasses any known method for such determination including Elisa (Enzyme Linked Immunosorbent Assay), RIA (Radioimmunoassay), real time PCR and competitive PCR, Northern blots, nuclease protection, plaque hybridization and slot blots.

### Antibodies

As used herein, the terms anti-PKA (e.g., PKA-γ2), PKC (e.g., PKC-η or PKC-ε), CaMK1 (e.g., CaMK1δ), CaMK4, CK (e.g., CK2), CaMK2, Giα1 (e.g., having phosphorylated and/or unphosphorylated serine residues), Giα2 (e.g., having phosphorylated and/or unphosphorylated serine residues), Giα3 (e.g., having phosphorylated and/or unphosphorylated serine residues) or Gsα antibody or “immunologically specific anti-PKA-γ2, PKC-ε, PKC-η, CaMK1δ, Giα1 (e.g., having phosphorylated and/or unphosphorylated serine residues), Giα2 (e.g., having phosphorylated and/or unphosphorylated serine residues), Giα3 (e.g., having phosphorylated and/or unphosphorylated serine residues), or Gsα antibody, refers to an antibody that specifically binds to (interacts with) a PKA (e.g., PKA-γ2), PKC (e.g., PKC-η or PKC-ε), CaMK1 (e.g., CaMK1δ), CaMK4, CK (e.g., CK2), CaMK2, Giα1 (e.g., having phosphorylated and/or unphosphorylated serine residues), Giα2 (e.g., having phosphorylated and/or unphosphorylated serine residues), Giα3 (e.g., having phosphorylated and/or unphosphorylated serine residues), or Gsα protein, respectively and displays no substantial binding to other naturally occurring proteins other than the ones sharing the same antigenic determinants as the PKA (e.g., PKA-γ2), PKC (e.g., PKC-η or PKC-ε), CaMK1 (e.g., CaMK1δ), CaMK4, CK (e.g., CK2), CaMK2, Giα1 (e.g., having phosphorylated and/or unphosphorylated serine residues), Giα2 (e.g., having phosphorylated and/or unphosphorylated serine residues), Giα3 (e.g., having phosphorylated and/or unphosphorylated serine residues), or Gsα protein. The term “antibody” or “immunoglobulin” is used in the broadest sense, and covers monoclonal antibodies (including full length monoclonal antibodies), polyclonal antibodies, multispecific antibodies, and antibody fragments so long as they exhibit the desired biological activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, VH regions (V_{H}, V_{H}-V_{H}), anticalins, PepBodies™, antibody-T-cell epitope fusions (Troybodies) or Peptibodies. Additionally, any secondary antibodies, either monoclonal or polyclonal, directed to the first antibodies would also be included within the scope of this invention. In an embodiment, the antibody is a monoclonal antibody. In another embodiment, the antibody is a humanized or CDR-grafted antibody.

Antibodies directed to PKA (e.g., PKA-γ2), PKC (e.g., PKC-η or PKC-ε), CaMK1 (e.g., CaMK1δ), CaMK4, CK (e.g., CK2), CaMK2, Giα1 (e.g., having phosphorylated and/or unphosphorylated serine residues), Giα2 (e.g., having phosphorylated and/or unphosphorylated serine residues), Giα3 (e.g., having phosphorylated and/or unphosphorylated serine residues) and Gsα are included within the scope of this invention as they can be produced by well established procedures known to those of skill in the art. Additionally, any secondary antibodies, either monoclonal or polyclonal, directed to the first antibodies would also be included within the scope of this invention.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories). The term antibody encompasses herein polyclonal, monoclonal antibodies and antibody variants such as single-chain antibodies, humanized antibodies, chimeric antibodies and immunologically active fragments of antibodies (e.g. Fab and Fab′ fragments) which inhibit or neutralize their respective interaction domains in Hyphen and/or are specific thereto.

Polyclonal antibodies are preferably raised in animals by multiple subcutaneous (sc), intravenous (iv) or intraperitoneal (ip) injections of the relevant antigen with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen, immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293.

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

As used herein, the term “purified” in the expression “purified antibody” is simply meant to distinguish man-made antibody from an antibody that may naturally be produced by an animal against its own antigens. Hence, raw serum and hybridoma culture medium containing anti-OPN antibody are “purified antibodies” within the meaning of the present invention.

The present invention also concerns isolated nucleic acid molecules including probes and primers to detect PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα (and optionally other scoliosis markers (e.g., OPN, sCD44, etc.). In specific embodiments, the isolated nucleic acid molecules have no more than 300, or no more than 200, or no more than 100, or no more than 90, or no more than 80, or no more than 70, or no more than 60, or no more than 50, or no more than 40 or no more than 30 nucleotides. In specific embodiments, the isolated nucleic acid molecules have at least 17, or at least 18, or at least 19, or at least 20, or at least 30, or at least 40 nucleotides. In other specific embodiments, the isolated nucleic acid molecules have at least 20 and no more than 300 nucleotides. In other specific embodiments, the isolated nucleic acid molecules have at least 20 and no more than 200 nucleotides. In other specific embodiments, the isolated nucleic acid molecules have at least 20 and no more than 100 nucleotides. In other specific embodiments, the isolated nucleic acid molecules have at least 20 and no more than 90 nucleotides. In other specific embodiments, the isolated nucleic acid molecules have at least 20 and no more than 80 nucleotides. In other specific embodiments, the isolated nucleic acid molecules have at least 20 and no more than 70 nucleotides. In other specific embodiments, the isolated nucleic acid molecules have at least 20 and no more than 60 nucleotides. In other specific embodiments, the isolated nucleic acid molecules have at least 20 and no more than 50 nucleotides. In other specific embodiments, the isolated nucleic acid molecules have at least 20 and no more than 40 nucleotides. In other specific embodiments, the isolated nucleic acid molecules have at least 17 and no more than 40 nucleotides. In other specific embodiments, the isolated nucleic acid molecules have at least 20 and no more than 30 nucleotides. In other specific embodiments, the isolated nucleic acid molecules have at least 17 and no more than 30 nucleotides. In other specific embodiments, the isolated nucleic acid molecules have at least 30 and no more than 300 nucleotides. In other specific embodiments, the isolated nucleic acid molecules have at least 30 and no more than 200 nucleotides. In other specific embodiments, the isolated nucleic acid molecules have at least 30 and no more than 100 nucleotides. In other specific embodiments, the isolated nucleic acid molecules have at least 30 and no more than 90 nucleotides. In other specific embodiments, the isolated nucleic acid molecules have at least 30 and no more than 80 nucleotides. In other specific embodiments, the isolated nucleic acid molecules have at least 30 and no more than 70 nucleotides. In other specific embodiments, the isolated nucleic acid molecules have at least 30 and no more than 60 nucleotides. In other specific embodiments, the isolated nucleic acid molecules have at least 30 and no more than 50 nucleotides. In other specific embodiments, the isolated nucleic acid molecules have at least 30 and no more than 40 nucleotides. It should be understood that in real-time PCR, primers also constitute probe without the traditional meaning of this term. Primers or probes appropriate to detect PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα in the methods of the present invention can be designed with known methods using sequences distributed across their respective nucleotide sequence.

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally known. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

As used herein the terms “detectably labeled” refer to a marking of a probe or an antibody in accordance with the presence invention that will allow the detection of PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα (and optionally other scoliosis markers (e.g., OPN, sCD44, etc.). in accordance with the present invention. Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods. Non-limiting examples of labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma 32P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

The present invention also relates to methods of selecting compounds. As used herein the term “compound” is meant to encompass natural, synthetic or semi-synthetic compounds, including without being so limited chemicals, macromolecules, cell or tissue extracts (from plants or animals), nucleic acid molecules, peptides, antibodies and proteins.

The present invention also relates to arrays. As used herein, an “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

As used herein “array of nucleic acid molecules” is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus, the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

As used herein “solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Any known nucleic acid arrays can be used in accordance with the present invention. For instance, such arrays include those based on short or longer oligonucleotide probes as well as cDNAs or polymerase chain reaction (PCR) products. Other methods include serial analysis of gene expression (SAGE), differential display, as well as subtractive hybridization methods, differential screening (DS), RNA arbitrarily primer (RAP)-PCR, restriction endonucleolytic analysis of differentially expressed sequences (READS), amplified restriction fragment-length polymorphisms (AFLP).

The present invention encompasses using antibodies for detecting or determining Gαi1 (e.g., having phosphorylated and/or unphosphorylated serine residues), Gαi2 (having e.g., having phosphorylated and/or unphosphorylated serine residues serine residues), Gαi3 (e.g., having phosphorylated and/or unphosphorylated serine residues), Gas levels for instance in the samples of a subject and for including in kits of the present invention. Antibodies that specifically bind to these biological markers can be produced routinely with methods further described above. The present invention also encompasses using antibodies commercially available. Without being so limited antibodies that specifically bind to Gαi1 (e.g., having phosphorylated and/or unphosphorylated serine residues), Gαi2 (e.g., having phosphorylated and/or unphosphorylated serine residues), Gαi3 (e.g., having phosphorylated and/or unphosphorylated serine residues), Gas include those listed in Table I below.

TABLE I
Commercially available antibodies for Gαi1 (e.g., having phosphorylated and/or
unphosphorylated serine residues), Gαi2 (e.g., having phosphorylated and/or
unphosphorylated serine residues), Gαi3 (e.g., having phosphorylated and/or
unphosphorylated serine residues), and Gαs.

| Description | Supplier | Catalogue Number | Reactivity | Application | Host |
|---|---|---|---|---|---|
| G protein alpha inhibitor 1 | Abcam | ab136510 | Human, Rat | WB | Mouse |
| antibody [7H7] | | | | | |
| G protein alpha inhibitor 1 | Abcam | ab140333 | Human, Mouse, Rat, | WB | Mouse |
| antibody [R4.5] | | | Cow, Guinea pig | | |
| G protein alpha inhibitor 1 | Abcam | ab102014 | Human | WB | Rabbit |
| antibody | | | | | |
| G protein alpha inhibitor 1 | Abcam | ab55103 | Human | IHC-P, WB | Mouse |
| antibody | | | | | |
| G protein alpha inhibitor 1 | Abcam | ab81447 | Human | ELISA, WB | Rabbit |
| antibody | | | | | |
| G protein alpha inhibitor 1 | Abcam | ab19932 | Human | WB | Mouse |
| antibody [SPM397] | | | | | |
| G protein alpha inhibitor 1 | Abcam | ab118434 | Human | ELISA, IHC-P, WB | Mouse |
| antibody [2B8-2A5] | | | | | |
| G protein alpha inhibitor 1 | Abcam | ab140125 | Human, Mouse, Rat | Flow Cyt, ICC, IHC- | Rabbit |
| antibody [EPR9441(B)] | | | | P, WB | |
| G protein alpha Inhibitor 2 | Abcam | ab118578 | Human | IHC-P, WB | Rabbit |
| antibody | | | | | |
| G protein alpha Inhibitor 2 | Abcam | ab55117 | Human | WB | Mouse |
| antibody | | | | | |
| G protein alpha Inhibitor 2 | Abcam | ab81452 | Human | ELISA, WB | Rabbit |
| antibody | | | | | |
| G protein alpha Inhibitor 2 | Abcam | ab123427 | Human | WB | Rabbit |
| antibody | | | | | |
| G Protein alpha Inhibitor | Abcam | ab3522 | Human, Mouse, Rat | IHC-P, WB | Rabbit |
| 1 + 2 antibody | | | | | |
| G protein alpha Inhibitor 2 | Abcam | ab154155 | Human | ICC/IF, WB | Rabbit |
| antibody | | | | | |
| G protein alpha Inhibitor 2 | Abcam | ab157204 | Human, Mouse, Rat | Flow Cyt, ICC/IF, | Rabbit |
| antibody [EPR9469] | | | | IHC-P, WB | |
| G protein alpha Inhibitor 2 | Abcam | ab137050 | Human, Mouse, Rat | Flow Cyt, WB | Rabbit |
| antibody [EPR9468] | | | | | |
| G protein alpha Inhibitor 2 | Abcam | ab20392 | Human, Rat | ICC/IF, IP, WB | Rabbit |
| antibody | | | | | |
| G protein alpha Inhibitor 2 | Abcam | ab78193 | Human, Mouse, Rat, | WB | Mouse |
| antibody [L5] | | | Cow, Guinea pig | | |
| GNAI1 Antibody | Abgent | AT2225a | Human | WB, IHC, E | Mouse |
| (monoclonal) (M01) | | | | | |
| GNAI1 polyclonal antibody | Abnova | H00002770- | Human | ELISA, WB | Mouse |
| (A01) | CorpoRation | A01 | | | |
| GNAI1 monoclonal | Abnova | H00002770- | Human | WB-Tr, WB-Ti, S- | Mouse |
| antibody (M01), clone 2B8- | CorpoRation | M01 | | ELISA, ELISA, WB- | |
| 2A5 | | | | Re, IHC-P | |
| anti G protein alpha | Acris | AM05302PU-N | Cow, Guinea Pig, | WB | Mouse |
| inhibitor 1 | Antibodies | | Human, Mouse, Rat | | |
| | GmbH | | | | |
| anti G protein alpha | Acris | SP5158P | Human, Mouse, Rat | WB | Rabbit |
| inhibitor 1 (+ alpha 2) | Antibodies | | | | |
| | GmbH | | | | |
| anti G protein alpha | Acris | AM12136PU-N | Cow, Guinea pig, | P, WB | Mouse |
| inhibitor 1 | Antibodies | | Human, Mouse, Pig, | | |
| | GmbH | | Rat, Rat | | |
| anti G protein alpha | Acris | AM20888PU-N | Human | E, P, WB | Mouse |
| inhibitor 1 | Antibodies | | | | |
| | GmbH | | | | |
| anti G protein alpha | Acris | AP05163PU-N | Cow, Human, Mouse, | WB | Rabbit |
| inhibitor 1 (+ GNAI2) | Antibodies | | Rat | | |
| | GmbH | | | | |
| anti G protein alpha | Acris | AP09041PU-N | Cow, Human, Rat, | P, WB | Rabbit |
| inhibitor 1 (+ GNAI2) | Antibodies | | Mouse | | |
| | GmbH | | | | |
| anti G protein alpha | Acris | AM32641SU-N | Cow, Guinea pig, | WB | Mouse |
| inhibitor 1 | Antibodies | | Human, Mouse, Pig, | | |
| | GmbH | | Rat, Rat | | |
| anti G protein alpha | Acris | AM32537PU-N | VeRatebRates | WB | Mouse |
| inhibitor 1 (110-123) | Antibodies | | | | |
| | GmbH | | | | |
| anti-G Protein Alpha | antibodies- | ABIN609015 | Human | WB, IHC | Rabbit |
| Inhibitor 1/2 | online | | | | |
| (GNAI1/GNAI2) (AA 345-354) | | | | | |
| antibody | | | | | |
| anti-Guanine Nucleotide | antibodies- | ABIN406409 | Cow | WB | Rabbit |
| Binding Protein (G Protein), | online | | (Cow), Human, Mouse | | |
| alpha Inhibiting Activity | | | (Murine), Rat | | |
| Polypeptide 1 (GNAI1) | | | (Rattus), Fruit Fly | | |
| antibody | | | (Drosophila | | |
| | | | melanogaster), Pig | | |
| | | | (Porcine), Xenopus | | |
| | | | laevis, Chicken | | |
| anti-Guanine Nucleotide | antibodies- | ABIN393287 | Human | ELISA, WB, IHC | Mouse |
| Binding Protein (G Protein), | online | | | | |
| alpha Inhibiting Activity | | | | | |
| Polypeptide 1 (GNAI1) | | | | | |
| antibody | | | | | |
| anti-Guanine Nucleotide | antibodies- | ABIN203567 | Human | ELISA | Rabbit |
| Binding Protein (G Protein), | online | | | | |
| alpha Inhibiting Activity | | | | | |
| Polypeptide 1 (GNAI1) (AA | | | | | |
| 345-354) antibody | | | | | |
| anti-Guanine Nucleotide | antibodies- | ABIN574258 | Human | ELISA, WB, IHC | Mouse |
| Binding Protein (G Protein), | online | | | | |
| alpha Inhibiting Activity | | | | | |
| Polypeptide 1 (GNAI1) | | | | | |
| antibody | | | | | |
| anti-Guanine Nucleotide | antibodies- | ABIN211172 | Human | WB | Mouse |
| Binding Protein (G Protein), | online | | | | |
| alpha Inhibiting Activity | | | | | |
| Polypeptide 1 (GNAI1) (AA | | | | | |
| 110-123) antibody | | | | | |
| anti-Guanine Nucleotide | antibodies- | ABIN470853 | Human | WB, IP | Rabbit |
| Binding Protein (G Protein), | online | | | | |
| alpha Inhibiting Activity | | | | | |
| Polypeptide 3 (GNAI3) (C- | | | | | |
| Term) antibody | | | | | |
| anti-Guanine Nucleotide | antibodies- | ABIN1010195 | Human, Rat, Mouse, Cow, | ELISA, WB, IHC | Rabbit |
| Binding Protein (G Protein), | online | | Cat, Dog, Chicken | | |
| alpha Inhibiting Activity | | | | | |
| Polypeptide 1 (GNAI1) | | | | | |
| antibody | | | | | |
| anti-Guanine Nucleotide | antibodies- | ABIN322115 | Human | WB, ELISA | Rabbit |
| Binding Protein (G Protein), | online | | | | |
| alpha Inhibiting Activity | | | | | |
| Polypeptide 1 (GNAI1) | | | | | |
| (Internal Region) antibody | | | | | |
| anti-G Protein Alpha | antibodies- | ABIN472931 | Human | WB | Rabbit |
| Inhibitor 1/2 | online | | | | |
| (GNAI1/GNAI2) (AA 346-355) | | | | | |
| antibody | | | | | |
| GNAI1 antibody - middle | Aviva | ARP54630_P050 | Human, Mouse, Rat, | WB | Rabbit |
| region (ARP54630_P050) | SysteMouse | | Drosophila, Pig, Cow, | | |
| | Biology | | Fruit fly, African clawed | | |
| | | | frog, Chicken | | |
| Rabbit Anti-G Protein alpha | Bioss Inc. | bs-8556R | Human, Mouse, Rat, | WB, ELISA, IHC-P, | Rabbit |
| Inhibitor 1 Polyclonal | | | Cow, Dog | IHC-F | |
| Antibody | | | | | |
| Rabbit Anti-G protein alpha | Bioss Inc. | bs-9920R | Human, Mouse, Rat, | WB, ELISA, IHC- | Rabbit |
| Inhibitor 2 Polyclonal | | | Dog, Pig, Chicken, | P, IHC-F | |
| Antibody | | | Cow, Sheep | | |
| Gα(i) Antibody | Cell | 5290S | Human, Mouse, Rat | WB | Rabbit |
| | Signaling | | | | |
| | Technology | | | | |
| Anti-G Protein Gialpha-1, | EMD | MAB3075 | Cow, Guinea pig, | WB | |
| clone R4 | Millipore | | Human, Mouse, Pig, | | |
| | | | Rat, Rat | | |
| Anti-Gialpha-2-Subunit, | EMD | 371727-50UL | Human, Mouse, Rat | ELISA, WB | Rabbit |
| Internal (85-100) Rabbit | Millipore | | | | |
| pAb | | | | | |
| Anti-G Protein Gialpha-2, | EMD | MAB3077 | Cow, Guinea pig, | WB | Mouse |
| clone L5 | Millipore | | Human, Mouse, Pig, | | |
| | | | Rat, Rat | | |
| G-Protein Subunit Antibody | EMD | 371770-1ST | N/A | WB | Rabbit |
| Set | Millipore | | | | |
| GNAI1 antibody | Fitzgerald | 70R-2047 | Human, Mouse, Rat, Drosophila | WB | Rabbit |
| | Industries | | | | |
| | International | | | | |
| Chicken Anti-Gai3 (G | GenWay | 15-288- | Human, Mouse, Rat | ICC, WB | Chicken |
| protein Gi alpha subunit 3) | Biotech, Inc. | 22489 | | | |
| GNAI1 antibody - middle | GenWay | GWB- | Human Mouse Rat | WB | Rabbit |
| region | Biotech, Inc. | MT472D | Drosophila Pig Cow | | |
| | | | Fruit fly African clawed | | |
| | | | frog Chicken | | |
| Anti-Gi/GNAI1 | LifeSpan | LS-B4333-50 | Human | ELISA, IHC-P, WB | Mouse |
| | BioSciences | | | | |
| Anti-Gi/GNAI1 | LifeSpan | LS-C26697- | Cow, Human, Rat | WB | Mouse |
| | BioSciences | 100 | | | |
| Anti-Gi/GNAI1 | LifeSpan | LS-C87973- | Cow, Guinea pig, | WB | Rat |
| | BioSciences | 200 | Human, Mouse, Pig, | | |
| | | | Rat, Rat | | |
| Anti-Gi/GNAI1 | LifeSpan | LS-B2546-50 | Rat, Cow, Guinea pig, | IHC-P, WB | Mouse |
| | BioSciences | | Human, Mouse, Rat | | |
| Anti-Gi/GNAI1 | LifeSpan | LS-C3237- | Human | ELISA | Rabbit |
| | BioSciences | 100 | | | |
| Anti-Gi/GNAI2 | LifeSpan | LS-C87972- | Cow, Guinea pig, | WB | |
| | BioSciences | 100 | Human, Mouse, Pig, | | |
| | | | Rat, Rat | | |
| Anti-Gi/GNAI3 | LifeSpan | LS-C23979- | Cow, Guinea pig, | WB | Mouse |
| | BioSciences | 100 | Mouse, Rat, Cow | | |
| Anti-Gi/GNAI1 | LifeSpan | LS-C81891- | Cow, Chicken, | WB | |
| | BioSciences | 50 | Drosophila, Xenopus, | | |
| | | | Human, Mouse, Pig, | | |
| | | | Rat, Human | | |
| Anti-Gi/GNAI1 | LifeSpan | LS-C23978- | Human | ELISA, WB | Mouse |
| | BioSciences | 200 | | | |
| Anti-G Protein Alpha | LifeSpan | LS-C121228- | Human, Cow, Human, | IHC-P, WB | Rabbit |
| Inhibitor 1/2 | BioSciences | 100 | Mouse, Rat | | |
| (GNAI1/GNAI2) | | | | | |
| Anti-GNAI3 | LifeSpan | LS-C23813- | Hamster, Human, | IP, WB | Rabbit |
| | BioSciences | 50 | Mouse, Rat | | |
| Anti-G Protein Gialpha-1, | Merck | MAB3075 | Cow, Guinea pig, | WB | |
| clone R4 | Millipore | | Human, Mouse, Pig, | | |
| | | | Rat, Rat | | |
| Anti-G Protein Gialpha 1/2 | Merck | 07-1500 | Human, Mouse, Rat, | WB, IH(P) | |
| | Millipore | | Cow | | |
| Anti-Gialpha-2-Subunit, | Merck | 371727-50UL | Human, Mouse, Rat | ELISA, WB | Rabbit |
| Internal (85-100) Rabbit | Millipore | | | | |
| pAb | | | | | |
| Rabbit anti-Human guanine | MyBioSource.com | MBS716824 | Human, Mouse, Rat. | ELISA, WB, IHC | Rabbit |
| nucleotide binding protein | | | | | |
| (G protein), alpha inhibiting | | | | | |
| activity polypeptide 1 | | | | | |
| polyclonal Antibody | | | | | |
| G protein alpha inhibitor 1 | Novus | NBP1-52926 | Human, Mouse, Rat, | WB | Rabbit |
| Antibody | Biologicals | | Cow, Chicken, | | |
| | | | Drosophila, Porcine, | | |
| | | | Xenopus | | |
| G protein alpha inhibitor 1 | Novus | H00002770- | Human | WB, ELISA | Mouse |
| Antibody | Biologicals | A01 | | | |
| G protein alpha inhibitor 1 | Novus | NBP1-31601 | Human | WB | Rabbit |
| Antibody | Biologicals | | | | |
| G protein alpha inhibitor 1 | Novus | H00002770- | Human | WB, ELISA, IHC | Mouse |
| Antibody (2B8-2A5) | Biologicals | M01 | | | |
| G protein alpha inhibitor 1 | Novus | NBP2-16558 | Human | WB, IHC | Rabbit |
| Antibody | Biologicals | | | | |
| G protein alpha inhibitor 1 | Novus | NBP1-40247 | Human, Mouse, Rat, | WB, IHC | Mouse |
| Antibody (R4.5) | Biologicals | | Cow, Guinea Pig | | |
| G protein alpha inhibitor | Novus | NB120-3522 | Human, Mouse, Rat | WB, IP | Rabbit |
| 1/2 Antibody | Biologicals | | | | |
| GNAI1 | Proteintech | 12617-1-AP | Human, Mouse, Rat | ELISA, WB, IHC | Rabbit |
| | Group Inc | | | | |
| Galpha i-1 (SPM397) | Santa Cruz | sc-56536 | Mouse, Rat, Human, | WB, IP | Mouse |
| | Biotechnology, | | cow | | |
| | Inc. | | | | |
| Galpha i-1 (R4) | Santa Cruz | sc-13533 | Mouse, Rat, Human, | WB, IP, IF, IHC(P) | Mouse |
| | Biotechnology, | | cow | | |
| | Inc. | | | | |
| Galpha i-1/3 (I-18) | Santa Cruz | sc-26762 | Mouse, Rat, Human | WB, IF, ELISA | Goat |
| | Biotechnology, | | | | |
| | Inc. | | | | |
| Galpha i-1 (I-20) | Santa Cruz | sc-391 | Mouse, Rat, Human | WB, IF, ELISA | Rabbit |
| | Biotechnology, | | | | |
| | Inc. | | | | |
| Galpha i-3 (C-10) | Santa Cruz | sc-262 | Mouse, Rat, Human | WB, IP, IF, ELISA | Rabbit |
| | Biotechnology, | | | | |
| | Inc. | | | | |
| Galpha i-3 (H-7) | Santa Cruz | sc-365422 | Mouse, Rat, Human | WB, IP, IF, ELISA | Mouse |
| | Biotechnology, | | | | |
| | Inc. | | | | |
| Galpha i-1/2/3 (N-20) | Santa Cruz | sc-26761 | Mouse, Rat, Human | WB, IP, IF, ELISA | Goat |
| | Biotechnology, | | | | |
| | Inc. | | | | |
| Galpha i/o/t/z/gust (H-300) | Santa Cruz | sc-28586 | Mouse, Rat, Human | WB, IP, IF, ELISA | Rabbit |
| | Biotechnology, | | | | |
| | Inc. | | | | |
| Galpha i/o/t/z (D-15) | Santa Cruz | sc-12798 | Mouse, Rat, Human | WB, IP, IF, ELISA | Goat |
| | Biotechnology, | | | | |
| | Inc. | | | | |
| G protein alpha Inhibitor 1 | Thermo | PA5-30043 | Human | IHC (P), WB | Rabbit |
| Antibody | Scientific | | | | |
| | Pierce | | | | |
| | Antibodies | | | | |
| G protein alpha inhibitor 1 | Thermo | PA5-28223 | Human | WB | Rabbit |
| Antibody | Scientific | | | | |
| | Pierce | | | | |
| | Antibodies | | | | |
| Gia-1 Antibody | Thermo | MA1-12610 | Cow, Guinea pig, | WB | Mouse |
| | Scientific | | Human, Mouse, Pig, | | |
| | Pierce | | Rat, Rat | | |
| | Antibodies | | | | |
| Galphai1 G-Protein | Thermo | MA5-12800 | Cow, Guinea pig, | WB | Mouse |
| Antibody | Scientific | | Human, Mouse, Pig, | | |
| | Pierce | | Rat, Rat | | |
| | Antibodies | | | | |
| G protein alpha Inhibitor 2 | Thermo | PA5-27496 | Human, Mouse | WB | Rabbit |
| Antibody | Scientific | | | | |
| | Pierce | | | | |
| | Antibodies | | | | |
| G protein alpha Inhibitor 2 | Thermo | PA5-27520 | Human | IF, WB | Rabbit |
| Antibody | Scientific | | | | |
| | Pierce | | | | |
| | Antibodies | | | | |
| G protein alpha S antibody | Abcam | ab97629 | Human | WB | Rabbit |
| G protein alpha S antibody | Abcam | ab83735 | Human, Mouse | IHC-P, WB | Rabbit |
| G protein alpha S antibody | Abcam | ab101736 | Human, Mouse, Rat | WB | Goat |
| G protein alpha S antibody | Abcam | ab97663 | Human, Mouse | IHC-P, WB | Rabbit |
| GNAS Antibody (Center) | Abgent | AP6865c | Human | WB, ELISA | Rabbit |
| GNAS Antibody (C-term) | Abgent | AP13065b | Human | WB, IHC, E | Rabbit |
| GNAS purified MaxPab | Abnova | H00002778- | Human | PLA-Ce, Det Ab, WB- | Rabbit |
| rabbit polyclonal antibody | Corporation | D01P | | Tr | |
| (D01P) | | | | | |
| GNAS monoclonal | Abnova | H00002778-M | Human | WB, ELISA | Mouse |
| antibody | Corporation | | | | |
| GNAS polyclonal antibody | Abnova | PAB18557 | Human | ELISA, WB-Ce | Goat |
| | Corporation | | | | |
| anti GNAS/GSP | Acris | AP20123PU-N | Human, Cow, Mouse, | WB, ELISA | Goat |
| | Antibodies | | Rat | | |
| | GmbH | | | | |
| anti-Galpha S (AA 11-21) | antibodies- | ABIN968909 | Human | WB | Mouse |
| antibody | online | | | | |
| anti-GNAS Complex Locus | antibodies- | ABIN571171 | Human | ELISA, WB | Goat |
| (GNAS) antibody | online | | | | |
| anti-GNAS Complex Locus | antibodies- | ABIN1086769 | Human | WB, ELISA | Rabbit |
| (GNAS) antibody | online | | | | |
| anti-GNAS Complex Locus | antibodies- | ABIN604875 | Human | WB, IHC | Rabbit |
| (GNAS) antibody | online | | | | |
| anti-GNAS Complex Locus | antibodies- | ABIN1035879 | Human, Rat, Mouse | WB, IHC, ELISA | Rabbit |
| (GNAS) (Transcript Variant | online | | | | |
| 1) antibody | | | | | |
| anti-GNAS Complex Locus | antibodies- | ABIN213649 | Human | ELISA, IH | Rabbit |
| (GNAS) (AA 385-394) | online | | | | |
| antibody | | | | | |
| anti-GNAS Complex Locus | antibodies- | ABIN656989 | Human | WB, ELISA | Rabbit |
| (GNAS) (C-Term) antibody | online | | | | |
| anti-GNAS Complex Locus | antibodies- | ABIN203445 | Human | IH, WB | Rabbit |
| (GNAS) (N-Term) antibody | online | | | | |
| GNAS antibody-N- | Aviva | ARP42693_P050 | Human, Mouse, Rat, | WB, IHC | Rabbit |
| terminal region | Systems | | Dog, Bovine, Pig | | |
| (ARP42693_P050) | Biology | | | | |
| GNAS antibody-N- | Aviva | ARP41764_T100 | Human, Dog, Mouse, | WB | Rabbit |
| terminal region | Systems | | Rat, Pig, Rabbit, Cow | | |
| (ARP41764_T100) | Biology | | | | |
| Anti-GNAS | Biomatik | CAE21174 | Human | Pep-ELISA, WB | Goat |
| G protein alpha S antibody | Biorbyt | orb6103 | Human, Mouse, Rat | P-ELISA, WB, IHC-P | Rabbit |
| GNAS antibody | Biorbyt | orb31105 | Human | WB, ELISA | Rabbit |
| GNAS antibody | Biorbyt | orb37122 | Human | WB, ELISA | Rabbit |
| GNAS antibody | Biorbyt | orb20551 | Human, Mouse, Rat, | ELISA, WB | Goat |
| | | | Cow | | |
| Rabbit Anti-G protein alpha | Bioss Inc. | bs-3939R | Human, Mouse, Rat, | WB, ELISA, IP, IHC- | Rabbit |
| S Polyclonal Antibody | | | Dog, Pig, Horse, | P, IHC-F, IF | |
| | | | Chicken, Cow, Rabbit | | |
| Anti-Gsalpha | EMD | 06-237 | Cow, Human, Mouse | IC, IP, WB | Rabbit |
| | Millipore | | | | |
| GNAS antibody | Fitzgerald | 70R-12940 | Human, Mouse | IHC-P, WB | Rabbit |
| | Industries | | | | |
| | International | | | | |
| GNAS antibody | Fitzgerald | 70R-1651 | Human, Mouse, Rat, Dog | WB | Rabbit |
| | Industries | | | | |
| | International | | | | |
| GNAS antibody | Fitzgerald | 70R-1652 | Human | WB, IHC | Rabbit |
| | Industries | | | | |
| | International | | | | |
| GNAS antibody | Fitzgerald | 70R-5756 | Human, Mouse, Rat, Dog | WB | Rabbit |
| | Industries | | | | |
| | International | | | | |
| GNAS antibody | Fitzgerald | 70R-1643 | Human, Dog | WB | Rabbit |
| | Industries | | | | |
| | International | | | | |
| GNAS antibody [C1C3] | GeneTex | GTX113338 | NA | WB-Ag | Rabbit |
| Rabbit Anti-Human GNAS | GenWay | 18-003- | Human | Immunohistochemistry- | Rabbit |
| Polyclonal Antibody, | Biotech, Inc | 43749 | | P, Western Blot | |
| Unconjugated | | | | | |
| GNAS antibody-N- | GenWay | GWB- | Human, Mouse, Rat, | WB | Rabbit |
| terminal region | Biotech, Inc. | MP242H | Dog, Cow, Pig | | |
| GNAS antibody-N- | GenWay | GWB- | Human, Dog, Mouse, | WB | Rabbit |
| terminal region | Biotech, Inc. | MN943G | Rat, Pig, Rabbit, Cow | | |
| Rabbit Anti-GNAS | GenWay | 18-003- | Human, Mouse, Rat, | WB | Rabbit |
| Polyclonal Antibody, | Biotech, Inc. | 44268 | Dog | | |
| Unconjugated | | | | | |
| Anti-GNAS | LifeSpan | LS-B102-50 | Human, Primate, | ELISA, IHC-P | Rabbit |
| | BioSciences | | Human | | |
| Anti-GNAS | LifeSpan | LS-B4790-50 | Human, Primate, | ELISA, IHC-P | Rabbit |
| | BioSciences | | Human | | |
| Anti-GNAS | LifeSpan | LS-B4007-50 | Human, Bovine, Dog, | IHC-P, WB | Rabbit |
| | BioSciences | | Human, Mouse, Pig, | | |
| | | | Rat | | |
| Anti-GNAS | LifeSpan | LS-C31914- | Bovine, Human, | IHC, WB | Rabbit |
| | BioSciences | 100 | Mouse, Pig, Rabbit, | | |
| | | | Rat, Human | | |
| Anti-GNAS | LifeSpan | LS-C113002- | Human, Mouse, Rat, | ELISA, WB | Goat |
| | BioSciences | 100 | Human | | |
| Anti-GNAS | LifeSpan | LS-C80456- | Bovine, Dog, Human, | WB | Rabbit |
| | BioSciences | 100 | Mouse, Pig, Rabbit, | | |
| | | | Rat, Human | | |
| Anti-Gs protein, alpha | Merck | MABN543 | Human, Rat | WB | Mouse |
| subunit, clone N192/12 | Millipore | | | | |
| GNAS Antibody | ProSci, Inc | 42-594 | Human | ELISA, WB | Goat |
| GNAS Antibody | ProSci, Inc | 29-639 | Human, Dog | WB, ELISA | Rabbit |
| GNAS antibody | ProSci, Inc | 25-797 | Human, Mouse, Rat, Dog | ELISA, Western Blot | Rabbit |
| GNAS Antibody | ProSci, Inc | 29-791 | Human | ELISA, Western | Rabbit |
| | | | | Blot, | |
| | | | | Immunohistochemistry | |
| GNAS Antibody | ProSci, Inc | 29-640 | Human, Mouse, Rat, | ELISA, Western Blot | Rabbit |
| | | | Dog | | |
| Galpha s (C-19) | Santa Cruz | sc-46976 | Mouse, Rat, Human | WB, IF, ELISA | Goat |
| | Biotechnology, | | | | |
| | Inc. | | | | |
| Galpha s (12) | Santa Cruz | sc-135914 | Mouse, Rat, Human | WB, IP | Mouse |
| | Biotechnology, | | | | |
| | Inc. | | | | |
| Galpha 12 (S-20) | Santa Cruz | sc-409 | Mouse, Rat, Human | WB, IP, IF, IHC(P), | Rabbit |
| | Biotechnology, | | | ELISA | |
| | Inc. | | | | |
| Galpha s (C-17) | Santa Cruz | sc-46975 | Mouse, Rat, Human | WB, IP, IF, IHC(P), | Goat |
| | Biotechnology, | | | ELISA | |
| | Inc. | | | | |
| Galpha s (A-16) | Santa Cruz | sc-26766 | Mouse, Rat, Human | WB, IP, IF, ELISA | Goat |
| | Biotechnology, | | | | |
| | Inc. | | | | |
| Galpha s (K-20) | Santa Cruz | sc-823 | Mouse, Rat, Human | WB, IP, IF, IHC(P), | Rabbit |
| | Biotechnology, | | | ELISA | |
| | Inc. | | | | |
| Galpha s/olf (E-7) | Santa Cruz | sc-55546 | Mouse, Rat, Human | WB, IP, IF, IHC(P), | Mouse |
| | Biotechnology, | | | ELISA | |
| | Inc. | | | | |
| Galpha s/olf (A-5) | Santa Cruz | sc-55545 | Mouse, Rat, Human | WB, IP, IF, IHC(P), | Mouse |
| | Biotechnology, | | | ELISA | |
| | Inc. | | | | |
| Galpha s/olf (C-18) | Santa Cruz | sc-383 | Mouse, Rat, Human | WB, IP, IF, ELISA | Rabbit |
| | Biotechnology, | | | | |
| | Inc. | | | | |
| Galpha s/olf (G-10) | Santa Cruz | sc-365855 | Mouse, Rat, Human | WB, IP, IF, ELISA | Mouse |
| | Biotechnology, | | | | |
| | Inc. | | | | |
| Galpha s/olf (H-300) | Santa Cruz | sc-28585 | Mouse, Rat, Human | WB, IP, IF, ELISA | Rabbit |
| | Biotechnology, | | | | |
| | Inc. | | | | |
| Galpha s/olf (C-10) | Santa Cruz | sc-377435 | Mouse, Rat, Human | WB, IP, IF, ELISA | Mouse |
| | Biotechnology, | | | | |
| | Inc. | | | | |
| GNAS Antibody | Thermo | PA5-19315 | Human | WB | Goat |
| | Scientific | | | | |
| | Pierce | | | | |
| | Antibodies | | | | |

The present invention also encompasses arrays to detect and/or quantify the translation products of PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα. Such arrays include protein micro- or macroarrays, gel technologies including high-resolution 2D-gel methodologies, possibly coupled with mass spectrometry imaging system at the cellular level such as microscopy combined with a fluorescent labeling system.

The present invention also encompasses methods to screen/select for potential useful therapeutic agents using whole cells assays, the therapeutic compound being able to decrease the transcription and/or synthesis and/or activity (e.g., phosphorylation of Gi proteins) of PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα. Cells for use in such methods includes cells of any source (including in house or commercially available cell lines) and type (any tissue). In house cell lines could be made for instance by immortalizing cells from AIS subjects. In specific embodiments, methods of screening of the invention seek to identify agents that inhibit the PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα expression (transcription and/or translation). Useful cell lines for these embodiments include those producing high levels of PKA (e.g., PKA-γ2), PKC (e.g., PKC-η or PKC-ε), CaMK1 (e.g., CaMK1δ), CaMK4, CK (e.g., CK2), CaMK2, Giα1, Giα2, Giα3 or Gsα. They include osteoblasts, PMBcs and myoblasts.

In a particular embodiment, it includes cells of any cell type derived from a scoliotic patient (whole cell assay). In specific embodiments, it includes osteoblasts, chondrocytes, myoblasts or blood cells including PBMCs including lymphocytes. As used herein, the term “cell derived from a scoliotic patient” refers to cells isolated directly from scoliotic patients, or immortalized cell lines originating from cells isolated directly from scoliotic patients. In specific embodiments, the cells are paraspinal muscle cells. Such cells may be isolated by a subject through needle biopsies for instance.

Pharmaceutical compositions can also be administered by routes such as nasally, intravenously, intramuscularly, subcutaneously, sublingually, intrathecally, or intradermally. The route of administration can depend on a variety of factors, such as the environment and therapeutic goals.

### Dosage

Any amount of a pharmaceutical and/or nutraceutical and/or dietary supplement compositions can be administered to a subject. The dosages will depend on many factors including the mode of administration. Typically, the amount of anti-scoliosis composition (e.g., agent that decrease the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 contained within a single dose will be an amount that effectively prevents, delays or reduces scoliosis without inducing significant toxicity “therapeutically effective amount”.

The effective amount of the agent that agent that decrease the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 may also be measured directly. The effective amount may be given daily or weekly or fractions thereof. Typically, a pharmaceutical and/or nutraceutical and/or dietary supplement composition of the invention can be administered in an amount from about 0.001 mg up to about 500 mg per kg of body weight per day (e.g., 10 mg, 50 mg, 100 mg, or 250 mg). Dosages may be provided in either a single or multiple dosage regimen. For example, in some embodiments the effective amount is a dose that ranges from about 1 mg to about 25 grams of the anti-scoliosis preparation per day, about 50 mg to about 10 grams of the anti-scoliosis preparation per day, from about 100 mg to about 5 grams of the anti-scoliosis preparation per day, about 1 gram of the anti-scoliosis preparation per day, about 1 mg to about 25 grams of the anti-scoliosis preparation per week, about 50 mg to about 10 grams of the anti-scoliosis preparation per week, about 100 mg to about 5 grams of the anti-scoliosis preparation every other day, and about 1 gram of the anti-scoliosis preparation once a week.

By way of example, a pharmaceutical (e.g., containing an agent that decreases the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 composition of the invention can be in the form of a liquid, solution, suspension, pill, capsule, tablet, gelcap, powder, gel, ointment, cream, nebulae, mist, atomized vapor, aerosol, or phytosome. For oral administration, tablets or capsules can be prepared by conventional means with at least one pharmaceutically acceptable excipient such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Preparations for oral administration also can be suitably formulated to give controlled release of the active ingredients.

In addition, a pharmaceutical (e.g., containing an agent that decreases the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 composition of the invention can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration.

An agent that decreases the transcription and/or synthesis and/or stability and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 may be incorporated into dosage forms in conjunction with any of the vehicles which are commonly employed in pharmaceutical preparations, e.g. talc, gum arabic, lactose, starch, magnesium stearate, cocoa butter, aqueous or non-aqueous solvents, oils, paraffin derivatives or glycols. Emulsions such as those described in U.S. Pat. No. 5,434,183, may also be used in which vegetable oil (e.g., soybean oil or safflower oil), emulsifying agent (e.g., egg yolk phospholipid) and water are combined with glycerol. Methods for preparing appropriate formulations are well known in the art (see e.g., Remington's Pharmaceutical Sciences, 16th Ed., 1980, A. Oslo Ed., Easton, Pa.).

In cases where parenteral administration is elected as the route of administration, preparations containing an agent that decreases the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

These are simply guidelines since the actual dose must be carefully selected and titrated by the attending physician based upon clinical factors unique to each patient or by a nutritionist. The optimal daily dose will be determined by methods known in the art and will be influenced by factors such as the age of the patient and other clinically relevant factors. In addition, patients may be taking medications for other diseases or conditions. The other medications may be continued during the time that the agent that decreases the transcription and/or synthesis and/or activity (e.g., phosphorylation of Gi proteins) and/or stability of PKA (e.g., PKA-γ2), PKC (e.g., PKC-η or PKC-ε), CaMK1 (e.g., CaMK1δ), CaMK4, CK (e.g., CK2), or CaMK2 is given to the patient, but it is particularly advisable in such cases to begin with low doses to determine if adverse side effects are experienced.

The present invention also relates to kits. Without being so limited, it relates to kits for stratifying scoliotic subjects and/or predicting whether a subject is at risk of developing a scoliosis comprising an isolated nucleic acid, a protein or a ligand such as an antibody in accordance with the present invention as described above. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the subject sample (DNA genomic nucleic acid, cell sample or blood samples), a container which contains in some kits of the present invention, the probes used in the methods of the present invention, containers which contain enzymes, containers which contain wash reagents, and containers which contain the reagents used to detect the extension products. Kits of the present invention may also contain instructions to use these probes and or antibodies to stratify scoliotic subjects or predict whether a subject is at risk of developing a scoliosis.

The articles “a,” “an” and “the” are used herein to refer to one or to more than one (i.e., to at least one) of the grammatical object of the article.

The term “including” and “comprising” are used herein to mean, and are used interchangeably with, the phrases “including but not limited to” and “comprising but not limited to”.

The terms “such as” are used herein to mean, and is used interchangeably with, the phrase “such as but not limited to”.

The term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term “about”.

As used herein, the term “purified” or “isolated” refers to a molecule (e.g., polynucleotide or polypeptide) having been separated from a component of the composition in which it was originally present. Thus, for example, an “isolated polynucleotide” or “isolated polypeptide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. For the sake of brevity, the units (e.g., 66, 67 . . . 81, 82, 83, 84, 85, . . . 91, 92% . . . ) have not been specifically recited but are considered nevertheless within the scope of the present invention.

The present invention is illustrated in further details by the following non-limiting examples.

### Example 1

### Materials and Methods

### French-Canadian Patients (Montreal's Cohort)

This study was approved by the institutional review boards of The Sainte-Justine University Hospital, The Montreal Children's Hospital, and The Shriners Hospital for Children. Three populations including children with AIS, families of children with AIS and control subjects were enrolled in the study. Healthy children recruited in Montreal's elementary schools and Trauma cases were used as controls. The recruitment was approved by the Montreal English school Board, The Affluent School Board and all institutional review Board mentioned above. Parents or legal guardians of all participants with or without AIS gave their informed written consent, and minors gave their assent. All participants were examined by one of the seven orthopedic surgeons (H.L., B.P., C-H.R., G.G., J.O., M.B-B., S.P.) participating in this study.

### Italian Patients (Milano's Cohort)

A total of 139 consecutive AIS patients and 103 controls subjects were enrolled at the IRCCS Istituto Ortopedico Galeazzi (Milano, Italy) and represent a replication cohort. This replication study was approved by the institutional review board of IRSCC and The Sainte-Justine University Hospital. All patients were examined by one orthopedic surgeon (M.B-B).

### Cell Preparation and Culture

Osteoblasts were isolated from bone specimens obtained intraoperatively from vertebras (varying from T3 to L4 according to the surgical procedure performed) and from other anatomical sites (tibia or femur) in AIS patients and trauma control cases, respectively as previously described (Moreau et al., 2004).

Myoblasts were isolated from biopsy specimen of skeletal muscle obtained from AIS and trauma control patients. Each biopsy specimen was cleared of remains fatty and connective tissue prior to being cut into smaller pieces. The tissue pieces were then transferred into PBS solution containing 0.01% collagenase, and digested for 45 min at 37° C. After dilution with culture media (1:1), the solution was filtered in sterile conditions through a nylon filter of 45 μm prior to the centrifugation at 280×g for 5 min at room temperature. The pallet containing myoblasts was suspended in culture media (alpha-MEM) supplemented with 20% foetal bovine serum (certified FBS; Invitrogen, Burlington, ON, Canada), and 1% penicillin/streptomycin (Invitrogen). After two weeks, culture media was replaced by fresh culture media supplemented with 10% FBS and 1% penicillin/streptomycin and myoblasts were allowed to grow until confluence.

Peripheral blood mononuclear cells (PBMC) were extracted from blood obtained from patients and control groups, as previously described (Akoume et al., 2010).

### Functional Classification

Patients were classified by evaluating the functional status of melatonin signaling in PBMC with cellular dielectric spectroscopy (CDS), using CellKey™ apparatus, as previously described (Akoume et al., 2010 and WO 2010/040234, 2010 to Moreau et al.). The impedance that reflects the cellular changes resulting from ligand/receptor interaction was measured for 15 minutes to monitor the cellular response to 14⁻⁴M iodomelatonin stimulation. The classification was achieved according to the following value ranges of impedance: between 0 and 40 ohms for FG1, between 40 and 80 ohms for FG2 and between 80 and 120 ohms for FG3. All control cases exhibiting a response extent of less than 120 ohms were excluded from the study.

### Functional Evaluation of G Proteins

The functionality of Gi, Gs and Gq proteins was evaluated by CDS assay in osteoblasts, and the functionality of Gi was also evaluated by CDS assay in myoblasts and PBMCs from the same individuals, using CellKey™ apparatus, as previously described (Akoume et al., 2010 and WO 2010/040234, 2010 to Moreau et al.).

### RNA Interference

All Gi₁, Gi₂, Gi₃, Gs and scrambled siRNA were obtained from Ambion (Ambion USA). The sequences used for gene silencing are shown in Table II below. Osteoblasts from control subjects and AIS patients were transiently transfected in serum-free medium, using Lipofectamine RNAiMAX™ reagent (Invitrogen) according to the manufacturer's instructions and functional experiments were performed 48 h post transfection. The gene knockdown was evaluated by quantitative real-time PCR (qPCR).

TABLE II
SiRNA sequences for Gi₁, Gi₂, Gi₃, Gs

| Gene | | Cat. |
|---|---|---|
| Symbol | SiRNA sequence | Number |
| GNAi1 | GAGAUUGUGGAAAGAUAGUGGUGUA | 1299003 |
| | (SEQ ID NO: 97) | |
| GNAi2 | GAGGACCUGAAUAAGCGCAAAGACA | 1299003 |
| | (SEQ ID NO: 98) | |
| GNAi3 | UCAGCUCAAUGAUUCUGCUUCAUAU | 1299003 |
| | (SEQ ID NO: 99) | |
| GNAS | ACAACAUGGUCAUCCGGGAGGACAA | 1299003 |
| | (SEQ ID NO: 100) | |

### Quantitative Real-Time PCR

RNA was isolated from osteoblasts using TRIzol™ reagent (Invitrogen) according to the manufacturer's protocol. Total RNA (1 μg) was reverse-transcribed into cDNA using Tetro™ cDNA synthesis Kit (Bioline). Following cDNA synthesis, qPCR was performed using a PCR master mix containing QuantiTect™ SYBR Green PCR Master Mix (QIAGEN, Ontario, Canada). Transcript expression was assessed with the Stratagene™ Mx3000P (Agilent Technologies, La Jolla, Calif.) and calculations were performed according to the ΔΔCT method using β-actin as internal control. The sequences of the forward and reverse primers used for identification of human mRNA of our interest genes are shown in Table Ill.

TABLE III
List of the forward and reverse sequences

| | |
|---|---|
| Forwards | Reverses |

| | | |
|---|---|---|
| Gi1 | AGGGCTATGGGGAGGTTGAAGAT | ACTCCAGCAAGTTCTGCAGTCA |
| | (SEQ ID NO: 1) | (SEQ ID NO: 2) |
| Gi2 | AGGGAATACCAGCTCAACGACTCA | TGTGTGGGGATGTAGTCACTCTGT |
| | (SEQ ID NO: 3) | (SEQ ID NO: 4) |
| Gi3 | GAGAGTGAAGACCACAGGCATT | CGTTCTGATCTTTGGCCACCTA |
| | (SEQ ID NO: 5) | (SEQ ID NO: 6) |
| Gs | GAGACCAAGTTCCAGGTGGACA | GATCCACTTGCGGCGTTCAT |
| | (SEQ ID NO: 7) | (SEQ ID NO: 8) |
| Gq | ATCAGAACATCTTCACGGCC | AAAGCAGACACCTTCTCCAC |
| | (SEQ ID NO: 9) | (SEQ ID NO: 10) |
| PKC-α (PKCA) | CGGAATGGATCACACTGAGAAG | ACATAAGGATCTGAAAGCCCG |
| | (SEQ ID NO: 11) | (SEQ ID NO: 12) |
| PKC-β (PKCB) | TTCCCGATCCCAAAAGTGAG | GTCAAATCCCAATCCCAAATCTC |
| | (SEQ ID NO: 13) | (SEQ ID NO: 14) |
| PKC-δ (PKCD) | GCTTCAAGGTTCACAACTACATG | ACCTTCTCCCGGCATTTATG |
| | (SEQ ID NO: 15) | (SEQ ID NO: 16) |
| PKC-ϵ (PKCE) | CCTACCTTCTGCGATCACTG | TACTTTGGCGATTCCTCTGG (SEQ |
| | (SEQ ID NO: 17) | ID NO: 18) |
| PKC-η (PKCHt) | GTAAATGCGGTGGAACTTGC | ACCCCAATCCCATTTCCTTC |
| | (SEQ ID NO: 19) | (SEQ ID NO: 20) |
| PKC-I (PKCI) | GAGAAGCATGTGTTTGAGCAG | GGAAGTTTTCTTTGTCGCTGC |
| | (SEQ ID NO: 21) | (SEQ ID NO: 22) |
| PKC-γ | ACGAAGTCAAGAGCCACAAG | GTCGATGAACCACAAAGCTG |
| (Gamma) | (SEQ ID NO: 23) | (SEQ ID NO: 24) |
| PKC-θ (PKCQ) | CGGATTTTGGAATGTGCAAGG | CATAAAGGAGAACCCCGAAGG |
| | (SEQ ID NO: 25) | (SEQ ID NO: 26) |
| PKC-ζ (PKCZ) | TGCTTACATTTCCTCATCCCG | CGCCCGATGACTCTGATTAG (SEQ |
| | (SEQ ID NO: 27) | ID NO: 28) |
| CaMK1-δ | AATGGAGGGCAAAGGAGATG | AAGATGTAGGCAATCACTCCG |
| (CaM K1D) | (SEQ ID NO: 29) | (SEQ ID NO: 30) |
| CaMK1-γ | CTTGAGAAGGATCCGAACGAG | TTGCCTCCACTTGCTCTTAG |
| (CaMK1G) | (SEQ ID NO: 31) | (SEQ ID NO: 32) |
| CaMK2-α | CAGTTCCAGCGTTCAGTTAATG (SEQ | TTCGTGTAGGACTCAAAATCTCC |
| (CaMK2A) | ID NO: 33) | (SEQ ID NO: 34) |
| CaMK2-β | CTCTGACATCCTGAACTCTGTG | CCGTGGTCTTAATGATCTCCTG |
| (CaMK2B) | (SEQ ID NO: 35) | (SEQ ID NO: 36) |
| CaMK2-δ | GGCACACCTGGATATCTTTCTC | AGTCTGTGTTGGTCTTCATCC |
| (CaMK2D) | (SEQ ID NO: 37) | (SEQ ID NO: 38) |
| CaMK2-γ | AAACAGTCTCGTAAGCCCAG | ATCCCATCTGTAGCGTTGTG |
| (CaMK2G) | (SEQ ID NO: 39) | (SEQ ID NO: 40) |
| CaMK4 | TCGCCTCTCACATCCAAAC | CATCTCGCTCACTGTAATATCCC |
| | (SEQ ID NO: 41) | (SEQ ID NO: 42) |
| CaMK2n1 | AGGACACCAACAACTTCTTCG | GGTGCCTTGTCGGTCATATT |
| | (SEQ ID NO: 43) | (SEQ ID NO: 44) |
| PKA-α1 | CTCAGTTCCTGGAGAAAGATGG | CCCAGTCAATTCATGTTTGCC |
| | (SEQ ID NO: 45) | (SEQ ID NO: 46) |
| PKA-α2 | ATGGAATATGTGTCTGGAGGTG | TGGTTTCAGGTCTCGATGAAC |
| | (SEQ ID NO: 47) | (SEQ ID NO: 48) |
| PKA-β1 | GAGTAAACTTCCCCTCACCAG | TCCACTGACCATCCACAAAG |
| | (SEQ ID NO: 49) | (SEQ ID NO: 50) |
| PKA-β2 | CACTGTTATCCGCTGGTCTG | CTTGTATTGGTGCTCTCCCTC |
| | (SEQ ID NO: 51) | (SEQ ID NO: 52) |
| PKA-cα | CAAGGACAACTCAAACTTATACATGG | CAGATACTCAAAGGTCAGGACG |
| | (SEQ ID NO: 53) | (SEQ ID NO: 54) |
| PKA-cβ | CCTTTCCTTGTTCGACTGGAG | TGAGCTGCATAGAACCGTG |
| | (SEQ ID NO: 55) | (SEQ ID NO: 56) |
| PKA-cγ | CCGGATCTCCATCAATGAGAAG | TTCAATCCAACCCTCCCATC |
| | (SEQ ID NO: 57) | (SEQ ID NO: 58) |
| PKA-γ1 | GCGCATTCTGAAGTTCCTCA | AAAATCCCCAGAGCCACATAG |
| | (SEQ ID NO: 59) | (SEQ ID NO: 60) |
| PKA-γ2 | TTGCCCGTTATTGACCCTATC | CGTTCCTATTCCAAGCTCATCC |
| | (SEQ ID NO: 61) | (SEQ ID NO: 62) |
| PKA-γ13 | TGACTGCACTGGACATCTTTG | TGGTTGTAGGTTTGCTGGG |
| | (SEQ ID NO: 63) | (SEQ ID NO: 64) |
| CK-1α | TGTCGGAGGGAAATATAAACTGG | GGCCTTCTGAGATTCTAGCTTC |
| | (SEQ ID NO: 65) | (SEQ ID NO: 66) |
| CK-1γ1 | AGGTGGAGGTAGTGGAGG | GTACAATTGAGTCAGAGTCCCC |
| | (SEQ ID NO: 67) | (SEQ ID NO: 68) |
| CK-1γ2 | GTGATGTTCTAGCCACAGAGG | CCCTTTCCCTCCTTTCTTGTC |
| | (SEQ ID NO: 69) | (SEQ ID NO: 70) |
| CK-1γ3 | GTTCAAATGCACCCATCACAG | AGTAACTCCCCAGGATCTGTC |
| | (SEQ ID NO: 71) | (SEQ ID NO: 72) |
| CK 2α1 | GTATGAGATTCTGAAGGCCCTG | CCAAACCCCAGTCTATTAGTCG |
| | (SEQ ID NO: 73) | (SEQ ID NO: 74) |
| CK-2α2 | CGATACGACCATCAACAGAGAC | TCGCTTTCCAGTCTTCATCG |
| | (SEQ ID NO: 75) | (SEQ ID NO: 76) |

### G Protein Expression in Osteoblasts

Expression levels of Gi proteins isoforms and Gs protein were determined using standard western blotting technique. In brief, osteoblasts from AIS patients and trauma control cases were lysed in RIPA buffer (25 mM Tris-HCl, pH 7.4, 150 mM NaCl, 1% NP-40, 1% sodium deoxycholate, 0.1% SDS) added with 5 mM NaVO₄ and protease inhibitor cocktail (Roche molecular Biochemicals, Mannheim, Germany). Immunoblots were performed with primary antibodies directed specifically against Gi₁, Gi₂, Gi₃ or Gs (Santa Cruz Biotechnology, Santa Cruz, Calif.) and peroxidase-conjugated secondary antibody. Bands were then visualized using SuperSignal™ chemilunescent substrate (Pierce, Rockford, Ill.).

### Assessment of Phosphorylation of Gi Protein Isoforms in Osteoblasts

Comparative studies were performed to examine the level of phosphorylation and to identify the phosphorylated Gi isoform with immunoprecipitated proteins using isoform specific antibodies and, subsequently, probing the level of phosphorylation with a phospho-serine/threonine specific antibody (Santa Cruz Biotechnology). Whole cell proteins (1 mg) were incubated with anti-Gi₁, anti-Gi₂ or anti-Gi₃ (Santa Cruz Biotechnology), plus protein G beads for immunoprecipitation (IP) at 4° C. overnight. Purified proteins were loaded on 10% gel after IP, then processed by transferring to nitrocellulose membrane and 5% BSA blocking. Membranes were exposed to phospho-serine/threonine specific antibody at 4° C. overnight and, subsequently, treated with secondary antibody at room temperature for 1 h. Bands were visualized using SuperSignal Cheminescence™, and quantified by densitometric scanning.

### Statistical Analysis

Data are presented as mean±SE, and were analyzed by ANOVA or Student's t test using GraphPad™ Prism 4.0 software. Multiple comparisons of means were performed with one-way ANOVA followed by a post-hoc test of Dunnett. Only P values<0.05 were considered significant.

Demographic and Clinical Characteristics of French-Canadian and Italian Cohorts.

The French-Canadian cohorts consisted of 956 consecutive (i.e. without selection: the first 956 subjects having accepted to participate in the study) adolescents with AIS and 240 aged-matched controls without a family history of scoliosis. The absence of spine abnormalities was confirmed in all control subjects, while a total of 162 AIS patients exhibited curvatures greater than 45° and 794 AIS patients had curvature between 10° and 44°. In the Italian cohort, the moderate curves (Cobb angle 10°-44°) were apparent in 61 AIS patients and the severe curves (Cobb angle>45°) in 78 AIS patients. All AIS patients were age-matched with control subjects in both Canadian and Italian cohorts (see Table IV below).

TABLE IV
Demographic data of AIS and healthy control subjects

| | Canadian cohort | | Italian cohort | |
|---|---|---|---|---|
| | N | Mean age (years) | N | Mean age (years) |
| Healthy control | 240 | 12.4 ± 3.2 | 103 | 11.0 ± 1.6 |
| subjects | | | | |

AIS patients

| | | | | |
|---|---|---|---|---|
| Cobb Angle <44% | 794 | 13.0 ± 2.6 | 78 | 13.6 ± 2.8 |
| Cobb Angle >45% | 162 | 15.1 ± 2.1 | 61 | 13.8 ± 2.2 |

### Example 2

### Clinical Outcomes of AIS Patients According to their Functional Classification

Patients were classified according to the response degree of their PBMCs to iodomelatonin stimulation as indicated in Example 1. Of 956 AIS patients from the Canadian cohort, 243 were classified in functional group 1 (FG1), 353 in functional group 2 (FG2) and 360 in functional group 3 (FG3). The prevalence of all three functional groups was comparable among low to moderate cases (Cobb angle 10°-44°). However, the FG2 was predominant among severe cases (Cobb angle>45°) with a proportion of 56% compared to 31% and 13% for FG3 and FG1, respectively. See Table V below.

Similar profile of distribution was observed in the Italian cohort in whom surgery was required for 61% of FG2, 36% of FG3 and 3% of FG1 AIS patients. Collectively, these results strengthen the view that clinical outcomes vary among AIS patients and suggest that the risk of severe progression is higher for FG2, moderate for FG3 and low for FG1. See Table VI below.

TABLE V
Clinical data of AIS patients classified into functional groups
Canadian Cohort

| | Cobb Angle < 44° | | Cobb Angle > 45° | | Curve Type | | |
|---|---|---|---|---|---|---|---|
| | N (%) | Cobb Angle (°) | N (%) | Cobb Angle (°) | Single N (%) | Double N (%) | Triple N (%) |
| FG1 | 222 (28) | 20.0 ± 10.1 | 21 (13) | 60.8 ± 12.4 | 124 (51) | 109 (45) | 10 (4) |
| FG2 | 262 (33) | 21.5 ± 9.5 | 91 (56) | 60.0 ± 10.8 | 170 (48) | 162 (46) | 21 (6) |
| FG3 | 310 (39) | 18.9 ± 9.4 | 50 (31) | 60.7 ± 12.6 | 194 (54) | 148 (41) | 18 (5) |

TABLE VI
Clinical data of AIS patients classified into functional groups
Italian Cohort

| | Cobb Angle < 44° | | Cobb Angle > 45° | | Curve Type | | |
|---|---|---|---|---|---|---|---|
| | N (%) | Cobb Angle (°) | N (%) | Cobb Angle (°) | Single N (%) | Double N (%) | Triple N (%) |
| FG1 | — | — | 1 (4) | 62.0 | 1 (2) | — | — |
| FG2 | 92 (95) | 24.3 ± 10.0 | 15 (65) | 53.5 ± 13.4 | 58 (91) | 48 (87) | — |
| FG3 | 5 (5) | 17.6 ± 5.7 | 7 (30) | 60.6 ± 12.9 | 5 (8) | 7 (13) | — |

### Example 3

### Each Functional Group Represents a Potential Hereditary Trait

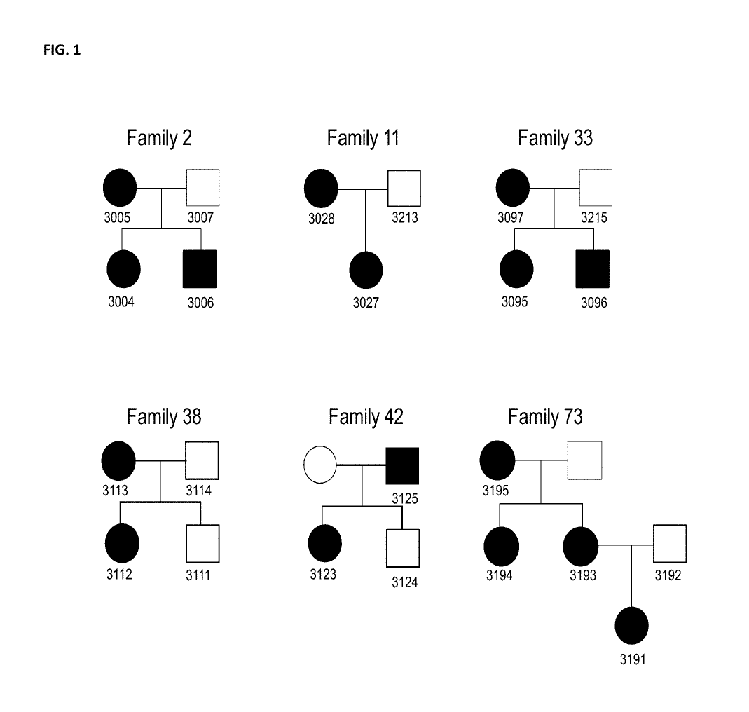

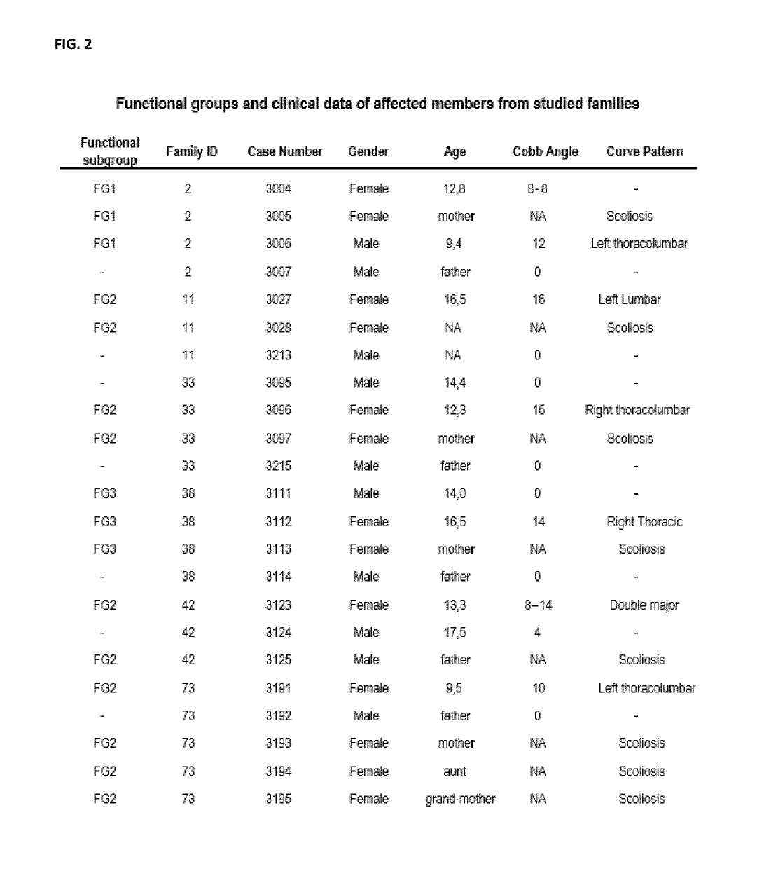

Since the hereditary or genetic basis of AIS has consistently been claimed (Riseborough and Wynne-Davies, 1973); (Blank et al., 1999); (Roach, 1999), the possibility that the biological defect characterizing each functional group may be a hereditary condition was tested. For this purpose, 25 individuals from 6 unrelated families were examined. Pedigrees are shown in FIG. 1. At least two individuals were affected in each family. The classification has revealed that all affected family members belonged to the same functional group and so displayed similar biological defect (FIG. 2). However, neither pattern nor severity of curve was group specific (FIG. 2). This suggests that each functional group represents a biological endophenotype that co-segregates within families independently of curve type and magnitude of spinal deformity.

### Example 4

### Affinity of Melatonin Receptor for its Agonist is Similar Between Functional Groups

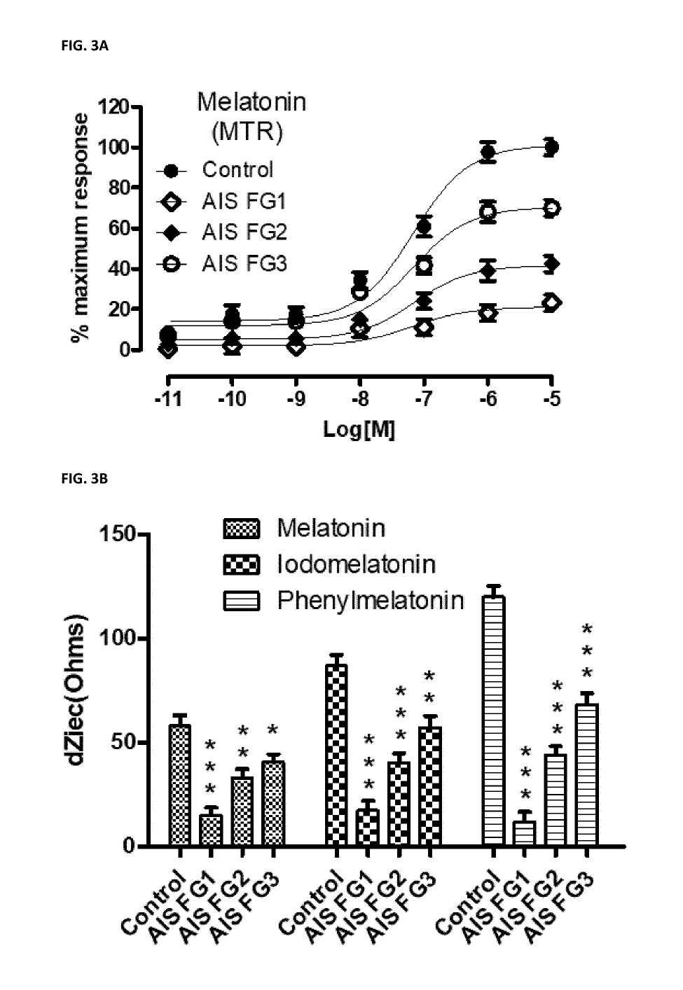

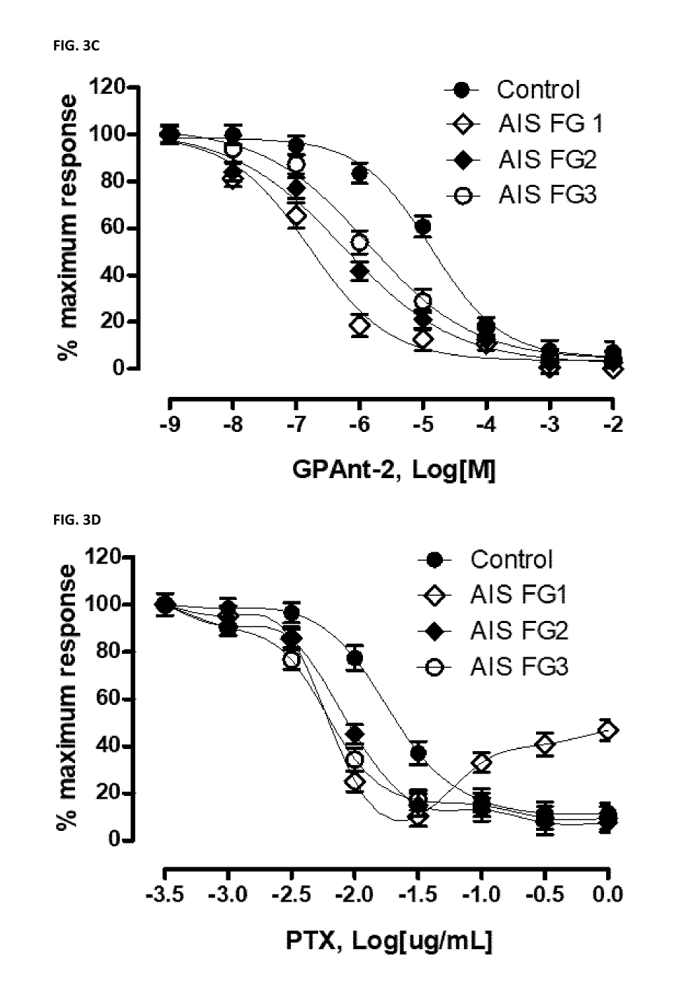

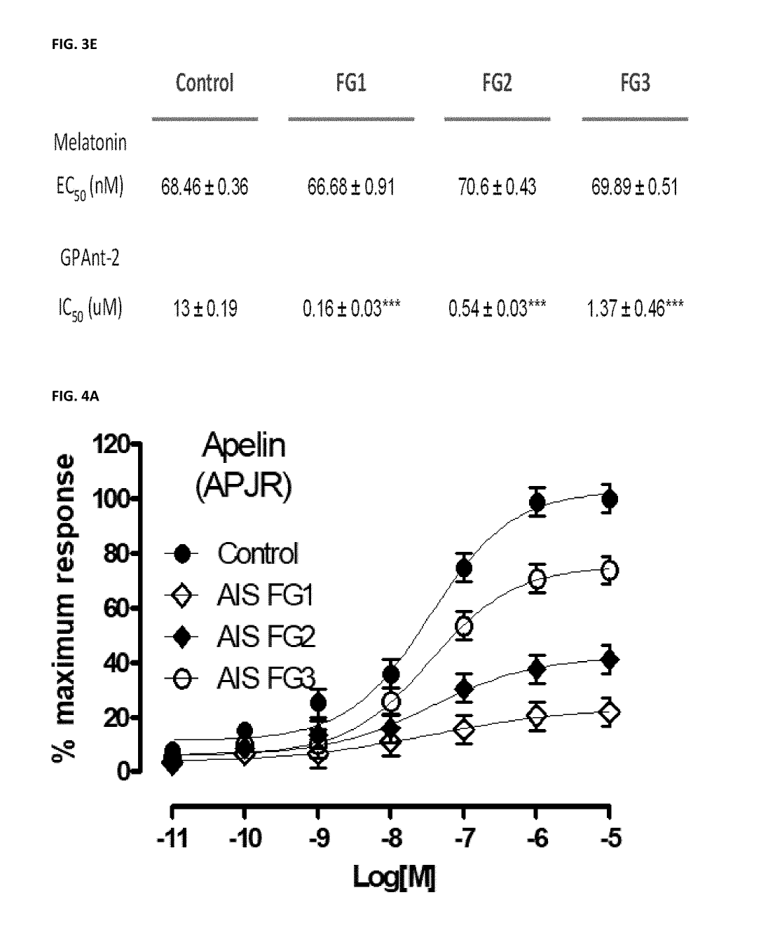

It was tested whether the melatonin signaling dysfunction in AIS was due to changes affecting the melatonin receptor/Gi protein coupling. First was examined whether the affinity of melatonin for its receptors varies among AIS patient groups. For this purpose, concentration-response curves were generated for melatonin using osteoblasts from control subjects and compared with those of AIS patients of each biological endophenotype. As illustrated in FIG. 3A, melatonin caused a concentration-dependent increase of response in all control and AIS groups reaching a plateau at 1 μM, with the higher magnitude in control group and lower but at varying degree within the three AIS groups. The order of magnitude of the maximum response from highest to lowest responses was control group, FG3, FG2 and FG1. Despite this apparent discrepancy in the maximum response between groups, the half-maximum response was observed at similar concentrations in control and AIS groups. These observations suggest that the affinity of melatonin receptor for its agonist is preserved in AIS.

### Example 5

### Activity of Melatonin Receptor in Response to Different Agonists is Comparable Between Control Subjects and AIS Patients

Then was examined the possibility of changes in the activity of melatonin receptor by testing three agonists that have different efficacy to activate melatonin receptor. The response produced with their maximum concentration (10 μM) in osteoblasts from control subjects and AIS patients of each functional group was compared. Results illustrated in FIG. 3B show that the three agonists, melatonin, idomelatonin and phenylmelatonin evoked various degrees of response in osteoblasts from control and AIS subjects. The response to each agonist was lower in all AIS groups compared to the control group. However, the magnitude and the reduction due to AIS in each group reflected the efficacy of agonists, the order of agonist potency being phenylmelatonin>iodomelatonin>melatonin, as previously reported by other investigators using different methods (Nonno et al., 1999). This suggests that the activity of melatonin receptor is comparable between control subjects and AIS patients and underscore that deficit is beyond the melatonin receptor.

### Example 6

### Ability of Agonist/Receptor Complex to Activate Gi Proteins is Decreased in AIS Patients Vs. in Controls

The possibility that the deficit in melatonin signaling could be related to a decreased ability of agonist/receptor complex to activate Gi proteins was tested and results are shown in FIG. 3A-FIG. 3E. The G protein antagonist GPAnt-2, a hydrophobic peptide, has been shown to inhibit receptor/Gi protein coupling by competing with the activated receptor for interaction with G proteins (Mukai et al., 1992). Results illustrated in FIG. 3C show that GPAnt-2 inhibited the response to melatonin stimulation in a concentration-dependent manner in control and AIS functional groups. At maximal concentration, the extent of reduction was similar in all groups, but at submaximal concentrations, the pattern is clearly different. Concentration-response curves of all three AIS functional groups exhibited a left-shift compared to that of the control group, and the IC50 values were significantly decreased among AIS groups when compared to controls. Taking into account that GPAnt-2 competes with receptors on various G proteins, the amount of Gi proteins was further selectively decreased by incubating osteoblasts with increasing concentration of pertussis toxin (PTX). It was found that PTX treatment reduced the cellular response to melatonin in FG2 and FG3 osteoblasts exhibiting a pattern similar to the one obtained with GPAnt-2. In contrast, PTX treatment enhanced the response to melatonin in FG1 osteoblasts at maximal concentration (FIG. 3D). These data show that the reduced melatonin signaling in AIS is most likely due to a decrease in the sensibility of Gi proteins for melatonin receptors, and indicate that the melatonin receptors may also interact with Gs in AIS patients classified in FG1.

### Example 7

### AIS Subjects have a Systemic and Generalized Impairment of Gi Protein-Mediated Receptor Signaling

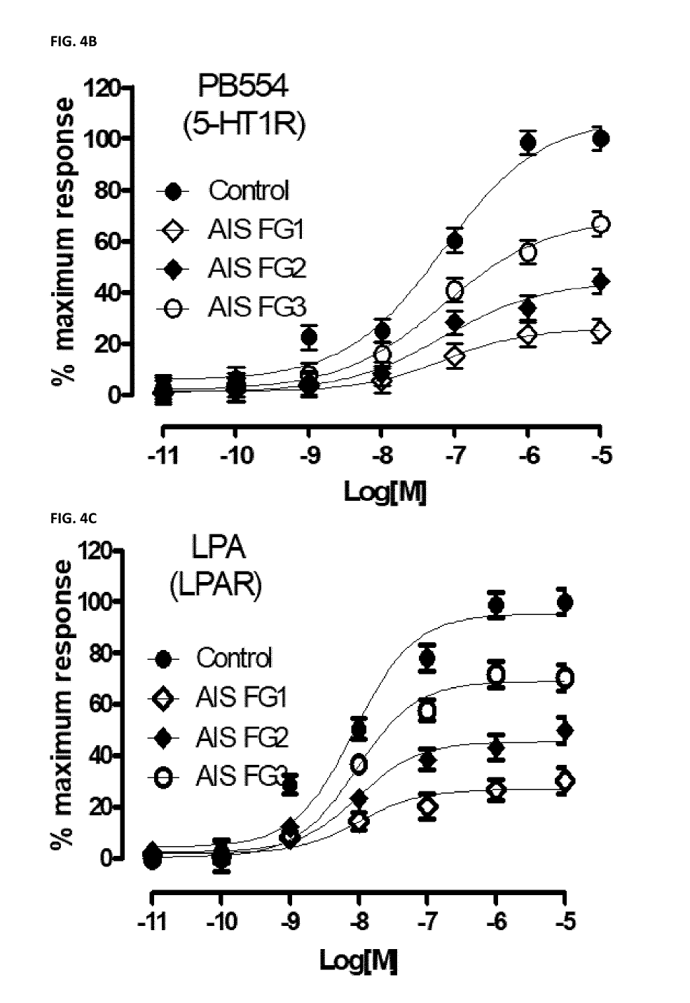

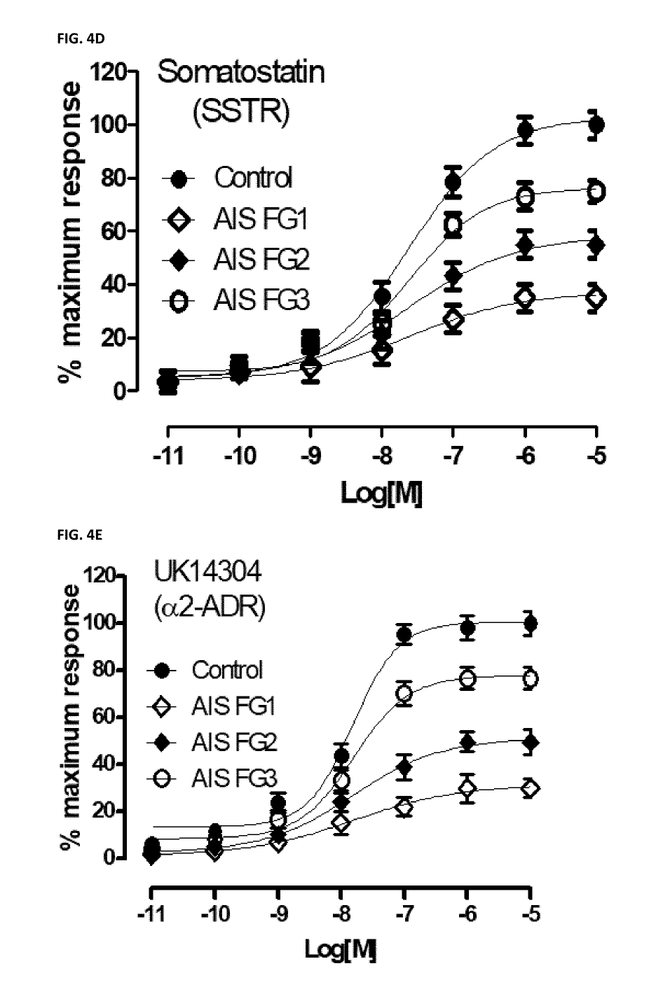

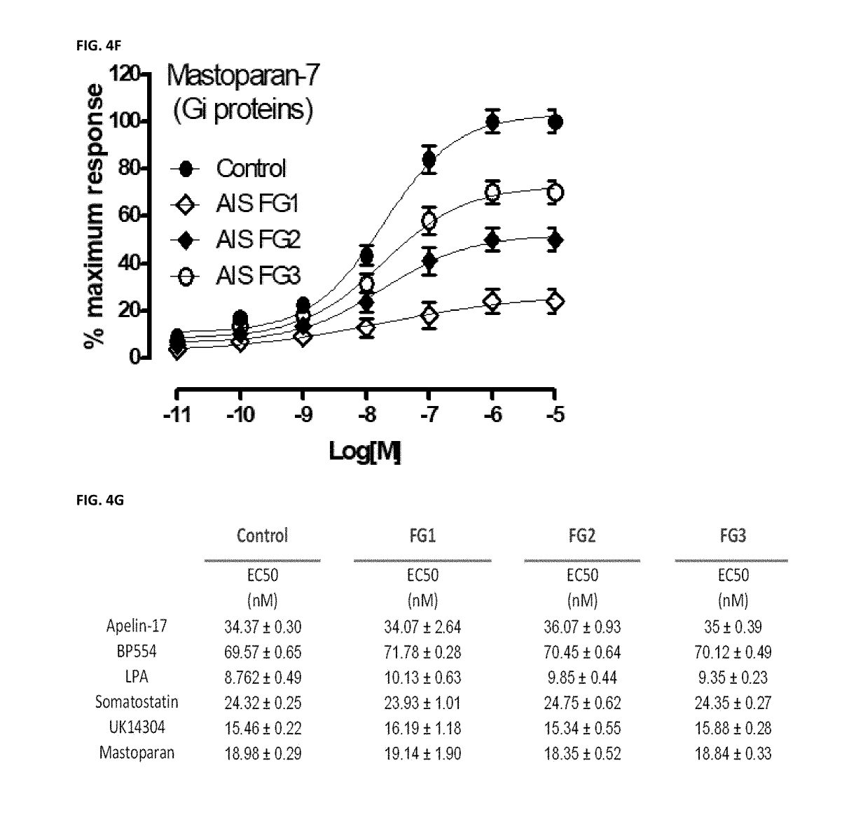

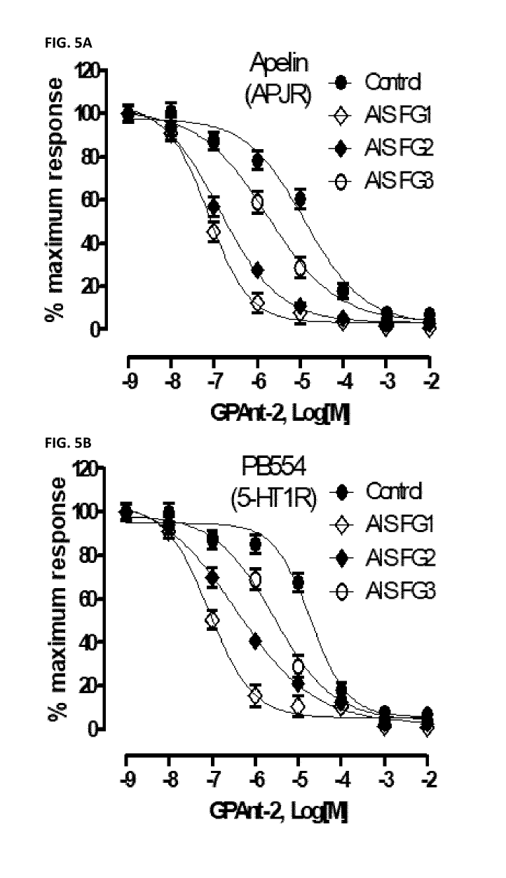

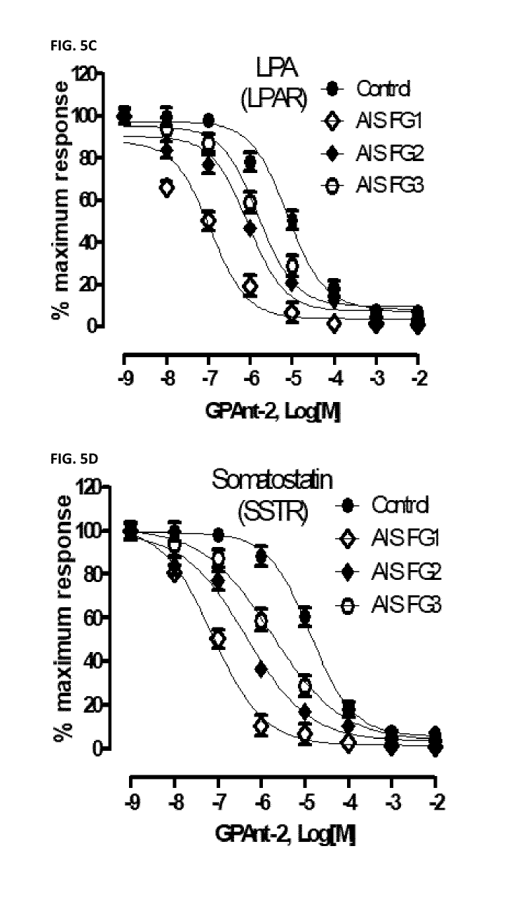

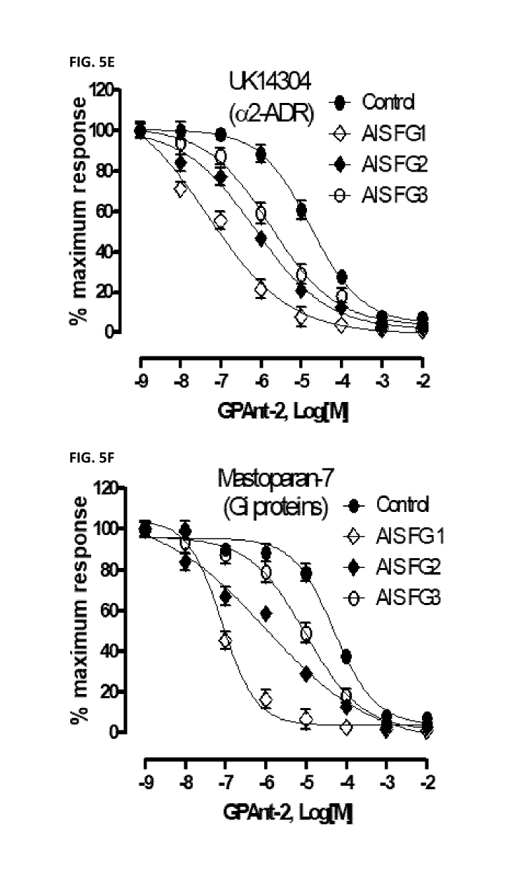

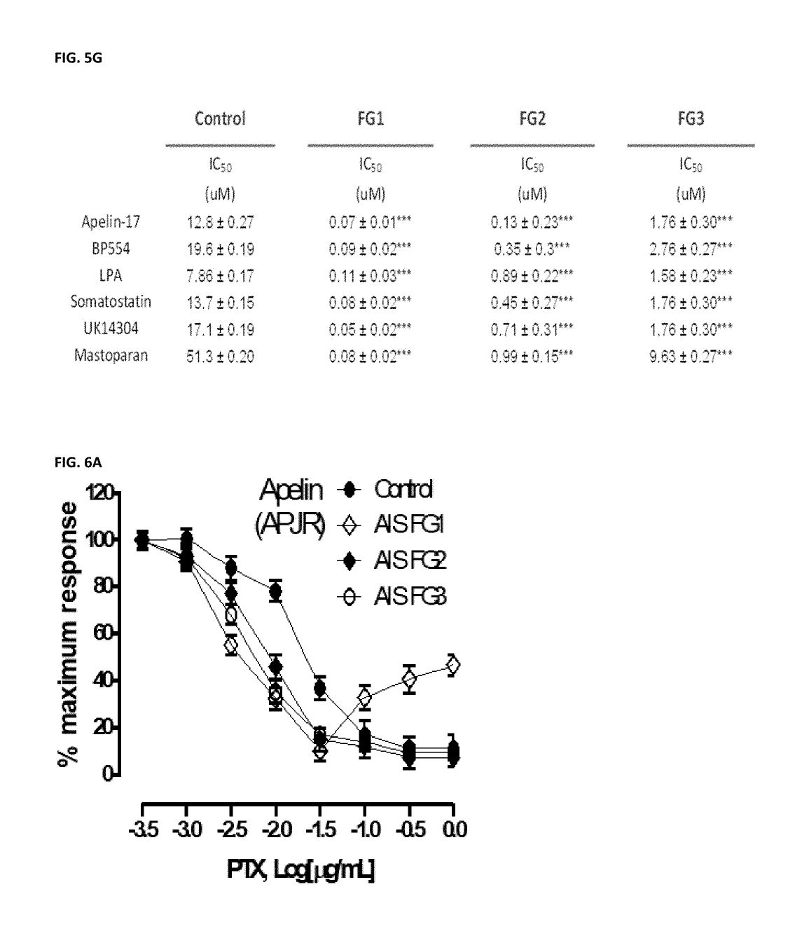

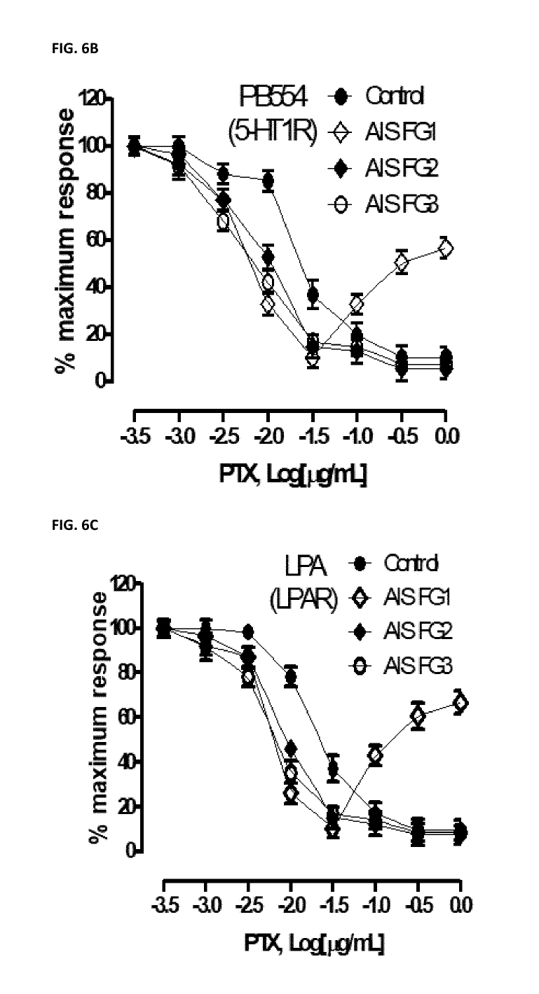

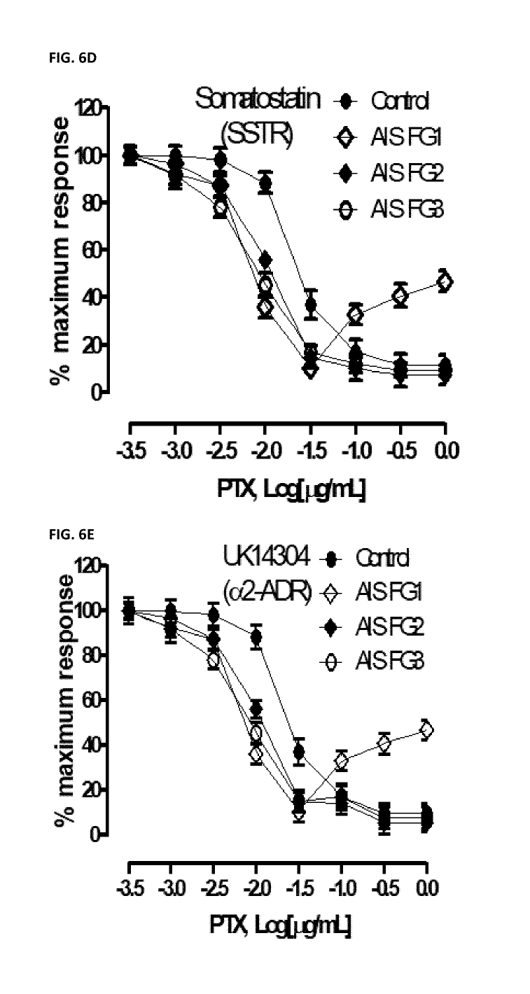

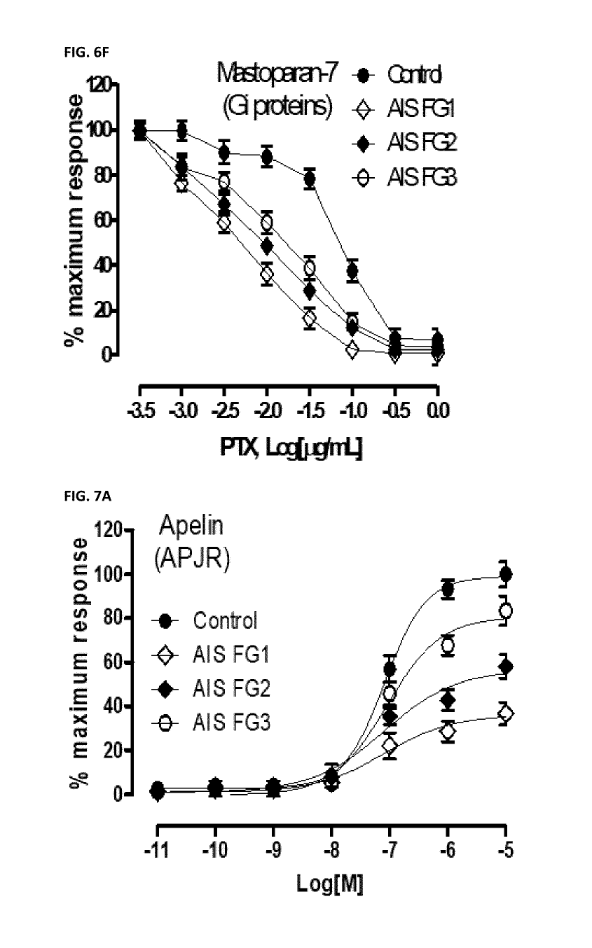

To determine if the signaling dysfunction through Gi proteins as measured in AIS osteoblasts was restricted to melatonin receptors, a comparative study was performed with various synthetic compounds activating selectively other receptors coupled to Gi proteins. Five compounds were used including apelin-17, PB554 maleate, lysophosphatidic acid (LPA), UK14304 and somatostatin to activate endogenous APJ, serotonin 5-HT_{1A}, LPA₁, alpha2-adrenergic and somatostatin (sst) receptors, respectively. As illustrated in FIG. 4A-FIG. 4F, all tested agonists caused a concentration-dependent increase of the cellular response, reaching a plateau at the same concentration in all control and AIS functional groups. In each case, the magnitude of the signaling response was lower in all AIS groups when compared with the control group, but the EC50 values were almost identical in all groups indicating that the affinity of all tested agonists for their respective receptors is not affected in AIS (FIG. 4G). Interestingly, inhibition curves of GPAnt-2 (FIG. 5A-FIG. 5F) or PTX (FIG. 6A-FIG. 6F) generated with any of the five synthetic agonists tested revealed curve patterns similar to those obtained with melatonin. In each case, GPAnt-2 reduced the IC50 values in AIS groups when compared to control group (FIG. 5G). Similar responses were also observed when GPAnt-2 was competed with mastoparan-7 (FIG. 5F), which directly activates Gi proteins by mimicking agonist-activated receptor (Higashijima et al., 1990). Moreover, response to mastoparan was almost abolished in all AIS and control groups following treatment with high concentrations of PTX (FIG. 6F), further pointing to an abnormality at the level of Gi proteins. Collectively, these data strengthen the concept that agonist/receptor interaction is not affected in AIS, and reveal that AIS patients can be functionally stratified with any compounds that activate Gi protein-mediated signaling pathways.

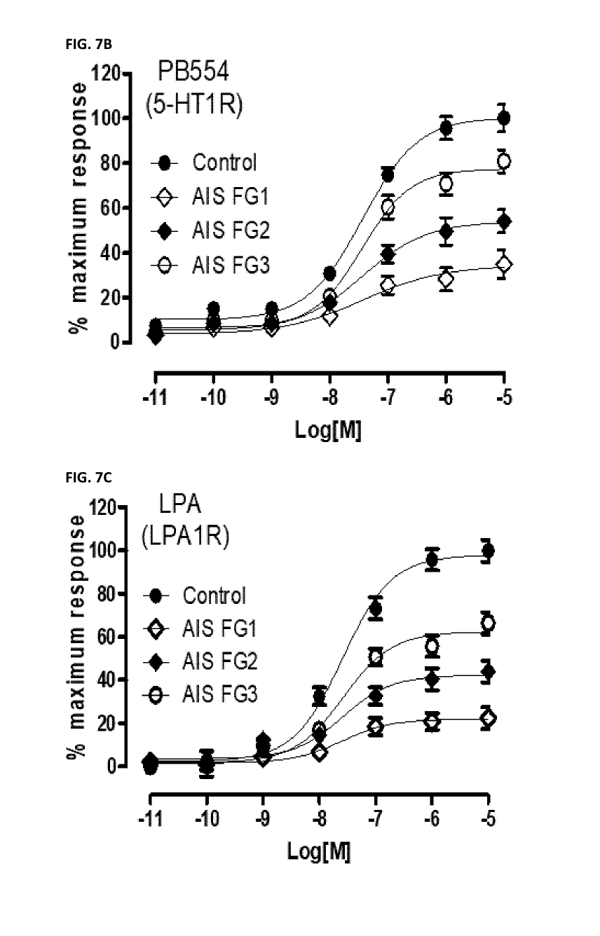

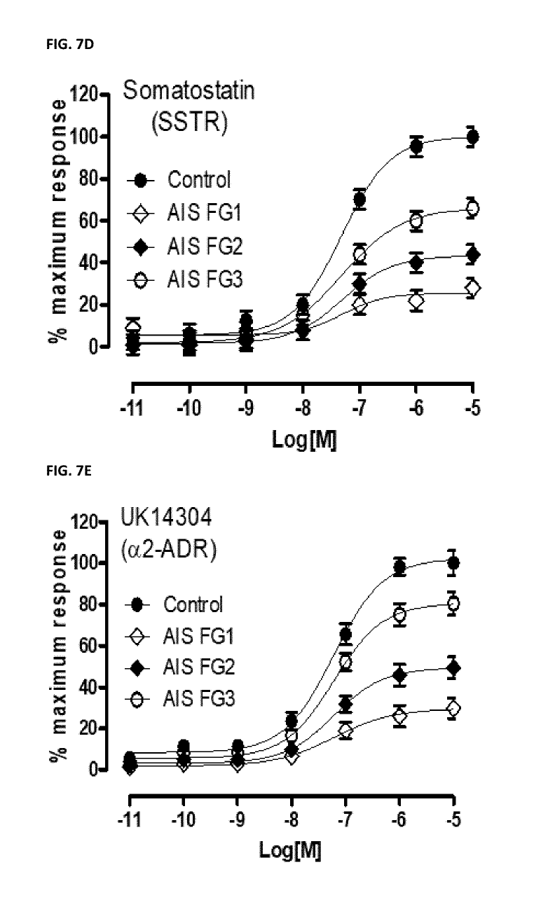

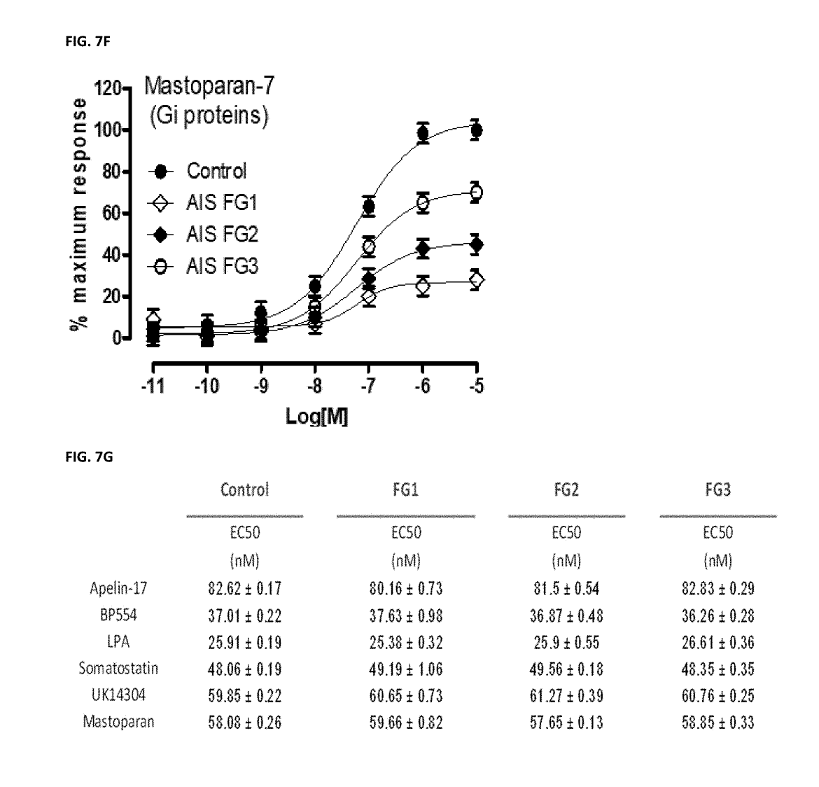

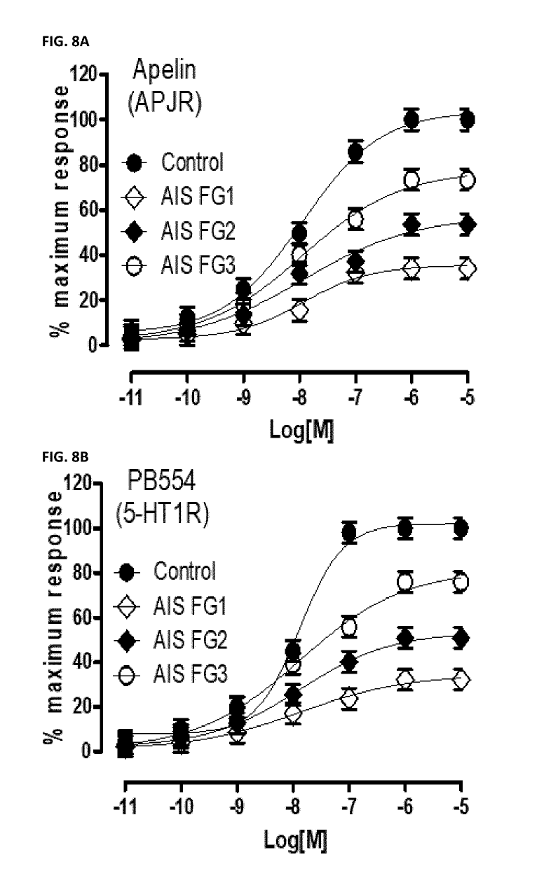

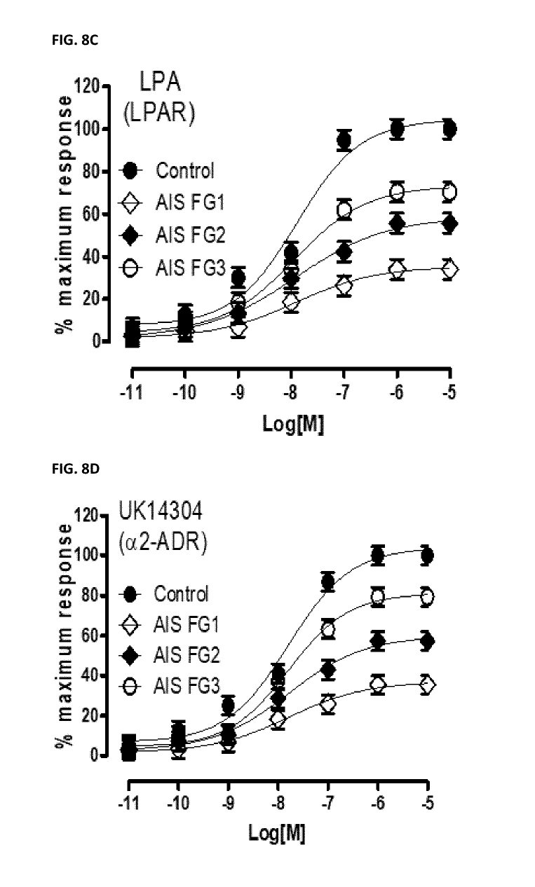

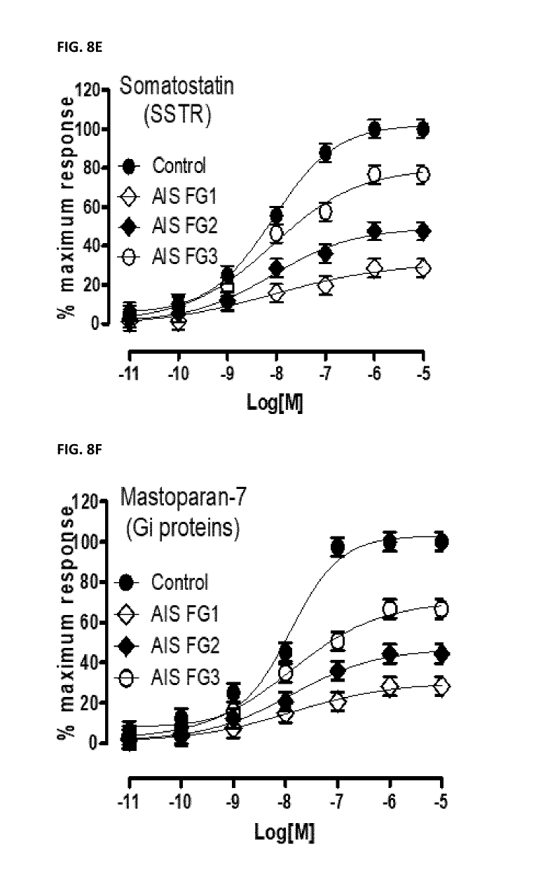

The analysis was extended to other cell types, namely skeletal myoblasts and peripheral blood mononuclear cells (PBMCs) from the same set of controls and AIS patients. A pattern of response similar to that obtained in osteoblasts for each agonist tested was obtained in these cell types (FIG. 7A-FIG. 7G and FIG. 8A-FIG. 8G). Overall, these findings are strongly indicative of a systemic and generalized impairment of Gi protein-mediated receptor signaling.

### Example 8

### Reduction in Gi Protein Function Selectively Influences Gs Protein Function in AIS

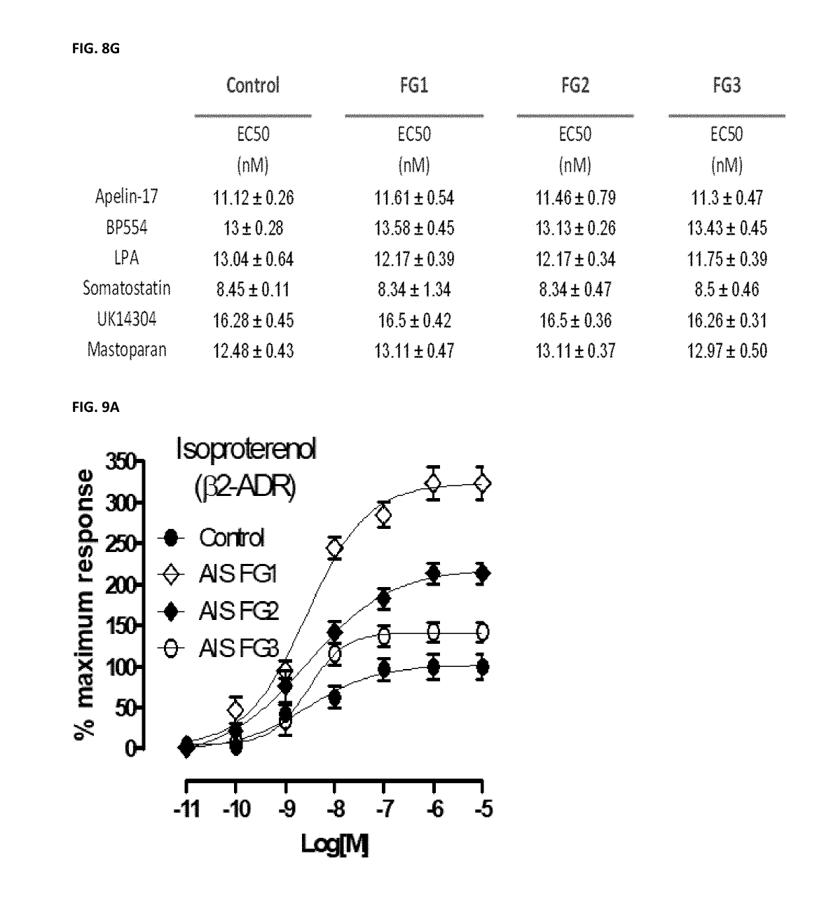

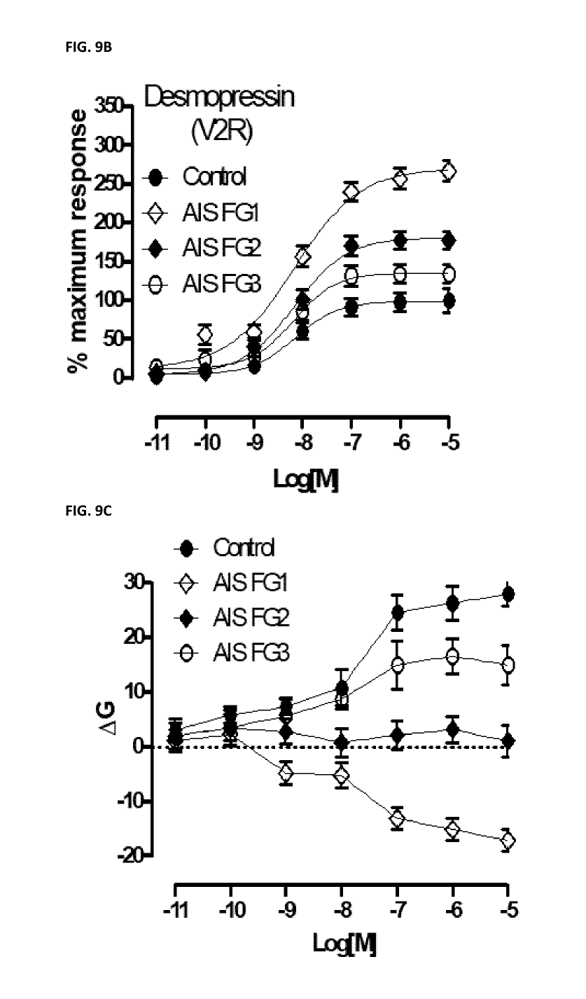

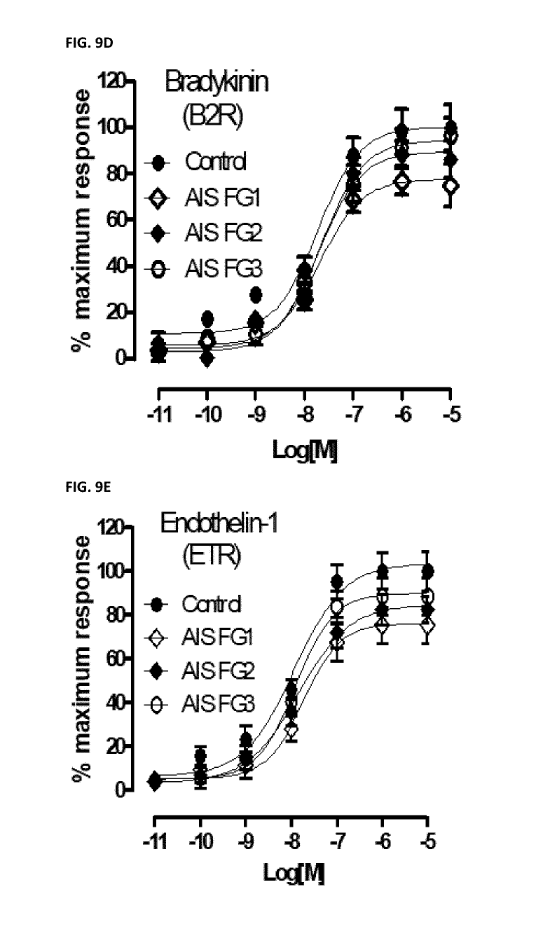

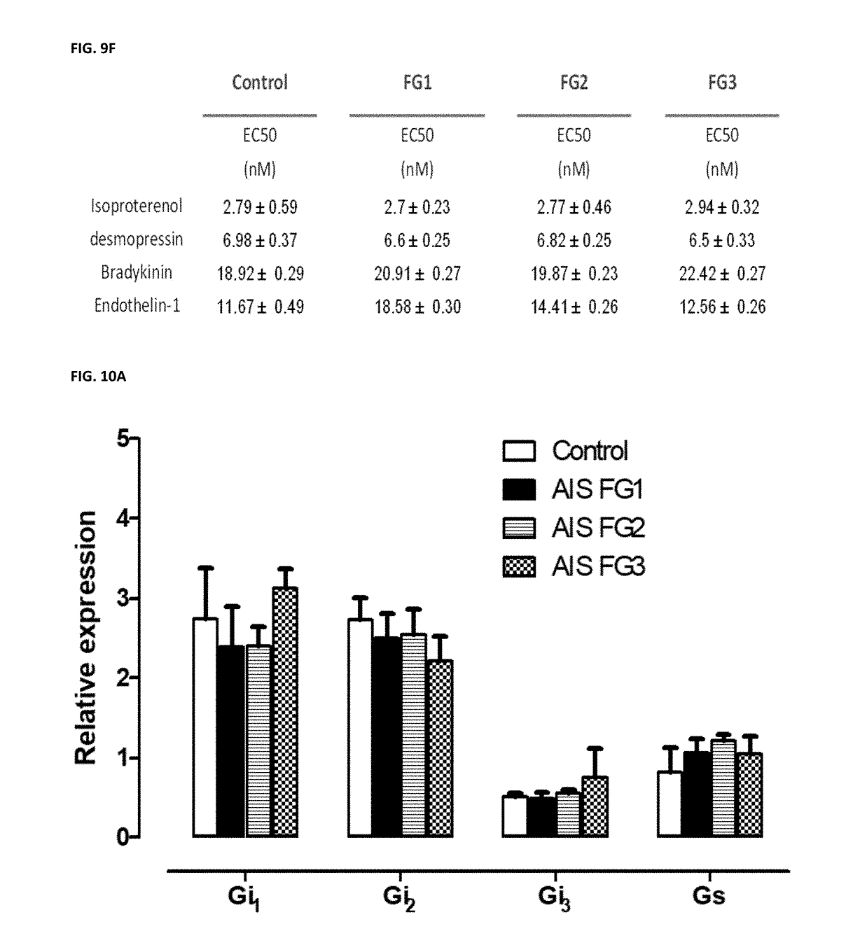

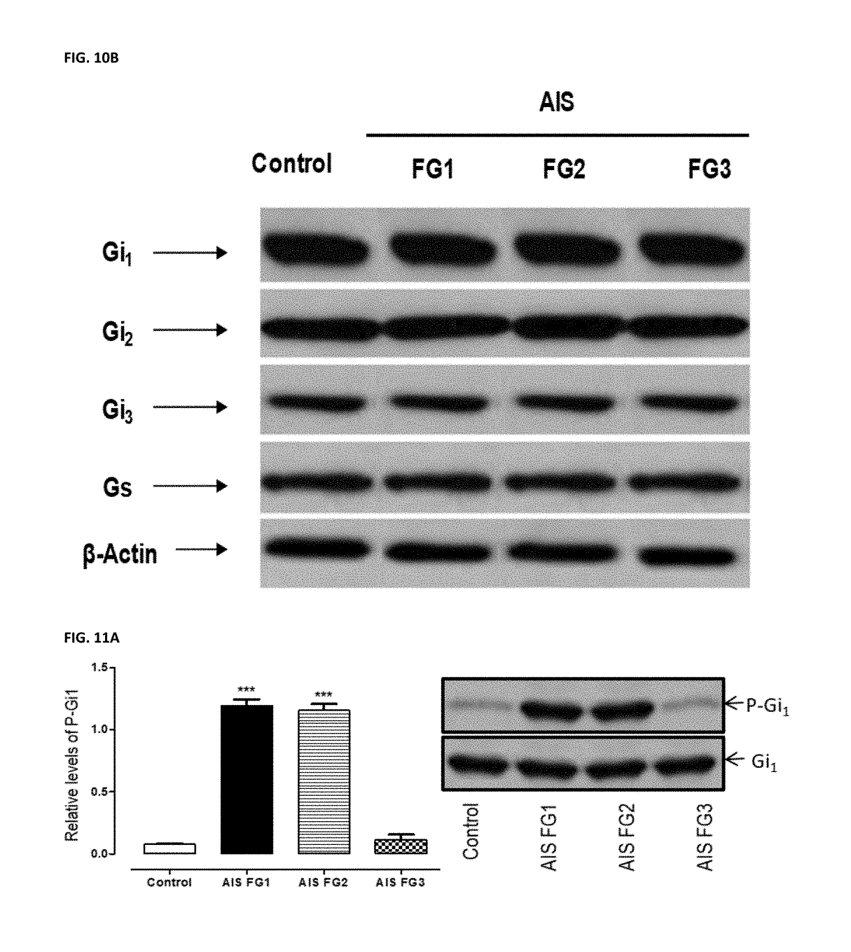

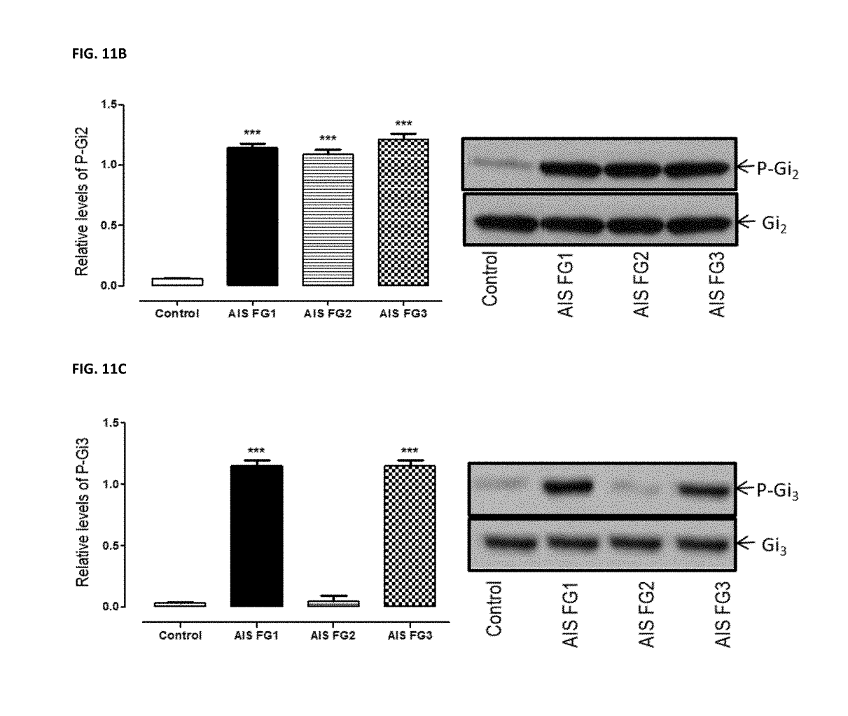

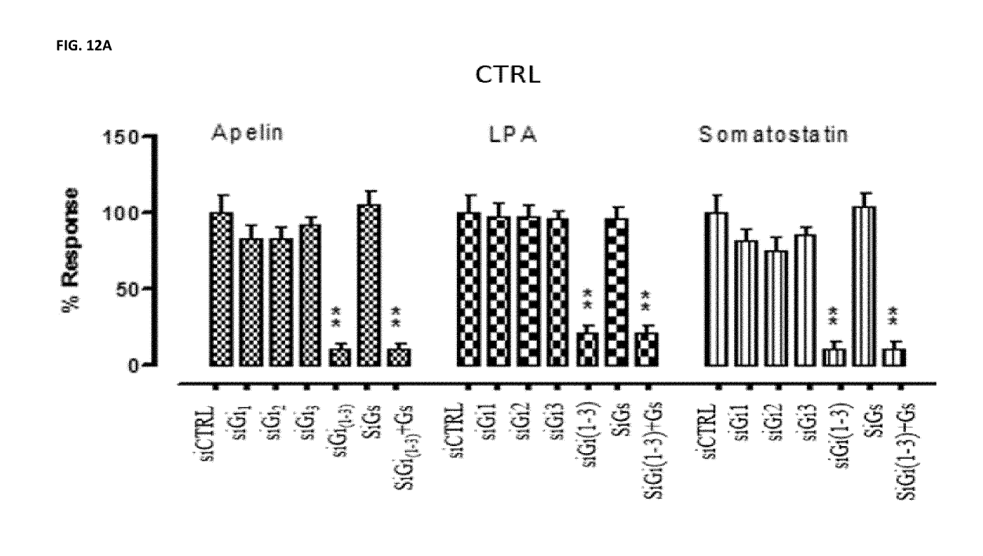

Osteoblasts from control and AIS patients were screened for their response to isoproterenol and desmopressin, which activate beta-adrenergic and vasopressin (V2) receptors, respectively. Both receptors mediate signal transduction through Gs proteins. Results illustrated in FIG. 9A-FIG. 9F show that cellular responses initiated by both agonists were significantly enhanced in osteoblasts from AIS patients when compared with control osteoblasts. For each functional group, increased response to Gs stimulation inversely mirrored the reduced response induced after Gi protein stimulation, suggesting a functional imbalance between Gi and Gs proteins. To further illustrate this divergence, values of the responses to melatonin and isoproterenol were reported as differences (Δ) between response to Gi and Gs protein stimulation. As illustrated in FIG. 9C, response to Gi stimulation predominated in a concentration-dependent manner in control group (i.e., a Gi/Gs ratio of more than about 1.5). A similar pattern was observed in the FG3 group (i.e., a Gi/Gs ratio of more than about 1.5), while no apparent imbalance was observed in the FG2 group (i.e., a Gi/Gs ratio of between about 0.5 and 1.5). In contrast, the FG1 group exhibited predominance for response to Gs stimulation (i.e., a Gi/Gs ratio of less than about 0.5). These data indicate that Gs protein is functionally affected in AIS according to the aberration degree of Gi protein function, revealing a profile of imbalance between Gi and Gs protein function specific to each AIS group.

Results presented reveal a relationship between reduced Gi and increased Gs protein functions in AIS. The profile of the functional imbalance between Gi and Gs protein was specific to each AIS group, indicating that AIS patients can be clearly classified with respect to the profile of imbalance between response to Gi and Gs protein stimulation. Such an approach advantageously eliminates the necessity to use control subjects and allows the monitoring patient responses over time.

The functional status of Gq protein was then studied. Osteoblasts were stimulated with bradykinin and endothelin-1. Both agonists elicited similar responses at various concentrations in osteoblasts derived from control subjects and AIS patients, demonstrating that receptor signaling through Gq protein remains largely intact in AIS (FIG. 9D-FIG. 9E). It appears that reduction in Gi protein function in AIS exclusively influences Gs protein function.

### Example 9

### Differences in the Degree of Response Characterizing the Three AIS Biological Endophenotypes are not Due to Differential Expression of Gi Proteins

The three isoforms of Gi proteins, termed Gi₁, Gi₂ and Gi₃ share the same proprieties and most membranous receptors that interact with Gi proteins are able to initiate signals via each of these isoforms. However, the amplitude of the response depends on the capacity of Gi isoforms to mediate the signal transduction while some Gi isoforms seem more efficient than others.

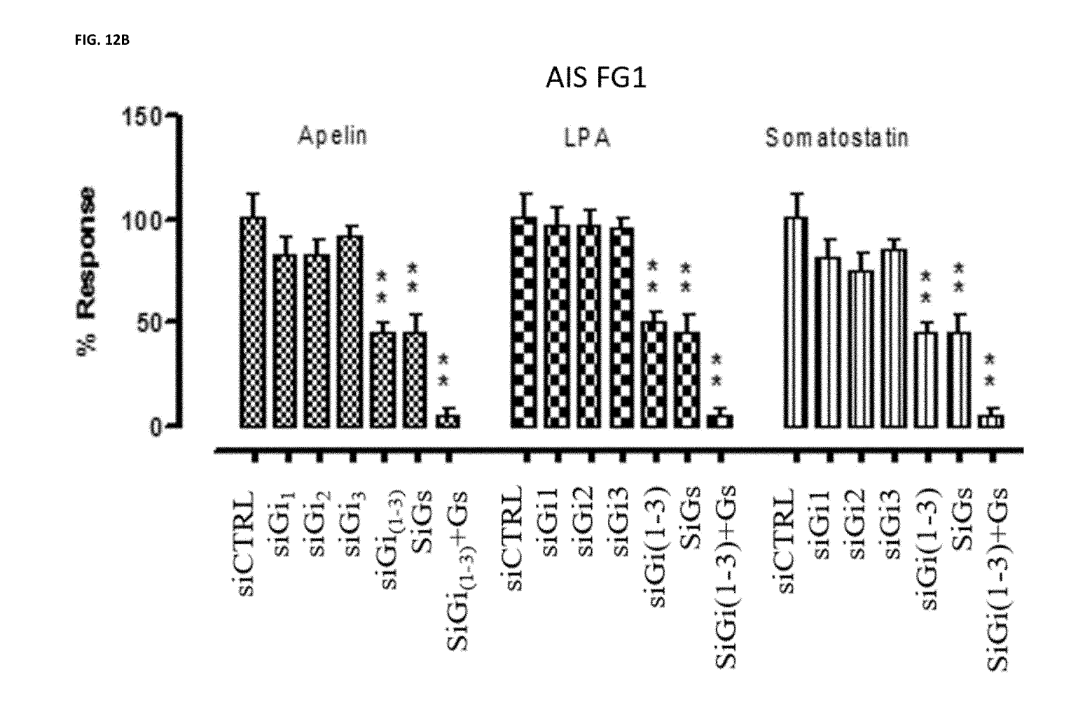

It was tested whether the differences in the degree of response characterizing the three AIS biological endophenotypes is due to the differential alteration of Gi proteins isoforms. First was examined whether observed functional changes are due to changes in the expression of G proteins. The qPCR analysis revealed no significant change in expression of any isoform of Gi proteins (Gi₁, Gi₂ and Gi₃) and Gs protein between control and AIS osteoblasts (FIG. 10A). Assuming that PCR-amplification of the different isoforms was equally efficient, it appears that Gi₁ and Gi₂ were the most abundant isoforms of Gi proteins in control and AIS osteoblasts, while the expression level of Gi₃ isoform was less abundant and similar to that of Gs protein. At the protein level, these isoforms have also revealed no difference between control and any AIS group (FIG. 10B). These results indicate that the functional changes observed in osteoblasts from AIS patients were unlikely due to aberration in the expression levels of Gi protein isoforms.

### Example 10

### Differential Phosphorylation Patterns Affect Gi Protein Isoforms in AIS Functional Groups

Activity of Gi proteins is acutely regulated by phosphorylation, a process that limits their ability to transduce signals (Casey et al., 1995); (Katada et al., 1985); (Kozasa and Gilman, 1996); (Lounsbury et al., 1991); (Lounsbury et al., 1993); (Morishita et al. 1,995); (Morris et al., 1995); (Yatomi et al., 1992). An increased phosphorylation of serine residues of the three Gi₁, Gi₂ and Gi₃ isoforms of Gi proteins in osteoblasts from AIS patients was previously reported (Moreau et al., 2004). To examine whether the functional disruption in Gi signaling occurring in AIS can be related to the differential patterns of Gi phosphorylated isoforms, Gi isoforms from control subjects and AIS patients from each biological endophenotype were immunoprecipitated and probed with anti-phospho-serine antibody (FIG. 11A-FIG. 11B). Compared with control group, only Gi₂ and Gi₃ isoforms were phosphorylated in the FG3 group, while Gi₁ and Gi₂ only were phosphorylated in the FG2 group. However, the three Gi₁, Gi₂ and Gi₃ isoforms were phosphorylated in FG1 group. These data provide evidence for a relationship between the phosphoprylation pattern of Gi isoforms and the heterogeneous defect of Gi proteins in AIS and are indicative of a difference in the functional status of Gi protein isoforms among AIS groups.

### Example 11

### Differential Phosphorylation Patterns Affect Gi Protein Isoforms in AIS Functional Groups

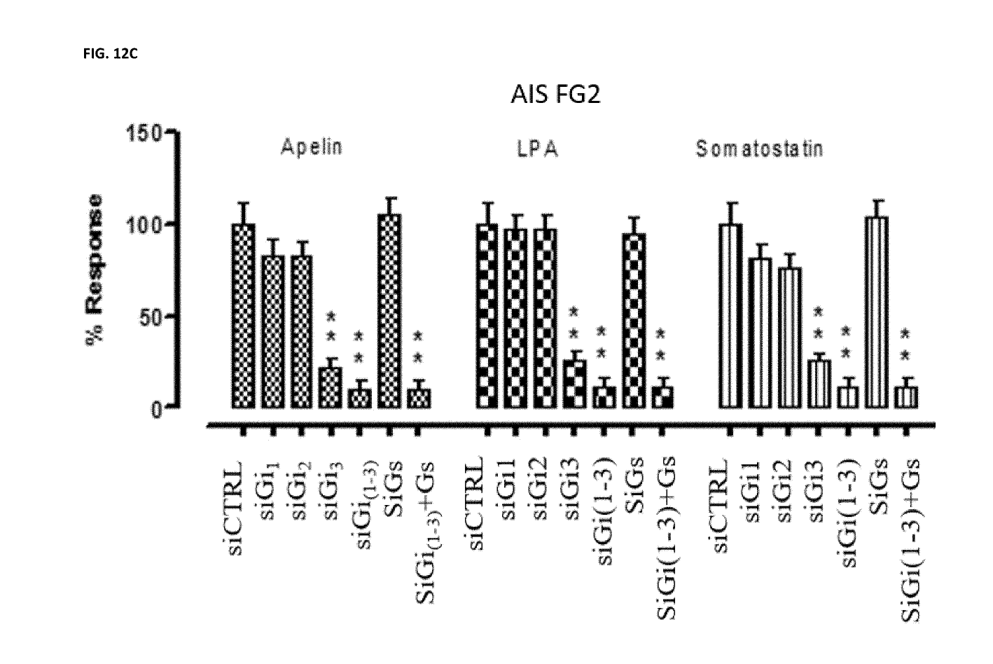

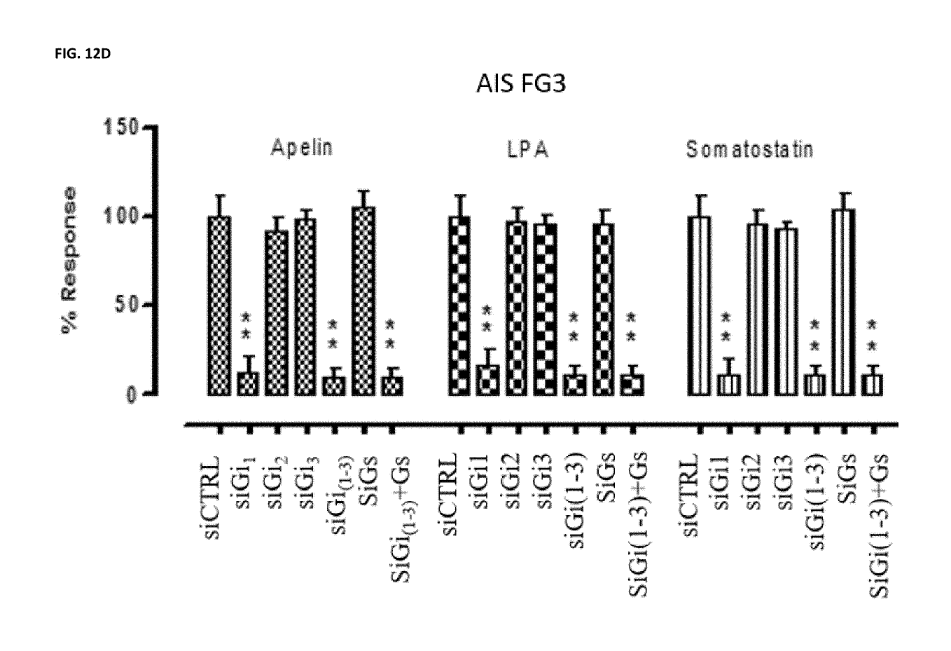

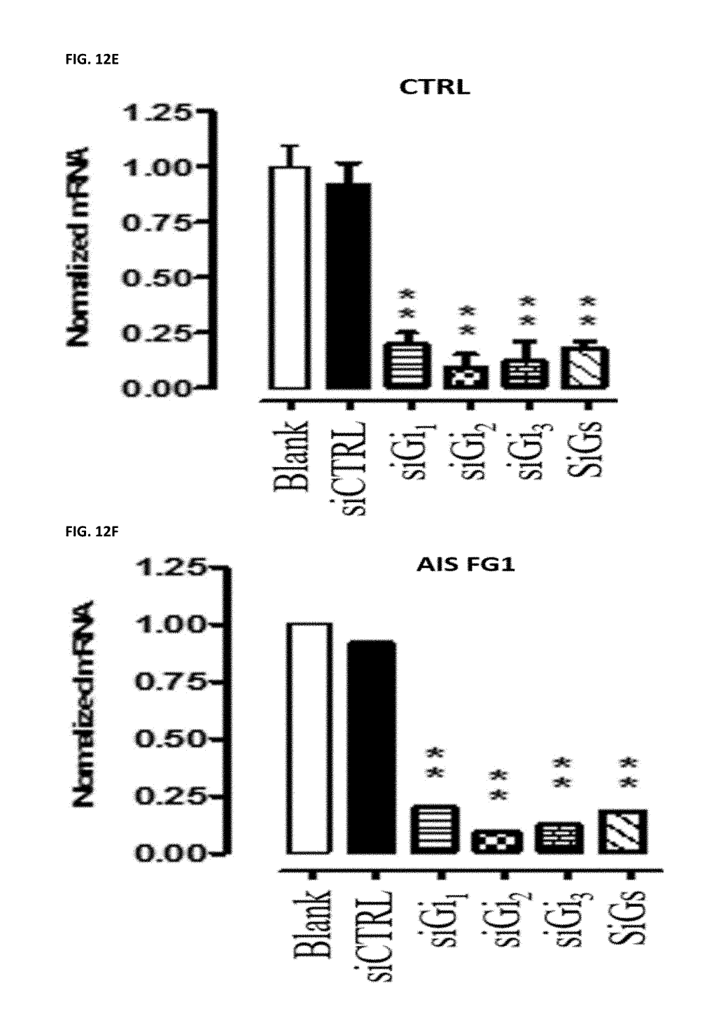

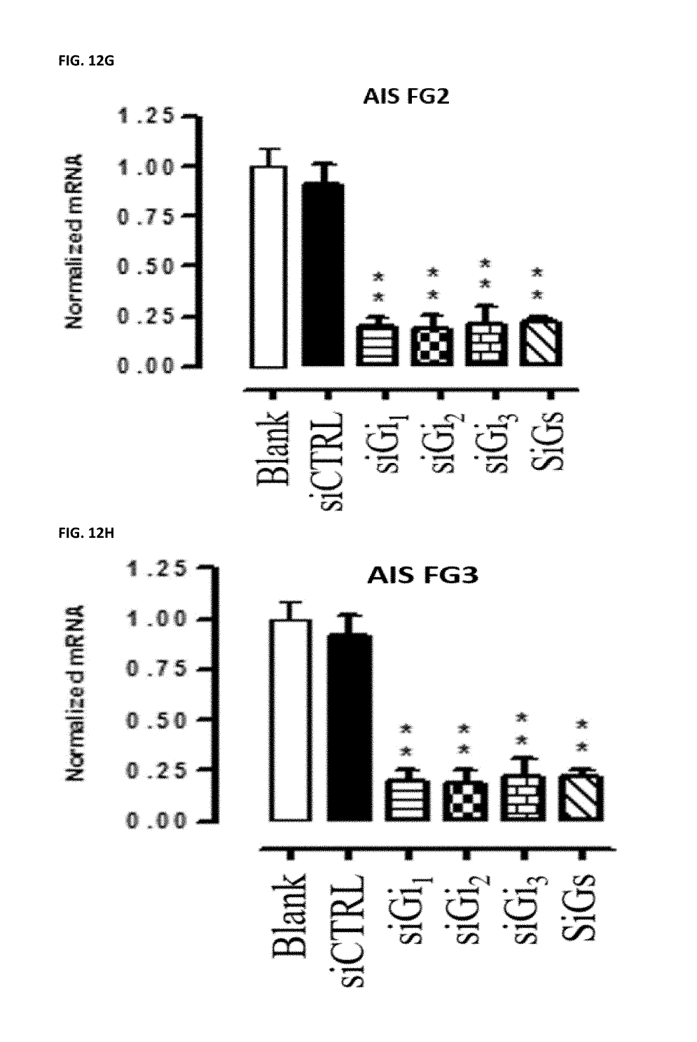

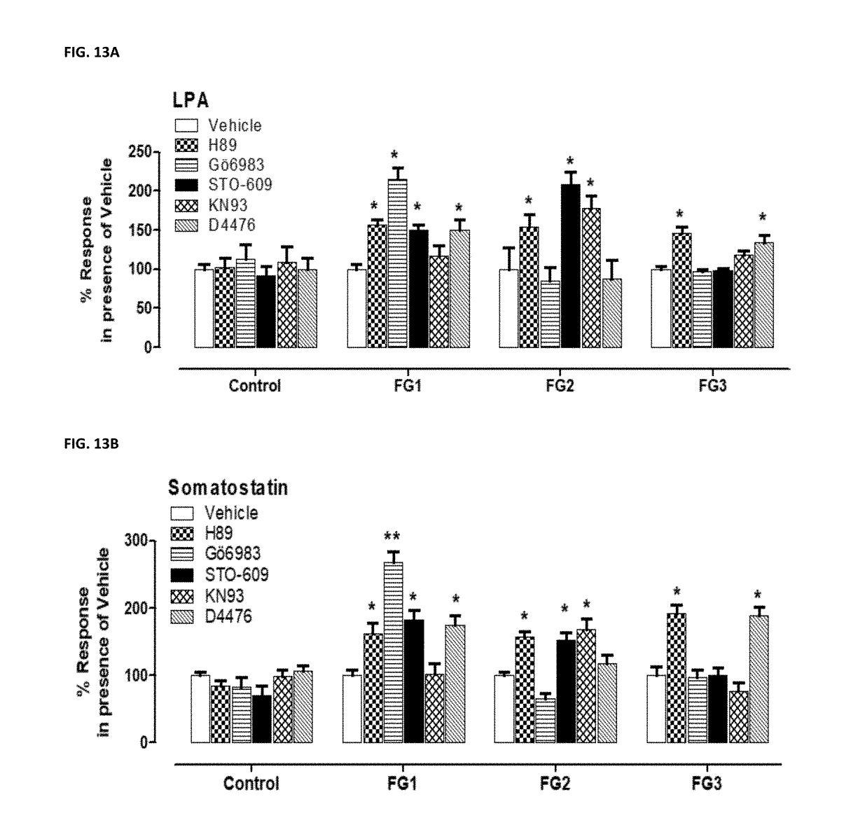

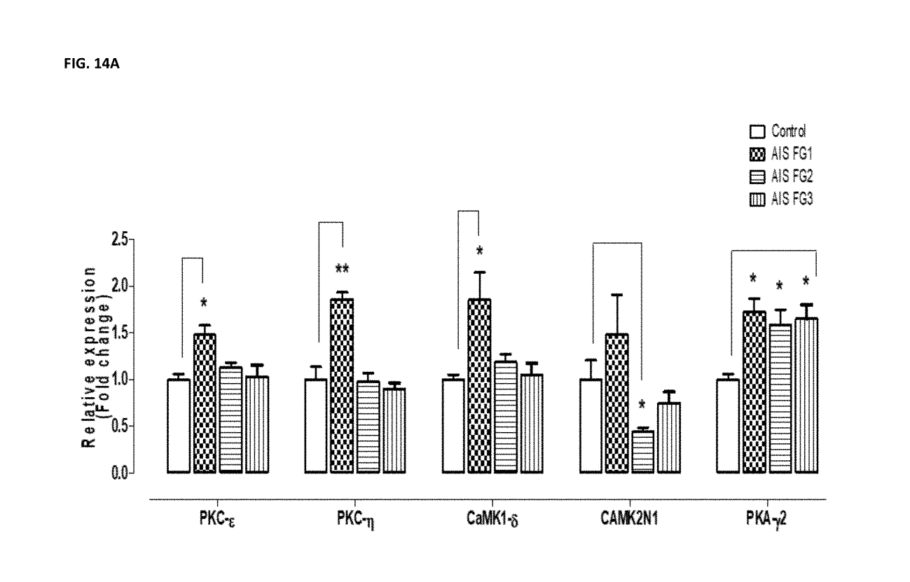

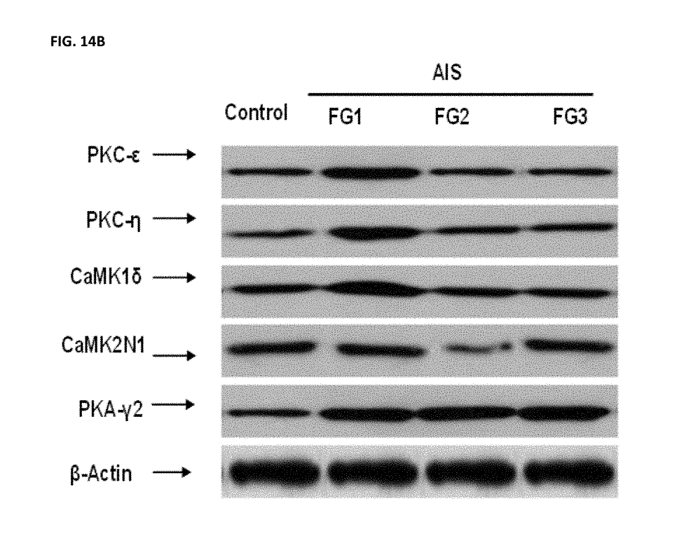

The identity of Gi isoforms responsible for the residual response in each AIS group was tested using a small interference RNA (siRNA) approach to knockdown individually or in combination the expression of Gi₁, Gi₂ Gi₃ and Gs prior to stimulating osteoblasts with melatonin, LPA or somatostatin. Silencing of each gene reduced by 75-85% the expression of the corresponding mRNA in osteoblasts from control and the three AIS groups (FIGS. 12E-H). In the control group, the response to any tested agonist was not significantly affected by Gi₁, Gi₂ Gi₃ siRNA when transfected alone, but was almost abolished when transfected together (FIG. 12A). The response to each tested agonist was reduced by at least 75% by silencing Gi₃ alone in FG2 osteoblasts (FIG. 12C), and by 90% by silencing Gi₁ alone in FG3 osteoblasts (FIG. 12D) confirming that the residual response to Gi stimulation is mediated by Gi₃ and Gi₁ isoforms in FG2 and FG3 groups, respectively. In FG1 osteoblasts, response to each agonist was reduced by 50% following the depletion of all Gi isoforms by siRNA, and was not affected by the knockdown of individual Gi isoforms (FIG. 12B). In contrast, the depletion of Gs alone reduced the cellular response to any tested agonist by 50% in the FG1 AIS group, and was devoid of effect in the control, FG2 and FG3 AIS groups. This indicates that the residual response to Gi stimulation in the FG1 functional group is an additive effect of Gi-receptors coupling simultaneously to Gs and Gi proteins.

The detection of unphosphorylated Gi₃ and Gi₁ isoforms respectively in FG2 and FG3 groups could explain their higher Gi signaling activity when compared to FG1 group. Nevertheless, FG2 osteoblasts exhibit a much weaker residual Gi signaling activity when compared to FG3 osteoblasts, which could be explained by the fact that Gi₃ isoform is less abundant in human osteoblasts as demonstrated in Example 9 (FIG. 10A-FIG. 10B). The selective depletion of Gi₃ and Gi₁ isoforms in FG2 and FG3 osteoblasts, respectively almost abolished their cellular responses to three distinct Gi-coupled receptors in a similar manner as demonstrated in FIG. 12A-FIG. 12H, further confirming the functional status of Gi₃ and Gi₁ isoforms in these two AIS groups. It is tempting to speculate that the loss of function of other Gi isoforms may be compensated by Gi₃ isoform in FG2 and by Gi₁ in FG3 group, which is consistent with the concept that each Gi isoform can partially rescue Gi signaling (Hurst et al., 2008).

Without being bound by this hypothesis, these findings, together with results from the experiments with high PTX concentrations, suggest the presence of a compensatory mechanism involving other G proteins in FG1 osteoblasts. Conceptually, it is possible that phosphorylation of all Gi isoforms allows or facilitates the coupling of Gs proteins to Gi-coupled receptors in FG1 osteoblasts. Such possibility was clearly demonstrated by the simultaneous deletion of Gs and all Gi isoforms, which almost abrogated completely the signaling activity in FG1 osteoblasts. On the other hand, the three GPCRs tested did not activate Gs mediated signaling in FG2 and FG3 osteoblasts, which strongly suggests that Gs protein access to these receptors is limited by the presence of Gi₃ and Gi₁ isoforms, respectively, which are not phosphorylated in these groups. Of note, depletion of Gs proteins alone did not abolish the signaling activity in FG1 osteoblasts despite the fact that all three Gi isoforms were phosphorylated suggesting a possible heterogeneity in the number and position of serine residues phosphorylated among AIS functional groups. It is conceivable that phosphorylated Gi proteins in FG1 osteoblasts still exhibit some residual activity as opposed to phosphorylated Gi isoforms in FG2 and FG3 groups.

### Example 12

### Identification of Serine/Threonine Kinases Contributing to Gi Proteins Hypofunctionality in AIS

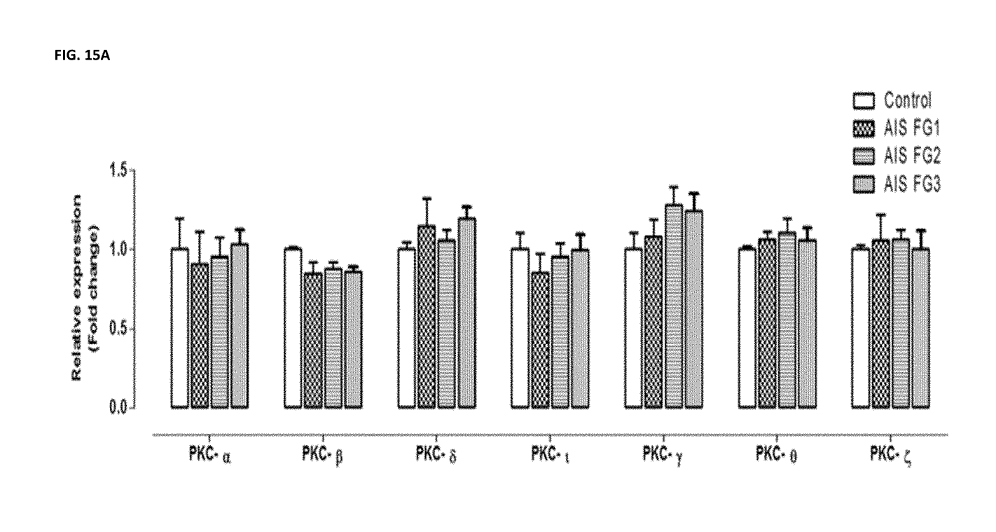

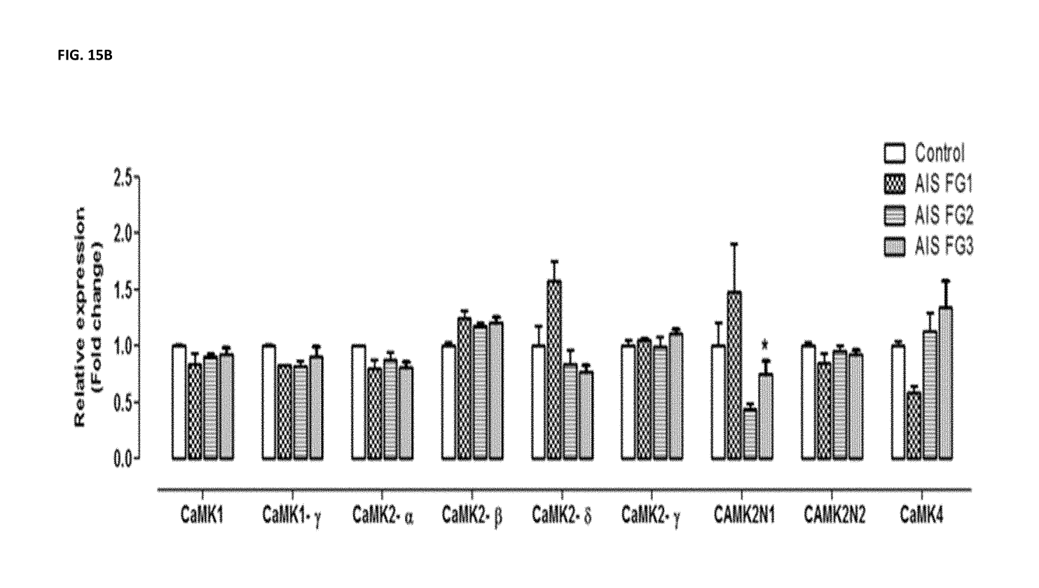

In order to identify putative serine/threonine kinases phosphorylating Gi isoforms in AIS, control and AIS osteoblasts were treated with a panel of serine/threonine kinase inhibitors prior to their stimulation with two distinct agonists (LPA and somatostatin). Of note, Gi stimulation induced via LPA or somatostatin receptor activation by their agonists in control osteoblasts was not affected by any kinase inhibitor (FIG. 13A-FIG. 13B). In contrast, signaling responses was significantly increased in FG1 osteoblasts by Gö8963, which inhibits several isoforms of PKC and also by STO-609 acetate, which inhibits three isoforms of calmodulin kinase (CaMK1, CaMK2, CaMK4), but was unaffected by KN93 that inhibits selectively CaMK2. However, both CaMK inhibitors STO-609 acetate and KN93 increased the signalling response in osteoblasts from FG2 group, while Gö8963 was devoid of significant effect in this AIS group. Cellular response in FG3 osteoblasts was not improved by inhibition of neither of PKC nor CaMK. In contrast, the inhibition of Casein Kinase 2 (CK2) with D4476 increased response in osteoblasts from FG1 and FG3 groups, but not in FG2 osteoblasts, while the inhibition of PKA with H89 increased the signalling response in all AIS groups.

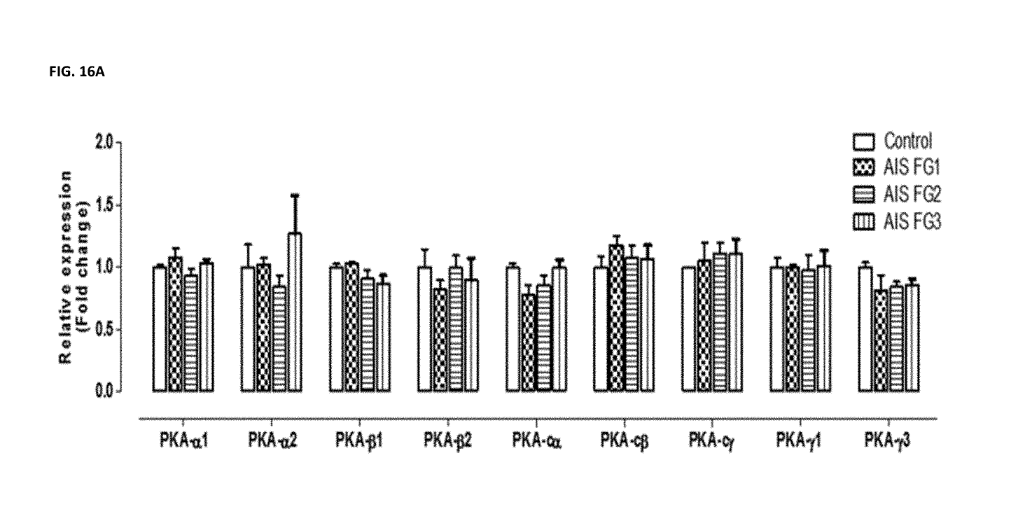

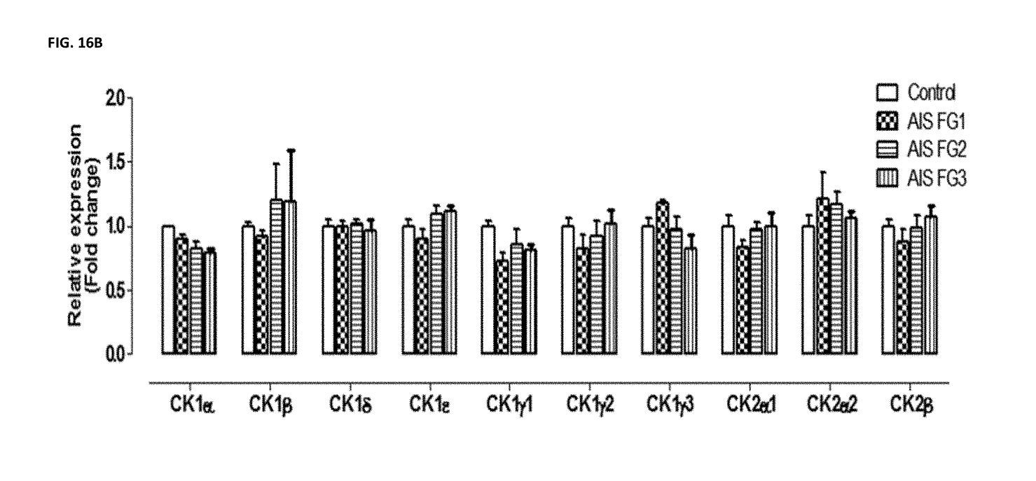

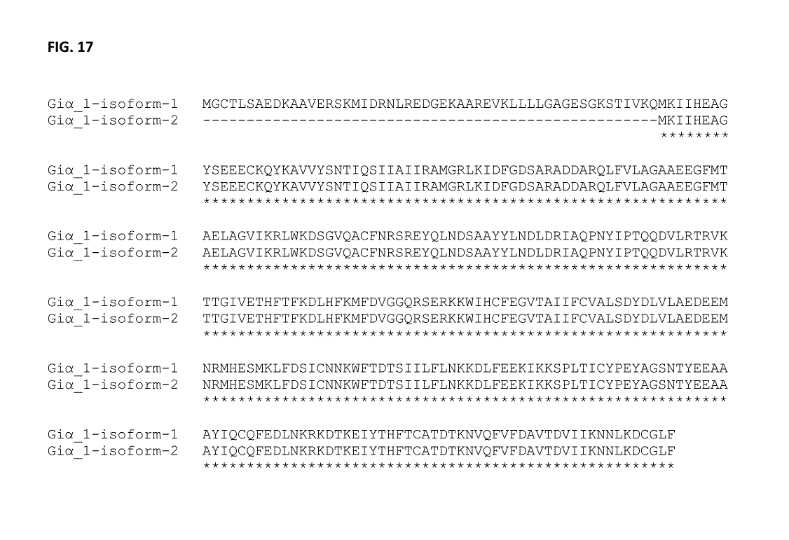

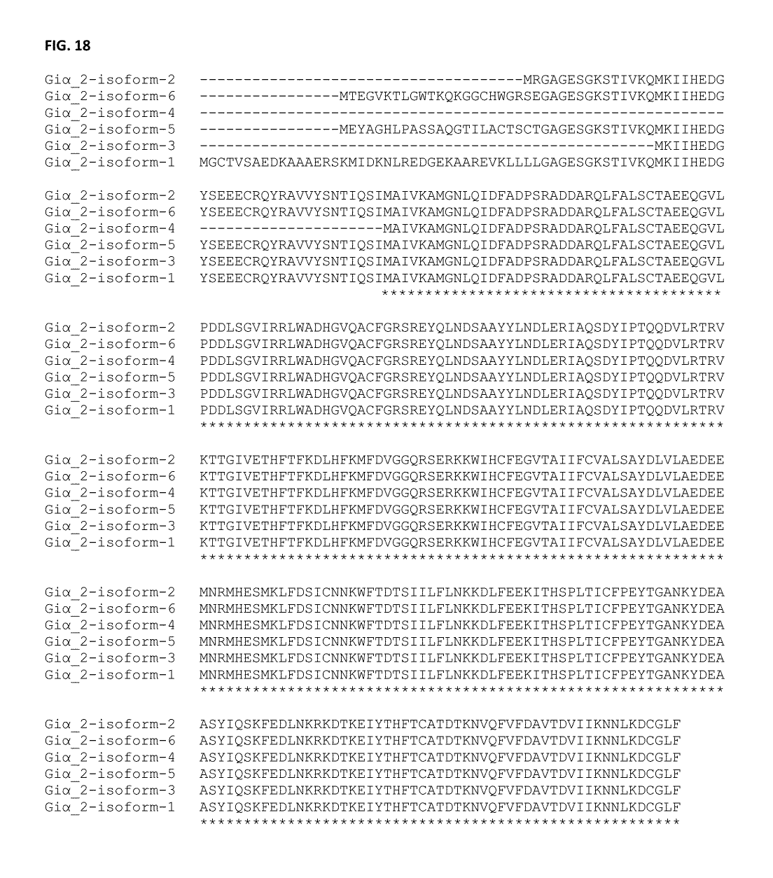

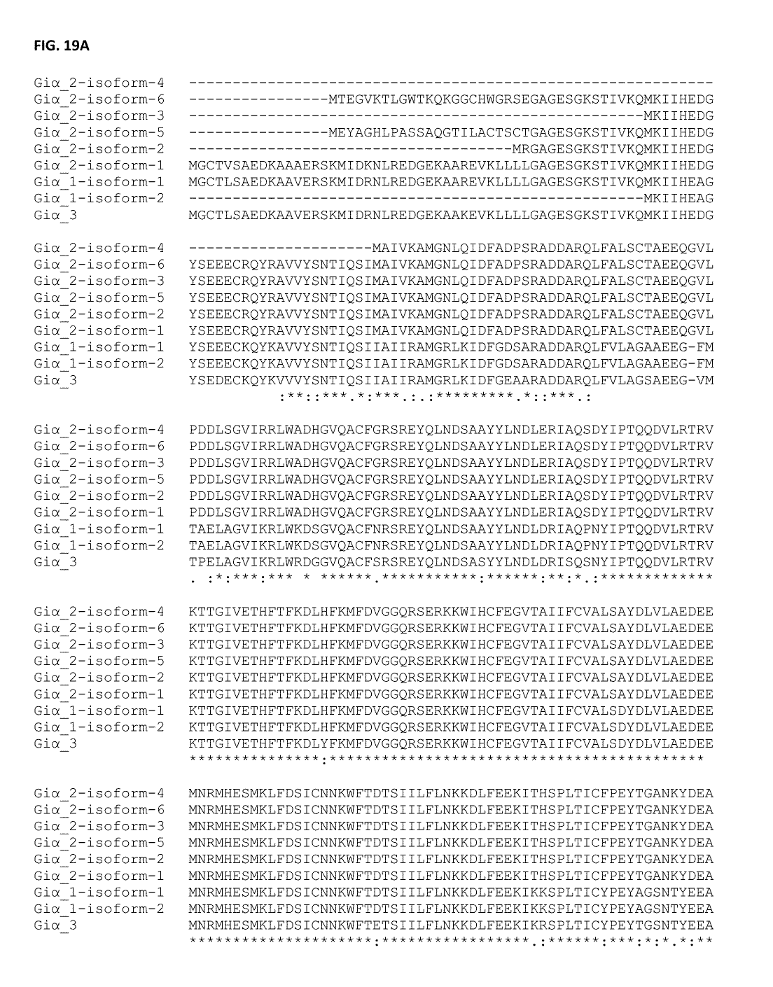

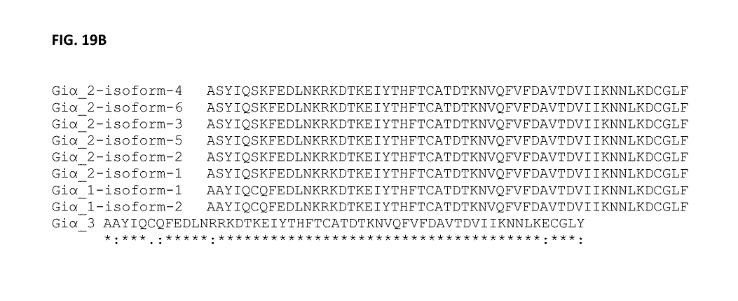

Expression analysis has revealed a selective increase in the expression levels of PKCε, PKCη and CaMK1-δ in FG1 osteoblasts when compared to control group (FIG. 14A-FIG. 14B). FG2 and FG3 osteoblasts have exhibited high expression levels of PKAγ2 only. In contrast, the expression level of CaMK2n1, a natural CaMK2 inhibitor, was significantly decreased in FG2 osteoblasts, suggesting that the regulatory system of CaMK2 activity is affected in this AIS group (FIG. 14A-FIG. 14B). The expression levels of other analysed kinases did not show any significant selective increase or decrease (FIG. 151-FIG. 15B and FIG. 16A-FIG. 16B) Collectively, these results show that selective functional alteration of Gi protein isoforms in AIS involves distinct kinases.

The scope of the claims should not be limited by the preferred embodiments set forth in the examples, but should be given the broadest interpretation consistent with the description as a whole.

### REFERENCES

1. Akoume, M.-Y., B. Azeddine, et al. (2010). “Cell-based screening test for idiopathic scoliosis using cellular dielectric spectroscopy.” Spine 35(13): E601.
2. Anand-Srivastava, M. B. (1989). “Amiloride interacts with guanine nucleotide regulatory proteins and attenuates the hormonal inhibition of adenylate cyclase.” J Biol Chem 264(16): 9491-9496.
3. Anand-Srivastava, M. B., A. K. Srivastava, et al. (1987). “Pertussis toxin attenuates atrial natriuretic factor-mediated inhibition of adenylate cyclase. Involvement of inhibitory guanine nucleotide regulatory protein.” J Biol Chem 262(11): 4931-4934.
4. Azeddine, B., K. Letellier, et al. (2007). “Molecular determinants of melatonin signaling dysfunction in adolescent idiopathic scoliosis.” Clin Orthop Relat Res 462: 45-52.
5. Blank, R. D., C. L. Raggio, et al. (1999). “A genomic approach to scoliosis pathogenesis.” Lupus 8(5): 356-360.
6. Bushfield, M., S. L. Griffiths, et al. (1990). “Diabetes-induced alterations in the expression, functioning and phosphorylation state of the inhibitory guanine nucleotide regulatory protein Gi-2 in hepatocytes.” Biochem J 271(2): 365-372.
7. Bushfield, M., B. E. Lavan, et al. (1991). “Okadaic acid identifies a phosphorylation/dephosphorylation cycle controlling the inhibitory guanine-nucleotide-binding regulatory protein Gi2.” Biochem J 274 (Pt 2): 317-321.
8. Casey, J. H., C. J. McLean, et al. (1995). “Driving, glaucoma, and the law. Diabetic patients must also satisfy regulations.” BMJ 310(6971): 56.
9. Chen, C. A. and D. R. Manning (2001). “Regulation of G proteins by covalent modification.” Oncogene 20(13): 1643-1652.
10. Fazal, M. A. and M. Edgar (2006). “Detection of adolescent idiopathic scoliosis.” Acta orthopaedica belgica 72(2): 184.
11. Gawler, D., G. Milligan, et al. (1987). “Abolition of the expression of inhibitory guanine nucleotide regulatory protein Gi activity in diabetes.” Nature 327(6119): 229-232.
12. Hardeland, R. (2009). “Melatonin: signaling mechanisms of a pleiotropic agent.” Biofactors 35(2): 183-192.
13. Higashijima, T., J. Burnier, et al. (1990). “Regulation of Gi and Go by mastoparan, related amphiphilic peptides, and hydrophobic amines. Mechanism and structural determinants of activity.” J Biol Chem 265(24): 14176-14186.
14. Houslay, M. D. and G. Milligan (1997). “Tailoring cAMP-signalling responses through isoform multiplicity.” Trends Biochem Sci 22(6): 217-224.
15. Itoh, H., F. Okajima, et al. (1984). “Conversion of adrenergic mechanism from an alpha- to a beta-type during primary culture of rat hepatocytes. Accompanying decreases in the function of the inhibitory guanine nucleotide regulatory component of adenylate cyclase identified as the substrate of islet-activating protein.” J Biol Chem 259(24): 15464-15473.
16. Kane, W. J. (1977). “Scoliosis prevalence: a call for a statement of terms.” Clin Orthop Relat Res (126): 43-46.
17. Katada, T., A. G. Gilman, et al. (1985). “Protein kinase C phosphorylates the inhibitory guanine-nucleotide-binding regulatory component and apparently suppresses its function in hormonal inhibition of adenylate cyclase.” Eur J Biochem 151(2): 431-437.
18. Kozasa, T. and A. G. Gilman (1996). “Protein kinase C phosphorylates G12 alpha and inhibits its interaction with G beta gamma.” J Biol Chem 271(21): 12562-12567.
19. Letellier, K., B. Azeddine, et al. (2008). “Estrogen cross-talk with the melatonin signaling pathway in human osteoblasts derived from adolescent idiopathic scoliosis patients.” Journal of pineal research 45(4): 383-393.
20. Lonstein, J. (1994). “Adolescent idiopathic scoliosis.” The lancet 344(8934): 1407-1412.
21. Lounsbury, K. M., P. J. Casey, et al. (1991). “Phosphorylation of Gz in human platelets. Selectivity and site of modification.” J Biol Chem 266(32): 22051-22056.
22. Lounsbury, K. M., B. Schlegel, et al. (1993). “Analysis of Gz alpha by site-directed mutagenesis. Sites and specificity of protein kinase C-dependent phosphorylation.” J Biol Chem 268(5): 3494-3498.
23. McClue, S., E. Seizer, et al. (1992). “Gi3 does not contribute to the inhibition of adenylate cyclase when stimulation of an alpha 2-adrenergic receptor causes activation of both Gi2 and Gi3.” Biochemical Journal 284(Pt 2): 565.
24. Moreau, A., M. Y. Akoume Ndong, et al. (2009). “Molecular and genetic aspects of idiopathic scoliosis. Blood test for idiopathic scoliosis.” Orthopade 38(2): 114-116, 118-121.
25. Moreau, A., S. Forget, et al. (2004). “Melatonin signaling dysfunction in adolescent idiopathic scoliosis.” Spine 29(16): 1772-1781.
26. Morishita, R., H. Nakayama, et al. (1995). “Primary structure of a gamma subunit of G protein, gamma 12, and its phosphorylation by protein kinase C.” J Biol Chem 270(49): 29469-29475.
27. Morris, N. J., P. Young, et al. (1995). “Insulin inhibits the phosphorylation of alpha-Gi-2 in intact hepatocytes.” Biochem J 308 (Pt 2): 693-696.
28. Mukai, H., E. Munekata, et al. (1992). “G protein antagonists. A novel hydrophobic peptide competes with receptor for G protein binding.” J Biol Chem 267(23): 16237-16243.
29. Murthy, K. S., J. R. Grider, et al. (2000). “Heterologous desensitization of response mediated by selective PKC-dependent phosphorylation of G(i-1) and G(i-2).” Am J Physiol Cell Physiol 279(4): C925-934.
30. Negrini, S., S. Minozzi, et al. (2010). “Cochrane Review: Braces for idiopathic scoliosis in adolescents.” Evidence-Based Child Health: A Cochrane Review Journal 5(4): 1681-1720.
31. Nonno, R., M. Pannacci, et al. (1999). “Ligand efficacy and potency at recombinant human MT2 melatonin receptors: evidence for agonist activity of some mt1-antagonists.” Br J Pharmacol 127(5): 1288-1294.
32. Riseborough, E. J. and R. Wynne-Davies (1973). “A genetic survey of idiopathic scoliosis in Boston, Mass.” J Bone Joint Surg Am 55(5): 974-982.
33. Roach, J. W. (1999). “Adolescent idiopathic scoliosis.” Orthop Clin North Am 30(3): 353-365, vii-viii.
34. Verdonk, E., K. Johnson, et al. (2006). “Cellular dielectric spectroscopy: a label-free comprehensive platform for functional evaluation of endogenous receptors.” Assay and drug development technologies 4(5): 609-619.
35. Weinstein, S. L. (1989). “Adolescent idiopathic scoliosis: prevalence and natural history.” Instr Course Lect 38: 115-128.
36. Wesslau, C. and U. Smith (1992). “The inhibitory GTP-binding protein (Gi) regulates the agonistic property of beta-adreneraic ligands in isolated rat adipocytes. Evidence for a priming effect of cyclic AMP.” Biochem J 288 (Pt 1): 41-46.
37. Yatomi, Y., Y. Arata, et al. (1992). “Phosphorylation of the inhibitory guanine-nucleotide-binding protein as a possible mechanism of inhibition by protein kinase C of agonist-induced Ca2+ mobilization in human platelet.” Eur J Biochem 205(3): 1003-1009.

1. A kit for stratifying a subject having adolescent idiopathic scoliosis (AIS), comprising: (i) a cell sample from the subject, (ii) pertussis toxin (PTX), (iii) a ligand for stimulating the activity of Giα proteins in the cell sample from the subject, and (iv) a ligand for stimulating the activity of Gsα proteins in the cell sample from the subject, wherein at least one of said ligand in (iii) or (iv) is a non-naturally occurring ligand.

2. The kit of claim 1, wherein said cell sample comprises peripheral blood mononuclear cells (PBMC).

3. The kit of claim 1, wherein said cell sample comprises myoblasts.

4. The kit of claim 1, wherein said cell sample comprises osteoblasts.

5. The kit of claim 1, wherein said ligand for stimulating the activity of Giα proteins is melatonin, Apelin, PB554, LPA, somatostatin, or UK14304.

6. The kit of claim 1, wherein said ligand for stimulating the activity of Gsα proteins is isoproterenol or desmopressine.
